(19) United States
(12) Patent Application Publication
(10) Pub. No.: US 2025/0215002 A1
(43) Pub. Date: Jul. 3, 2025

(54) SOLID FORMS OF TYK2 INHIBITORS AND METHODS OF USE

(71) Applicant: Takeda Pharmaceutical Company Limited, Osaka (JP)

(72) Inventors: Alan John COLLIS, Lexington, MA (US); Jon P. LAWSON, Boston, MA (US); Melanie Janelle BEVILL, West Lafayette, IN (US); Stephan PARENT, West Lafayette, IN (US)

(21) Appl. No.: 18/850,485

(22) PCT Filed: Mar. 24, 2023
(86) PCT No.: PCT/US2023/064916

§ 371 Date: Sep. 24, 2024

Related U.S. Application Data
(60) Provisional application No. 63/269,944, filed on Mar. 25, 2022.

(51) Int. Cl. C07D 487/04

(52) U.S. Cl. CPC C07D 487/04

(57) ABSTRACT

Solid forms of TYK2 inhibitors, pharmaceutical compositions thereof and methods of treatment are described herein.

### PRIORITY CLAIM

This application claims priority from U.S. Provisional Patent Application No. 63/269,944, filed Mar. 25, 2022, which is incorporated by reference in its entirety.

### TECHNICAL FIELD

The present invention relates to solid forms of a tyrosine-protein kinase 2 (TYK2) inhibitor and to methods of making such solid forms. The invention also provides methods of treating disorders using pharmaceutical compositions comprising such solid forms.

### BACKGROUND

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxins, and H₂O₂), cytokines (e.g., interleukin-1 (IL-1), interleukin-8 (IL-8), and tumor necrosis factor a (TNF-a)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

### SUMMARY

In one aspect, s solid form of Compound 1:

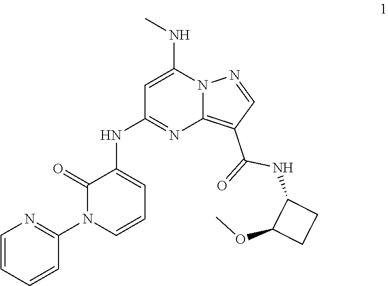 [SMILES: CNc1cc(Nc2cccn(-c3ccccn3)c2=O)nc2c(C(=O)N[C@@H]3CC[C@H]3OC)cnn12]

### can be of Form C.

In another aspect, a solid form of Compound 1:

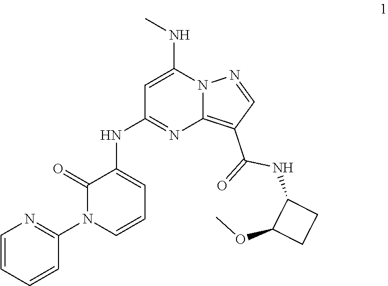 [SMILES: CNc1cc(Nc2cccn(-c3ccccn3)c2=O)nc2c(C(=O)N[C@@H]3CC[C@H]3OC)cnn12]

### can be of Form A.

In some aspects, a crystalline Form C of Compound 1 is provided:

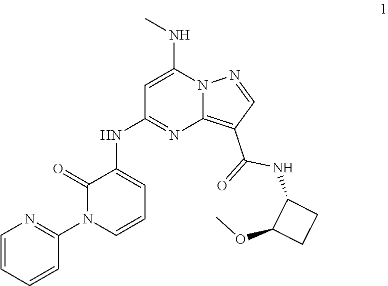 [SMILES: CNc1cc(Nc2cccn(-c3ccccn3)c2=O)nc2c(C(=O)N[C@@H]3CC[C@H]3OC)cnn12]

comprising an XRPD pattern substantially as shown in FIG. 10 (top trace).

In another aspect, A solid form of Compound 1:

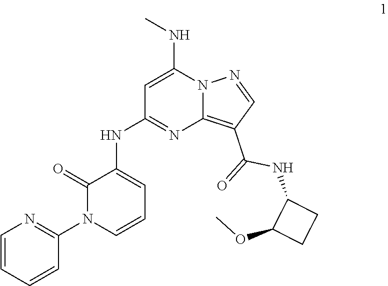 [SMILES: CNc1cc(Nc2cccn(-c3ccccn3)c2=O)nc2c(C(=O)N[C@@H]3CC[C@H]3OC)cnn12]

### can be of Form A, Form B, Form C, Form D, Form E, Form F, Form G, Form H, Form I, or Form J.

In some aspects, a crystalline Form A of Compound 1 is provided:

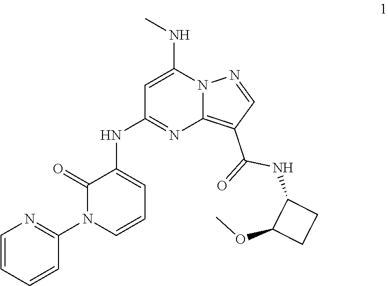 [SMILES: CNc1cc(Nc2cccn(-c3ccccn3)c2=O)nc2c(C(=O)N[C@@H]3CC[C@H]3OC)cnn12]

comprising an XRPD pattern substantially as shown in FIG. 5. In some embodiments, Form A is a hydrate. In some embodiments, Form A is a monohydrate.

In another aspect, methods of treating disorders can include using the crystalline polymorphs of Compound 1.

In another aspect, a pharmaceutical composition can include a solid form as described herein and a pharmaceutically acceptable carrier, excipient, or adjuvant

In certain embodiments, the solid form can be substantially free of impurities.

In certain embodiments, the solid form can be a crystalline solid substantially free of amorphous Compound 1.

In certain embodiments, the solid form can be characterized by having at least 3, 4, 5, or 6 peaks of an X-ray powder diffraction pattern (XRPD) of FIG. 16 (bottom trace).

In certain embodiments, the solid form can be characterized by having at least 3, 4, 5, or 6 peaks of an X-ray powder diffraction pattern (XRPD) of FIG. 10.

In certain embodiments, the solid form exhibits an X-ray powder diffraction pattern (XRPD) that is substantially similar to that of FIG. 10 (top trace) or FIG. 16 (bottom trace).

In certain embodiments, the solid form exhibits characterized by a TGA analysis of mass loss between 165° C. and 175° C.

In certain embodiments, the solid form exhibits characterized by a DSC comprising a peak onset at about 247° C.

In certain embodiments, the solid form exhibits characterized by a DSC substantially as shown in FIG. 12.

In certain embodiments, the solid form can be characterized by a DVS isotherm substantially as shown in FIG. 13.

In certain embodiments, the solid form can be a hydrate.

In certain embodiments, the solid form can be a monohydrate.

In certain embodiments, the solid form can be a dihydrate.

In certain embodiments, the solid form can be a trihydrate.

In certain embodiments, the solid form can be characterized by having at least 3, 4, 5, or 6 peaks of an X-ray powder diffraction pattern (XRPD) of FIG. 4 (bottom trace) or FIG. 5.

In certain embodiments, the solid form can be characterized by having an X-ray powder diffraction pattern (XRPD) that is substantially similar to that of FIG. 4 (bottom trace) or FIG. 5.

In certain embodiments, the solid form can be characterized by having at least 3, 4 or 5 of the highest amplitude peaks of the XRPD of FIG. 5.

In certain embodiments, the solid form can be characterized by a TGA analysis of mass loss between 65° C. and 130° C.

In certain embodiments, the solid form can have a TGA substantially as shown in FIG. 7.

In certain embodiments, the solid form can be characterized by a DSC comprising peak onsets at about 91° C., 103° C., and 245° C. For example, the peak onset can consist essentially of 245° C.

In certain embodiments, the solid form can have a DSC substantially as shown in FIG. 7.

In certain embodiments, the solid form can exhibit peaks or other physical properties as shown in any one of FIG. 1 to FIG. 42.

Other aspects, embodiments, and features will be apparent from the following description, the drawings, and the claims.

### BRIEF DESCRIPTION OF THE FIGURES

FIG. 1 shows the numbering of the non-hydrogen atoms of Compound 1.

FIG. 2 depicts the XRPD pattern of Compound 1 Form A-E overlay.

FIG. 3 depicts the XRPD overlay of Compound 1 F (top) through J (bottom).

FIG. 4 depicts an XRPD overlay Form A of Compound 1.

FIG. 5 depicts the XRPD for Form A of Compound 1.

FIG. 6 depicts the DSC and TGA thermograms for Compound 1 Form A+minor Material B of Compound 1.

FIG. 7 shows a DSC and TGA thermograms for Compound 1, Form A.

FIG. 8 depicts DVS isotherm for Compound 1 Form A+minor Material B.

FIG. 9 shows an atomic displacement ellipsoid diagram of Compound 1, Form C.

FIG. 10 shows experimental and calculated XRPD patterns of Compound 1 Form C.

FIG. 11 shows tentative XRPD indexing solution for Compound 1, Form C.

FIG. 12 shows DSC and TGA thermograms for Compound 1 Form C.

FIG. 13 shows the DVS isotherm for Compound 1 Form C.

FIG. 14 shows illustration of void spaces in crystal structure for Compound 1 Form C.

FIG. 15 shows cycling DSC thermogram for Form C of Compound 1.

FIG. 16 shows XRPD overlay of Material D and Form C patterns of Compound 1.

FIG. 17 shows tentative XRPD indexing solution for Compound 1 Material D.

FIG. 18 shows XRPD overlay of Material E patterns of Compound 1.

FIG. 19 shows XRPD overlay of Material G and Form A patterns of Compound 1.

FIG. 20 shows DSC and TGA thermograms for Compound 1 Material G+minor Form A.

FIG. 21 shows the DSC and TGA thermograms for Compound 1 Material H.

FIG. 22 shows XRPD overlay of Form A, Form C, and Material I mixture of Compound 1.

FIG. 23 shows XRPD overlay of Form J patterns of Compound 1.

FIG. 24 shows tentative XRPD indexing solution for Compound 1 Form J.

FIG. 25 shows DSC thermogram for Compound 1 Form J.

FIG. 26 shows DSC and TGA thermograms for Compound 1 Form J.

FIG. 27 shows a TGA thermogram for heating experiment for Compound 1 Form A.

FIG. 28 shows cycling TGA thermogram for heating experiment for Form C of Compound 1.

FIG. 29 shows TGA thermogram for heating experiment for Compound 1 Material H.

FIG. 30 shows cycling TGA/DSC thermograms for heating experiment for Compound 1 Form C.

FIG. 31 shows form A minor material B of Compound 1.

FIG. 32 shows post DVS Form A minor material B of Compound 1.

FIG. 33 shows Form A XRPD of Compound 1.

FIG. 34 shows additional Form A XRPD of Compound 1.

FIG. 35 shows Form C XRPD of Compound 1.

FIG. 36 shows additional run of From C XRPD of Compound 1.

FIG. 37 shows post DVS XRPD of Compound 1.

FIG. 38 shows Material D anhydrous nonsolvated of Compound 1.

FIG. 39 shows Material E disordered of Compound 1.

FIG. 40 shows XRPD of Material F disordered of Compound 1.

FIG. 41 shows Form G+minor A.

FIG. 42 shows XRPD of Form A of Compound 1.

### DETAILED DESCRIPTION

TYK2 catalyzes the phosphorylation of STAT proteins downstream of a number of cytokine receptors, including the Type I interferon receptor and the IL-12 and IL-23 receptors. The activation of TYK2-dependent receptors by their cytokine ligands results in the activation of STAT-dependent transcription and cellular functional responses specific for the receptors and cell types on which they are expressed. The cytokine signaling pathways regulated by TYK2 play key roles in several immune-mediated disorders. The cytokine IL-12 is essential for the development of Type 1 T-helper cells (Th1) which produce interferon-gamma, a major effector molecule in systemic autoimmune disorders such as systemic lupus erythematosus. The cytokine IL-23 is central for the expansion and survival of Th17 cells and innate lymphoid cells, both of which have been shown to play key pathogenic roles in autoimmunity. IL-23 stimulation drives the production of key proinflammatory cytokines by Th17 cells, including IL-17A, IL-17F, and IL-22, all of which are effector molecules important for pathogenesis of conditions such as psoriasis, psoriatic arthritis, and spondylarthritis. Inhibition of TYK2 would be expected to impact multiple immune-mediated disorders through its effects on the IL-23/Th17/Th22 axis, IL-12-mediated Th1 functions, and Type I interferon-driven modulation of diverse immune pathways and cell types.

### Compound 1:

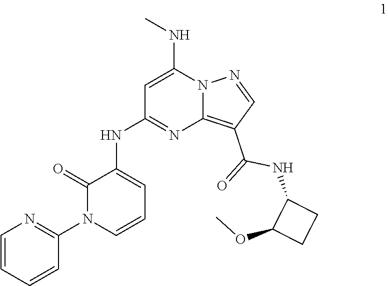 [SMILES: CNc1cc(Nc2cccn(-c3ccccn3)c2=O)nc2c(C(=O)N[C@@H]3CC[C@H]3OC)cnn12]

is a TYK2 inhibitor previously disclosed by Applicant. See U.S. Pat. No. 11,046,698. Various crystalline polymorphs of a compound (e.g., Compound 1) can vary the dissolution, stability, hygroscopicity and bioavailability of the compound. The present disclosure satisfies the need for elucidating stable polymorphic forms of Compound 1 and provides other related advantages.

It would be desirable to provide a solid form of Compound 1 (e.g., as a freebase thereof or salt thereof) that imparts characteristics such as improved aqueous solubility, stability and ease of formulation. As described herein Compound 1 described herein or a compound described herein can be a compound in a solid form identified herein. Accordingly, the solid forms described herein can include either free base forms and salt forms of Compound 1:

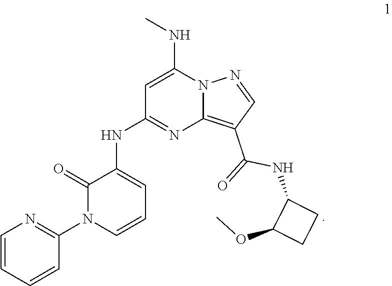 [SMILES: CNc1cc(Nc2cccn(-c3ccccn3)c2=O)nc2c(C(=O)N[C@@H]3CC[C@H]3OC)cnn12]

### Free Base Forms of Compound 1

In one aspect, Form C of Compound 1 as a crystalline solid:

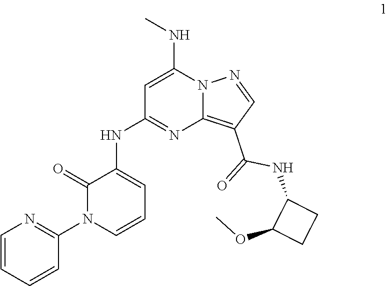 [SMILES: CNc1cc(Nc2cccn(-c3ccccn3)c2=O)nc2c(C(=O)N[C@@H]3CC[C@H]3OC)cnn12]

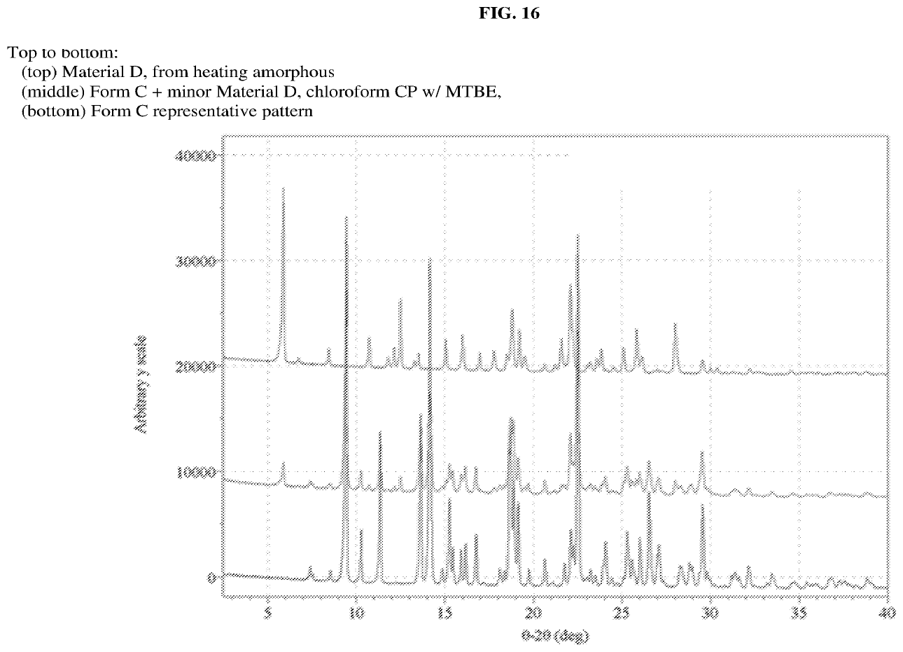

can include an X-ray powder diffraction pattern (XRPD) that is substantially similar to that of FIG. 16 (bottom trace).

In one aspect, a solid form of Compound 1:

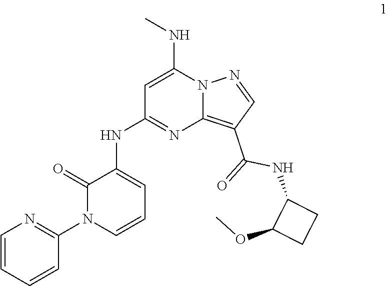 [SMILES: CNc1cc(Nc2cccn(-c3ccccn3)c2=O)nc2c(C(=O)N[C@@H]3CC[C@H]3OC)cnn12]

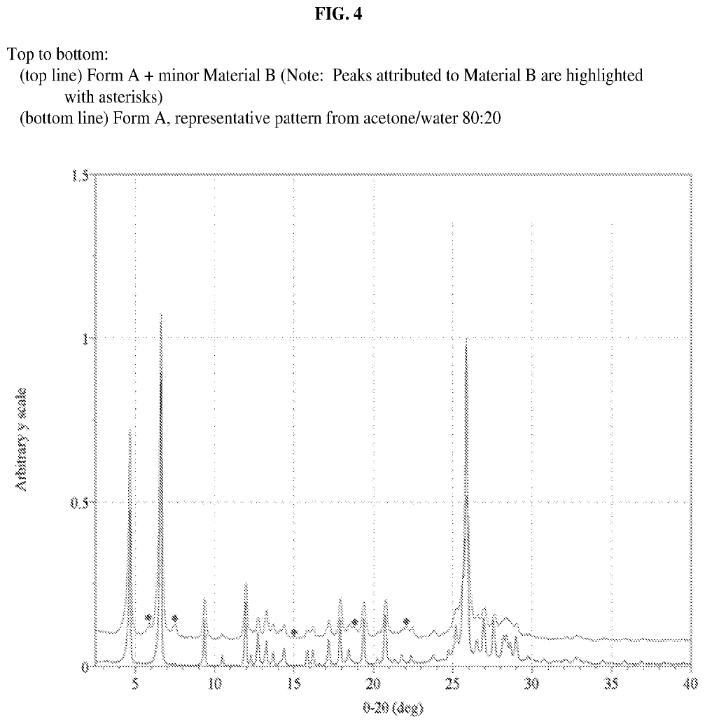

can be of Form A. In some embodiments, the solid form exhibits an X-ray powder diffraction pattern (XRPD) that is substantially similar to that of FIG. 4 (bottom trace).

It is contemplated that Compound 1 can exist in a variety of physical forms. For example, Compound 1 can be in solution, suspension, or in solid form. In certain embodiments, Compound 1 is in solid form. When Compound 1 is in solid form, said compound may be amorphous, crystalline, or a mixture thereof. Exemplary solid forms are described in more detail below.

In some embodiments, a form of Compound 1 can be substantially free of impurities. As used herein, the term “substantially free of impurities” means that the compound contains no significant amount of extraneous matter. Such extraneous matter may include different forms of Compound 1, residual solvents, or any other impurities that may result from the preparation of, and/or isolation of, Compound 1. In certain embodiments, at least about 95% by weight of a form of Compound 1 is present. In still other embodiments, at least about 99% by weight of a form of Compound 1 is present.

According to one embodiment, a form of Compound 1 is present in an amount of at least about 97, 97.5, 98.0, 98.5, 99, 99.5, 99.8 weight percent where the percentages are based on the total weight of the composition. According to another embodiment, a form of Compound 1 contains no more than about 3.0 area percent HPLC of total organic impurities and, in certain embodiments, no more than about 1.5 area percent HPLC total organic impurities relative to the total area of the HPLC chromatogram. In other embodiments, a form of Compound 1 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of Compound 1 is also meant to include all tautomeric forms of Compound 1. Additionally, structures depicted here are also meant to include compounds that differ only in the presence of one or more isotopically enriched atoms. For example, compounds can have the present structure except for the replacement of hydrogen by deuterium or tritium, or the replacement of a carbon by a ¹³C- or ¹⁴C-enriched carbon.

It has been found that Compound 1 can exist in a variety of solid forms. Exemplary such forms include polymorphs such as those described herein.

As used herein, the term “polymorph” refers to the different crystal structures into which a compound, or a salt or solvate thereof, can crystallize.

In certain embodiments, Compound 1 is a crystalline solid. In other embodiments, Compound 1 is a crystalline solid substantially free of amorphous Compound 1. As used herein, the term “substantially free of amorphous Compound 1” means that the compound contains no significant amount of amorphous Compound 1. In certain embodiments, at least about 95% by weight of crystalline Compound 1 is present. In still other embodiments, at least about 99% by weight of crystalline Compound 1 is present.

It has been found that Compound 1 can exist in distinct polymorphic forms. In certain embodiments, a polymorphic form of Compound 1 can be referred to herein as Form C. In certain embodiments, a polymorphic form of Compound 1 can be referred to herein as Form A.

In some embodiments, Compound 1 is amorphous. In some embodiments, Compound 1 is amorphous, and is substantially free of crystalline Compound 1.

### Form C of Compound 1

As described above, in one aspect, Form C of Compound 1 can be a crystalline solid:

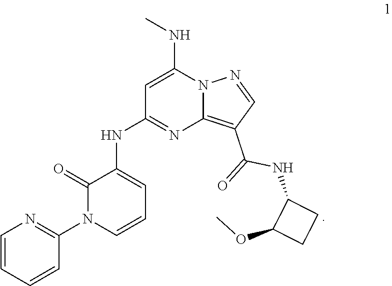 [SMILES: CNc1cc(Nc2cccn(-c3ccccn3)c2=O)nc2c(C(=O)N[C@@H]3CC[C@H]3OC)cnn12]

In some embodiments, Form C exhibits an X-ray powder diffraction pattern (XRPD) that is substantially similar to that of FIG. 16 (bottom trace).

In some aspects a crystalline Form C of Compound 1 is provided:

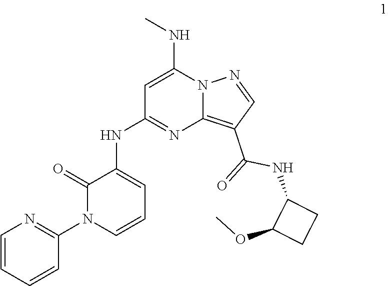 [SMILES: CNc1cc(Nc2cccn(-c3ccccn3)c2=O)nc2c(C(=O)N[C@@H]3CC[C@H]3OC)cnn12]

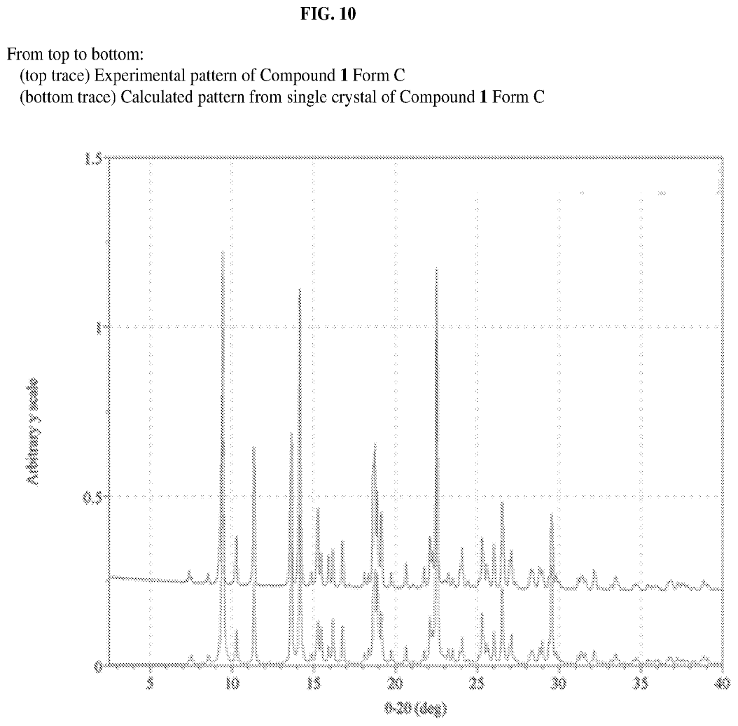

comprising an XRPD pattern substantially as shown in FIG. 10 (top trace).

In another aspect Form C of Compound 1 that is crystalline has an XRPD:

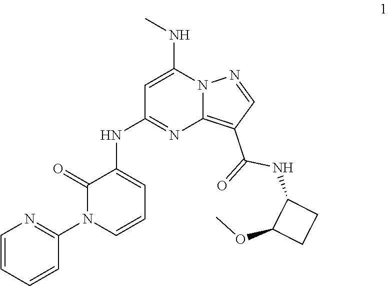 [SMILES: CNc1cc(Nc2cccn(-c3ccccn3)c2=O)nc2c(C(=O)N[C@@H]3CC[C@H]3OC)cnn12]

characterized by having at least 3 to 5 of the highest amplitude peaks of an X-ray powder diffraction pattern (XRPD) of Form C, that is substantially similar to that of FIG. 16 (bottom trace). In some embodiments, the XRPD has 3 of the highest amplitude peaks of the XRPD of FIG. 16 (bottom trace). In some embodiments, the XRPD has 4 of the highest amplitude peaks of the XRPD of FIG. 16 (bottom trace). In some embodiments, the XRPD has 5 of the highest amplitude peaks of the XRPD of FIG. 16 (bottom trace).

In another aspect Form C of Compound 1 that is crystalline has an XRPD:

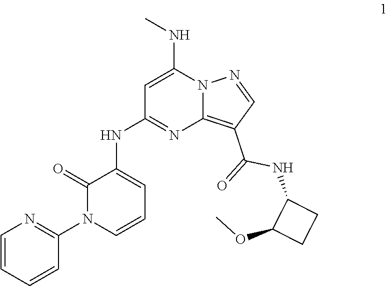 [SMILES: CNc1cc(Nc2cccn(-c3ccccn3)c2=O)nc2c(C(=O)N[C@@H]3CC[C@H]3OC)cnn12]

characterized by having at least 3 to 5 of the highest amplitude peaks of an X-ray powder diffraction pattern (XRPD) of Form C, that is substantially similar to that of FIG. 10 (top trace). In some embodiments, the XRPD has 3 of the highest amplitude peaks of the XRPD of FIG. 10 (top trace). In some embodiments, the XRPD has 4 of the highest amplitude peaks of the XRPD of FIG. 10 (top trace). In some embodiments, the XRPD has 5 of the highest amplitude peaks of the XRPD of FIG. 10 (top trace).

In one aspect a crystalline polymorph Form C of Compound 1:

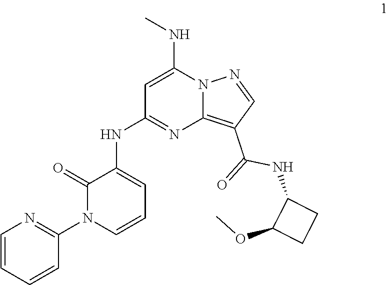 [SMILES: CNc1cc(Nc2cccn(-c3ccccn3)c2=O)nc2c(C(=O)N[C@@H]3CC[C@H]3OC)cnn12]

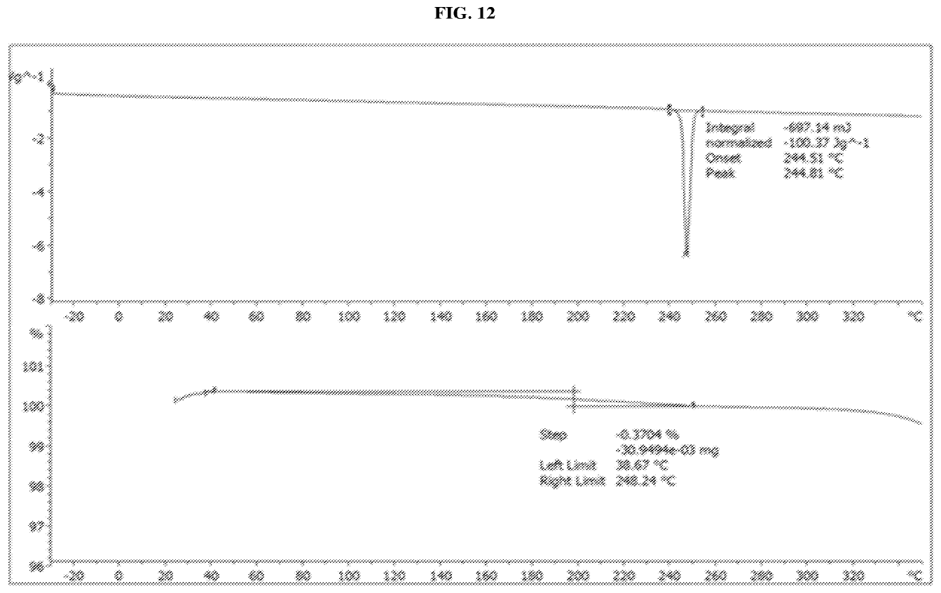

is characterized by a TGA analysis of mass loss between 165° C. and 175° C. In some embodiments, the TGA is substantially as shown in FIG. 12.

In one aspect a crystalline polymorph Form C of Compound 1:

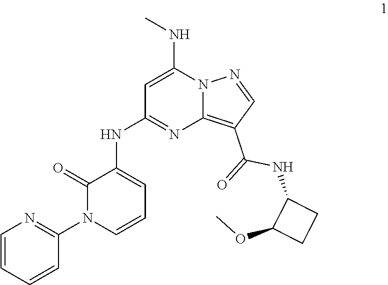 [SMILES: CNc1cc(Nc2cccn(-c3ccccn3)c2=O)nc2c(C(=O)N[C@@H]3CC[C@H]3OC)cnn12]

is characterized by a DSC comprising a peak onset at about 247° C.

In one aspect a crystalline polymorph Form C of Compound 1:

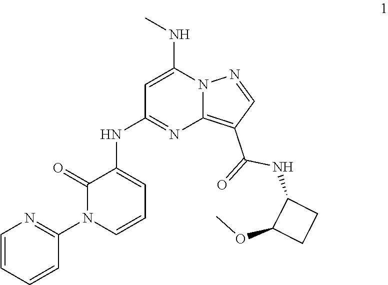 [SMILES: CNc1cc(Nc2cccn(-c3ccccn3)c2=O)nc2c(C(=O)N[C@@H]3CC[C@H]3OC)cnn12]

is characterized by a DSC substantially as shown in FIG. 12.

In one aspect a crystalline polymorph Form C of Compound 1:

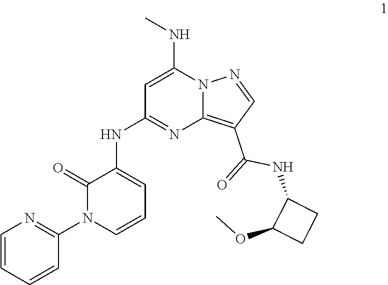 [SMILES: CNc1cc(Nc2cccn(-c3ccccn3)c2=O)nc2c(C(=O)N[C@@H]3CC[C@H]3OC)cnn12]

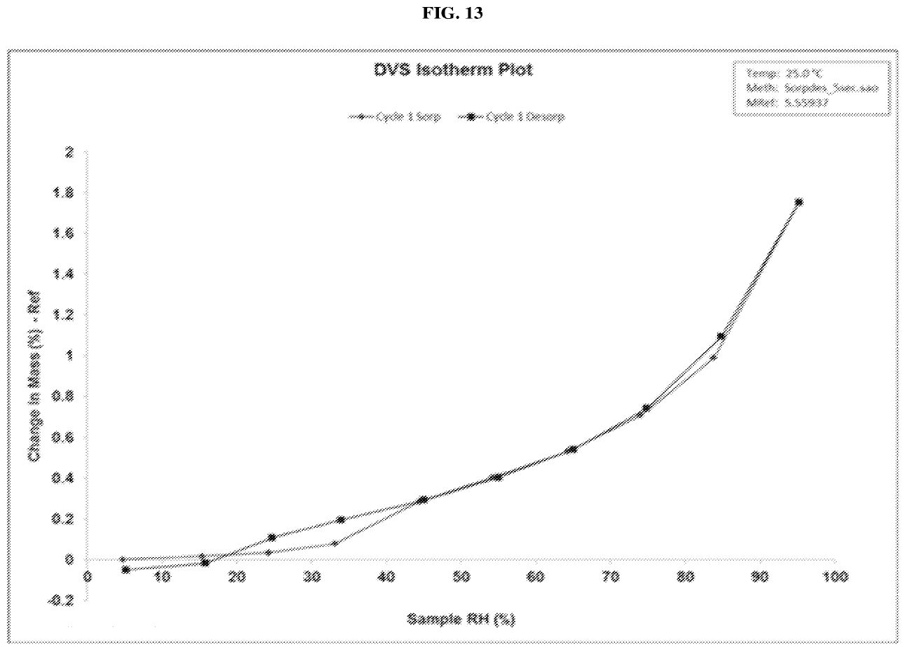

is characterized by a DVS isotherm substantially as shown in FIG. 13.

Methods for preparing Form C of Compound 1 are described infra.

### Form A of Compound 1

As described above, in one aspect, Form A of Compound 1 can be crystalline:

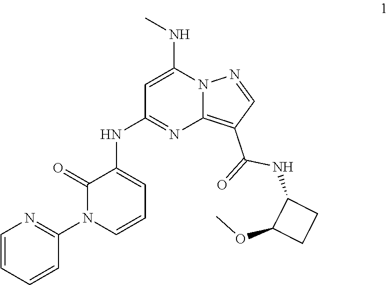 [SMILES: CNc1cc(Nc2cccn(-c3ccccn3)c2=O)nc2c(C(=O)N[C@@H]3CC[C@H]3OC)cnn12]

comprises an X-ray powder diffraction pattern (XRPD) that is substantially similar to that of FIG. 4 (bottom trace).

In some aspects, a crystalline Form A of Compound 1 is provided:

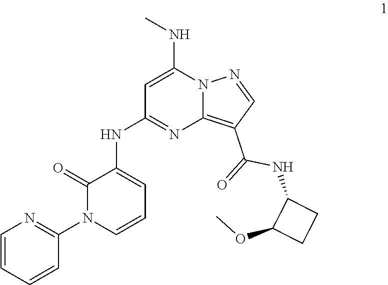 [SMILES: CNc1cc(Nc2cccn(-c3ccccn3)c2=O)nc2c(C(=O)N[C@@H]3CC[C@H]3OC)cnn12]

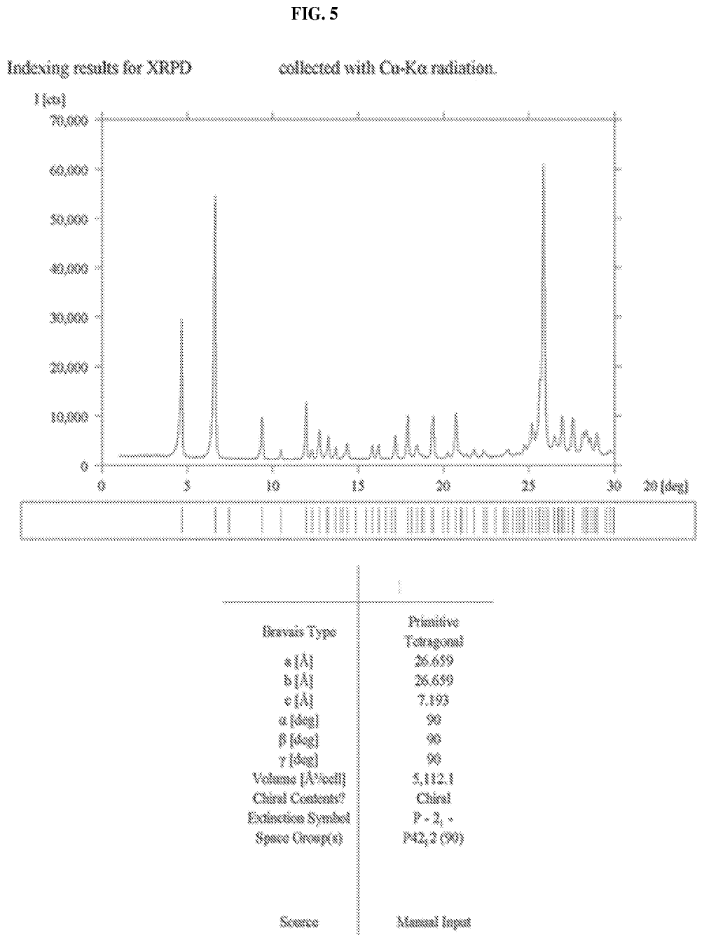

comprising an XRPD pattern substantially as shown in FIG. 5. In some embodiments, Form A is a hydrate. In some embodiments, Form A is a monohydrate. In some embodiments, Form A is a dihydrate. In some embodiments, Form A is a trihydrate.

In another aspect Form A of Compound 1 that is crystalline has an XRPD:

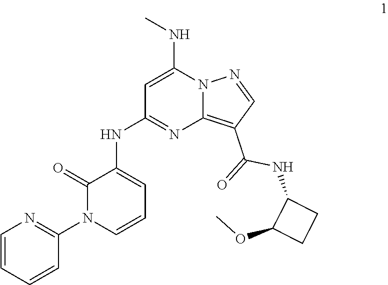 [SMILES: CNc1cc(Nc2cccn(-c3ccccn3)c2=O)nc2c(C(=O)N[C@@H]3CC[C@H]3OC)cnn12]

characterized by having at least 3 to 5 of the highest amplitude peaks of an X-ray powder diffraction pattern (XRPD) of Form A, that is substantially similar to that of FIG. 4 (bottom trace). In some embodiments, the XRPD has 3 of the highest amplitude peaks of the XRPD of FIG. 4 (bottom trace). In some embodiments, the XRPD has 4 of the highest amplitude peaks of the XRPD of FIG. 4 (bottom trace). In some embodiments, the XRPD has 5 of the highest amplitude peaks of the XRPD of FIG. 4 (bottom trace). In some embodiments, Form A is a hydrate. In some embodiments, Form A is a monohydrate. In some embodiments, Form A is a dihydrate. In some embodiments, Form A is a trihydrate.

In another aspect Form A of Compound 1 that is crystalline has an XRPD:

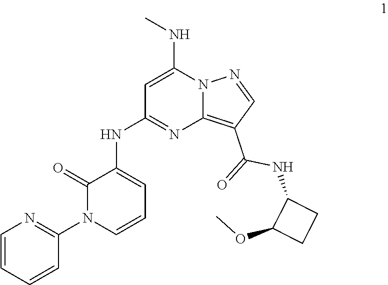 [SMILES: CNc1cc(Nc2cccn(-c3ccccn3)c2=O)nc2c(C(=O)N[C@@H]3CC[C@H]3OC)cnn12]

characterized by having at least 3 to 5 of the highest amplitude peaks of an X-ray powder diffraction pattern (XRPD) of Form A, that is substantially similar to that of FIG. 5. In some embodiments, the XRPD has 3 of the highest amplitude peaks of the XRPD of FIG. 5. In some embodiments, the XRPD has 4 of the highest amplitude peaks of the XRPD of FIG. 5. In some embodiments, the XRPD has 5 of the highest amplitude peaks of the XRPD of FIG. 5. In some embodiments, Form A is a hydrate. In some embodiments, Form A is a monohydrate. In some embodiments, Form A is a dihydrate. In some embodiments, Form A is a trihydrate.

In one aspect a crystalline polymorph Form A of Compound 1:

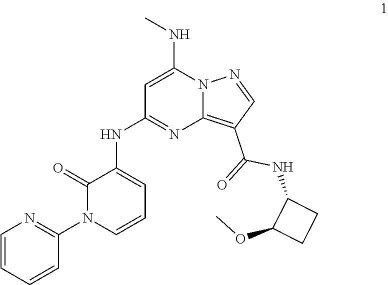 [SMILES: CNc1cc(Nc2cccn(-c3ccccn3)c2=O)nc2c(C(=O)N[C@@H]3CC[C@H]3OC)cnn12]

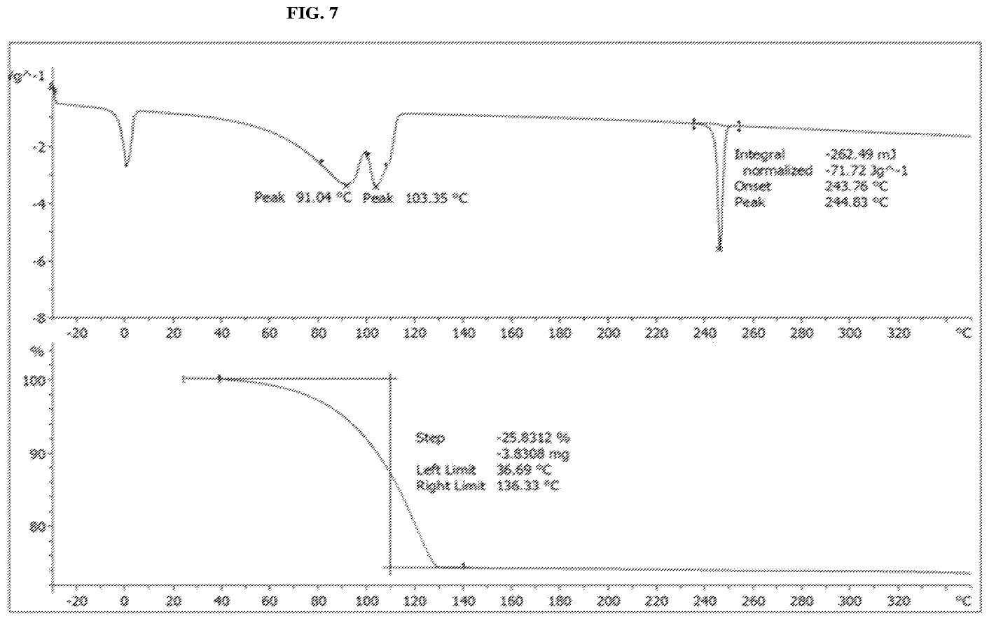

is characterized by a TGA analysis of mass loss between 65° C. and 130° C. In some embodiments, the TGA is substantially as shown in FIG. 7.

In one aspect a crystalline polymorph Form A of Compound 1:

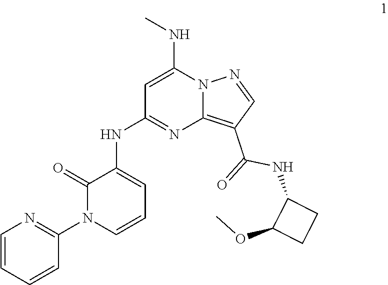 [SMILES: CNc1cc(Nc2cccn(-c3ccccn3)c2=O)nc2c(C(=O)N[C@@H]3CC[C@H]3OC)cnn12]

is characterized by a DSC comprising a peak onset at about 91° C., 103° C., and 245° C.

In one aspect a crystalline polymorph Form A of Compound 1:

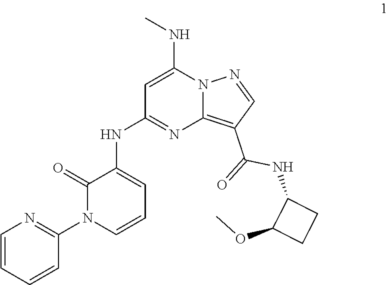 [SMILES: CNc1cc(Nc2cccn(-c3ccccn3)c2=O)nc2c(C(=O)N[C@@H]3CC[C@H]3OC)cnn12]

is characterized by a DSC substantially as shown in FIG. 7.

Methods for preparing Form A of Compound 1 are described infra.

In some embodiments, Compound 1:

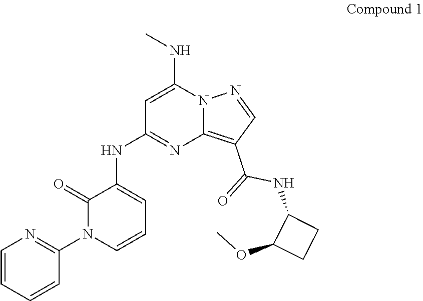 [SMILES: CNc1cc(Nc2cccn(-c3ccccn3)c2=O)nc2c(C(=O)N[C@@H]3CC[C@H]3OC)cnn12]

can be crystalline.

In some embodiments, a solid form of Compound 1 can be substantially free of amorphous Compound 1.

In some embodiments, a solid form of Compound 1 can be substantially free of impurities.

In some embodiments, a composition can include Compound 1 and a pharmaceutically acceptable carrier or excipient.

In some embodiments, a method of inhibiting or preventing the accumulation of A2E in a patient can include administering to said patient Compound 1 or composition thereof.

### Salt Forms of Compound 1

In some embodiments, an acid and Compound 1 are ionically bonded to form a salt, described below. It is contemplated that a salt can exist in a variety of physical forms. For example, a salt can be in solution, suspension, or in solid form. In certain embodiments, a salt is in solid form. When a salt is in solid form, said compounds may be amorphous, crystalline, or a mixture thereof. Exemplary such solid forms of a salt are described in more detail below. In some embodiments, a suitable acid is methanesulfonic acid. In some embodiments, a method can include making a mesylate salt of Compound 1. In certain embodiments, the mesylate salt of Compound 1 is crystalline.

In some embodiments, a suitable acid is benzenesulfonic acid. In some embodiments, a method can include making a besylate salt of Compound 1. In certain embodiments, the besylate salt of Compound 1 crystalline.

In some embodiments, a suitable acid is sulfuric acid. In some embodiments, a method can include making a sulfate salt of Compound 1. In certain embodiments, the sulfate salt of Compound 1 is crystalline.

In some embodiments, a suitable acid is p-toluenesulfonic acid. In some embodiments, a method can include making a tosylate salt of Compound 1. In certain embodiments, the tosylate salt of Compound 1 is crystalline.

In some embodiments, a suitable acid is hydrochloric acid. In some embodiments, a method can include making a hydrochloride salt of Compound 1. In certain embodiments, the hydrochloric salt of Compound 1 is crystalline.

In some embodiments, a suitable acid is oxalic acid. In some embodiments, a method can include making an oxalate salt of Compound 1. In certain embodiments, the oxalate salt of Compound 1 is crystalline.

In some embodiments, a suitable acid is phosphoric acid. In some embodiments, a method can include making a phosphate salt of Compound 1. In certain embodiments, the phosphate salt of Compound 1 is crystalline.

In some embodiments, a suitable acid is tartaric acid. In some embodiments, a method can include making a tartrate salt of Compound 1. In certain embodiments, the tartrate salt of Compound 1 is crystalline.

In some embodiments, a suitable acid is isethionic acid. In some embodiments, a method can include making an isethionate salt of Compound 1. In certain embodiments, the isethionate salt of Compound 1 is crystalline.

In some embodiments, a suitable acid is aspartic acid. In some embodiments, a method can include making an aspartate salt of Compound 1. In certain embodiments, the aspartate salt of Compound 1 is crystalline.

In some embodiments, a suitable acid is malonic acid. In some embodiments, a method can include making a malonate salt of Compound 1. In certain embodiments, the malonate salt of Compound 1 is crystalline.

A suitable solvent, used as a solvent or in a slurry for the methods of preparing the polymorph crystalline forms of Compound 1 may be any solvent system (e.g., one solvent or a mixture of solvents) in which Compound 1 and/or an acid are soluble, or are at least partially soluble.

Examples of suitable solvents can include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, the solvent is one or more organic alcohols. In some embodiments, the solvent is chlorinated. In some embodiments, the solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethylformamide, dimethylsulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, the suitable solvent is MTBE.

In some embodiments, a suitable solvent is ethyl acetate. In some embodiments, a suitable solvent is a mixture of methanol and methylene chloride. In some embodiments, a suitable solvent is a mixture of acetonitrile and water. In certain embodiments, a suitable solvent is methyl acetate, isopropyl acetate, acetone, or tetrahydrofuran. In certain embodiments, a suitable solvent is diethylether. In certain embodiments, a suitable solvent is water. In certain embodiments, a suitable solvent is methyl ethyl ketone. In certain embodiments, a suitable solvent is toluene.

In some embodiments, a method for preparing a salt compound of the general formula X (wherein Formula X comprises Compound 1 in salt form) can include one or more steps of removing a solvent and adding a solvent. In some embodiments, an added solvent is the same as the solvent removed. In some embodiments, an added solvent is different from the solvent removed. Means of solvent removal are known in the synthetic and chemical arts and include, but are not limited to, any of those described herein and in the Exemplification.

In some embodiments, a method for preparing a salt compound of the general formula X comprises one or more steps of heating or cooling a preparation.

In some embodiments, a method for preparing a salt compound of the general formula X comprises one or more steps of agitating or stirring a preparation.

In some embodiments, a method for preparing a salt compound of the general formula X comprises a step of adding a suitable acid to a solution or slurry of Compound 1.

In some embodiments, a method for preparing a salt compound of the general formula X comprises a step of heating.

In certain embodiments, a salt compound of formula X precipitates from the mixture. In another embodiment, a salt compound of formula X crystallizes from the mixture. In other embodiments, a salt compound of formula X crystallizes from solution following seeding of the solution (i.e., adding crystals of a salt compound of formula X to the solution).

A salt compound of formula X can precipitate out of the reaction mixture, or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a salt compound of formula X is optionally isolated. It will be appreciated that a salt compound of formula X may be isolated by any suitable physical means known to one of ordinary skill in the art. In certain embodiments, precipitated solid salt compound of formula X is separated from the supernatant by filtration. In other embodiments, precipitated solid salt compound of formula X is separated from the supernatant by decanting the supernatant.

In certain embodiments, a salt compound of formula X is separated from the supernatant by filtration.

In certain embodiments, an isolated salt compound of formula X is dried in air. In other embodiments, isolated salt compound of formula X is dried under reduced pressure, optionally at elevated temperature.

### Uses of Compounds and Pharmaceutically Acceptable Compositions Thereof

The present Compound 1 has a selectivity for kinase inhibition as a TYK2 inhibitor. Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

Crystalline compounds described herein can be used for the treatment, prevention, and/or reduction of a risk of autoimmune diseases, inflammatory diseases, bone diseases, metabolic diseases, neurological and neurodegenerative diseases, cancer, cardiovascular diseases, allergies and asthma, Alzheimer's disease, and hormone-related diseases. “Compound 1” or “the compound” may be formulated into a pharmaceutical formulation described infra for therapeutic use as described herein.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g. IFN-α, IFN-β, IFN-κ, IFN-δ, IFN-ε, IFN-τ, IFN-ω, and IFN-ζ (also known as limitin), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signaling pathway,” Cell (1992) 70:313; Stahl et al., “Association and activation of Jak-Tyk kinases by CNTF-LIF-OSM-IL-6β receptor components,” Science (1994) 263:92; Finbloom et al., “IL-10 induces the tyrosine phosphorylation of Tyk2 and Jak1 and the differential assembly of Stat1 and Stat3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoictic cells: similarities and differences in signal transduction with interleukin-4 and insulin,” J. Biol. Chem. (1995) 270:12286; Parham et al., “A receptor for the heterodimeric cytokine IL-23 is composed of IL-12Rβ1 and a novel cytokine receptor subunit, IL-23R,” J. Immunol. (2002) 168:5699. The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. Ducrr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behçet's disease. Cho et al., “Genomics and the multifactorial nature of human auto-immune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MHC class I, IL10, and IL23R-IL12RB2 regions associated with Beheet's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAP1,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11.

Decreased TYK2 activity leads to protection of joints from collagen antibody-induced arthritis, a model of human rheumatoid arthritis. Mechanistically, decreased Tyk2 activity reduced the production of Tₕ1/Tₕ17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupus Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association of NCF2, IKZF1, IRF8, IFIH1, and TYK2 with Systemic Lupus Erythematosus,” PLoS Genetics (2011) 7(10):e1002341.

TYK2 has been shown to play an important role in maintaining tumour surveillance and TYK2 knockout mice showed compromised cytotoxic T cell response, and accelerated tumour development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects. Simma et al. “Identification of an Indispensable Role for Tyrosine Kinase 2 in CTL-Mediated Tumour Surveillance,” Cancer Res. (2009) 69:203-211.

However, paradoxically to the decreased tumour surveillance observed by Simma et al., studies in T-cell acute lymphoblastic leukaemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective inhibition of TYK2 has been suggested as a suitable target for patients with IL-10 and/or BCL2-addicted tumours, such as 70% of adult T-cell leukaemia cases. Fontan et al. “Discovering What Makes STAT Signaling TYK in T-ALL,” Cancer Disc. (2013) 3:494-496.

TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in post-mortem brains of Alzheimer's patients. Wan et al. “Tyk/STAT3 Signaling Mediates β-Amyloid-Induced Neuronal Cell Death: Implications in Alzheimer's Disease,” J. Neurosci. (2010) 30(20):6873-6881.

Inhibition of JAK-STAT signaling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signaling promotes hair growth,” Sci. Adv. (2015) 1(9):e1500973.

Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, a pharmaceutical composition, formulation, or unit dosage form can include the Compound 1 inhibitor of TYK2 which shows selectivity over JAK2.

The activity of Compound 1 can be utilized as an inhibitor of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 inhibitor include those described and disclosed in, e.g., each of which is herein incorporated by reference in its entirety.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Compound 1 is an inhibitor of TYK2 and is therefore useful for treating one or more disorders associated with activity of TYK2 or mutants thereof. Thus, in certain embodiments, a method for treating a TYK2-mediated disorder can include the step of administering to a patient in need thereof a pharmaceutical composition, formulation, or unit dosage form described herein, comprising Compound 1, or pharmaceutically acceptable salt, or hydrate thereof.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

In some embodiments, a method for treating one or more disorders, wherein the disorders are selected from autoimmune disorders, inflammatory disorders, proliferative disorders, endocrine disorders, neurological disorders, and disorders associated with transplantation, said method can include administering to a patient in need thereof, a pharmaceutical composition comprising an effective amount of a pharmaceutical composition, formulation, or unit dosage form comprising Compound 1 as described herein.

In some embodiments, the disorder is an autoimmune disorder. In some embodiments the disorder is selected from type 1 diabetes, cutaneous lupus erythematosus, systemic lupus erythematosus, multiple sclerosis, psoriasis, Behçet's disease, POEMS syndrome, Crohn's disease, ulcerative colitis, and inflammatory bowel disease.

In some embodiments, the disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disorder is a proliferative disorder. In some embodiments, the proliferative disorder is a hematological cancer. In some embodiments the proliferative disorder is a leukaemia. In some embodiments, the leukaemia is a T-cell leukaemia. In some embodiments the T-cell leukaemia is T-cell acute lymphoblastic leukaemia (T-ALL). In some embodiments the proliferative disorder is polycythemia vera, myelofibrosis, essential or thrombocytosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signaling. In some embodiments the disorder is associated with type I interferon signaling. In some embodiments the disorder is associated with IL-10 signaling. In some embodiments the disorder is associated with IL-12 signaling. In some embodiments the disorder is associated with IL-23 signaling.

Formulations comprising Compound 1 are also useful in the treatment of inflammatory or allergic conditions of the skin, for example psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, cutaneous lupus erythematosus, systemic lupus erythematosus, Pemphigus vulgaris, Pemphigus foliaceus, paraneoplastic pemphigus, epidermolysis bullosa acquisita, acne vulgaris, and other inflammatory or allergic conditions of the skin.

Formulations comprising Compound 1 may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minimal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods described herein is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods described herein is a Tₕ1 or Tₕ17 mediated disease. In some embodiments the Tₕ17 mediated disease is selected from cutaneous lupus erythematosus, Systemic lupus erythematosus, Multiple sclerosis, and inflammatory bowel disease (including Crohn's disease or ulcerative colitis).

In some embodiments the inflammatory disease which can be treated according to the methods described herein is selected from Sjogren's syndrome, psoriasis, psoriatic arthritis, irritable bowel disease, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, a formulation can include Compound 1 according to the definitions herein, for the preparation of a medicament for the treatment of an autoimmune disorder, an inflammatory disorder, or a proliferative disorder, or a disorder commonly occurring in connection with transplantation.

### Combination Therapies

Depending upon the particular condition, or disease, to be treated, additional therapeutic agents, which are normally administered to treat that condition, may be administered in combination with formulations comprising Compound 1 described herein. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations described herein may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinirole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies described herein, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents as described herein. For example, a combination described herein may be administered with another therapeutic agent simultaneously or sequentially in separate unit dosage forms or together in a single unit dosage form.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, a composition can include Compound 1 and one or more additional therapeutic agents. The therapeutic agent may be administered together with Compound 1 may be administered prior to or following administration of the additional therapeutic agent. Suitable therapeutic agents are described in further detail below. In certain embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, a method of treating an inflammatory disease, disorder or condition by administering to a patient in need thereof a formulation can include Compound 1 as described herein and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colerys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbutcrol acetate (Maxair®), terbutaline sulfate (Brethaire®), salmeterol xinafoate (Screvent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, a method of treating rheumatoid arthritis can include administering to a patient in need thereof a formulation comprising Compound 1 and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kincret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, a method of treating osteoarthritis can include administering to a patient in need thereof a formulation comprising Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, a method of treating cutaneous lupus erythematosus or systemic lupus erythematosus can include administering to a patient in need thereof a formulation comprising Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, a method of treating Crohn's disease, ulcerative colitis, or inflammatory bowel disease can include administering to a patient in need thereof a formulation comprising Compound 1 and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, a method of treating asthma can include administering to a patient in need thereof a formulation comprising Compound 1 and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-Bid®, Uniphyl®, Theo-24®) and aminophylline, and IgE antibodies such as omalizumab (Xolair®).

In some embodiments, a method of treating COPD can include administering to a patient in need thereof a formulation comprising Compound 1 and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-Bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, a method of treating a hematological malignancy can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, a method of treating a solid tumour can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, a method of treating a hematological malignancy can include administering to a patient in need thereof a formulation comprising Compound 1 and a Hedgehog (Hh) signaling pathway inhibitor. In some embodiments, the hematological malignancy is DLBCL (Ramirez et al “Defining causative factors contributing to the activation of hedgehog signaling in diffuse large B-cell lymphoma” Leuk. Res. (2012), published online July 17, and incorporated herein by reference in its entirety).

In another embodiment, a method of treating diffuse large B-cell lymphoma (DLBCL) can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, a method of treating multiple myeloma can include administering to a patient in need thereof a compound of a formulation comprising Compound 1 and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, a method of treating or lessening the severity of a disease can include administering to a patient in need thereof a formulation comprising Compound 1 and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukaemia, acute lymphocytic leukaemia, B-cell prolymphocytic leukaemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukaemia, or lymphomatoid granulomatosis, breast cancer, prostate cancer, or cancer of the mast cells (e.g., mastocytoma, mast cell leukaemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, ostcomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, scleroderma, mycosis fungoides, and acute inflammatory responses (such as acute respiratory distress syndrome and ischemia/reperfusion injury).

In another embodiment, a method of treating or lessening the severity of a disease can include administering to a patient in need thereof a formulation comprising Compound 1 disclosed herein, and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukaemia (CML), chronic lymphocytic leukaemia (CLL), liver disease, pathologic immune conditions involving T cell activation, a cardiovascular disorder, and a CNS disorder.

In another embodiment, a method of treating or lessening the severity of a disease can include administering to a patient in need thereof a formulation comprising Compound 1 disclosed herein, and a PI3K inhibitor, wherein the disease is selected from benign or malignant tumour, carcinoma or solid tumour of the brain, kidney (e.g., renal cell carcinoma (RCC)), liver, adrenal gland, bladder, breast, stomach, gastric tumours, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumour of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukaemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleredoma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g., including idiopathic nephrotic syndrome or minimal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, a method of treating or lessening the severity of a disease can include administering to a patient in need thereof a formulation comprising Compound 1 disclosed herein, and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukaemia, diffuse large B-cell lymphoma, Hodgkin's disease, small-cell lung cancer, non-small-cell lung cancer, myelodysplastic syndrome, lymphoma, a hematological neoplasm, or solid tumour.

In some embodiments, a method of treating or lessening the severity of a disease, can include administering to a patient in need thereof a TYK2 pseudokinase (JH2) domain binding compound and a TYK2 kinase (JH1) domain binding compound. In some embodiments, the disease is an autoimmune disorder, an inflammatory disorder, a proliferative disorder, an endocrine disorder, a neurological disorder, or a disorder associated with transplantation. In some embodiments the JH2 is binding Compound 1. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

Compound 1 compositions may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compound 1 is preferably formulated in unit dosage form for ease of administration and uniformity of dosage. The expression “unit dosage form” as used herein refers to a physically discrete unit of agent appropriate for the patient to be treated. It will be understood, however, that the total daily usage of the compound and compositions described herein will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

Pharmaceutically acceptable compositions can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compound may be administered orally or parenterally at dosage levels of about 0.01 mg/kg to about 50 mg/kg and preferably from about 0.01 mg/kg to about 5 mg/kg of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a Compound 1, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of Compound 1 release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing Compound 1 with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound can also be in micro-encapsulated form with one or more excipients as noted above. In some embodiments, hydroxypropyl methyl cellulose (HMPC) capsules encapsulate the composition or formulation. In some embodiments, the capsules are size 2 hard Swedish orange HPMC capsules. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a formulation comprising Compound 1 include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops can also be formulated. Additionally, transdermal patches can be used, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, a method of inhibiting protein kinase activity in a biological sample can include the step of contacting said biological sample with a formulation comprising Compound 1 described herein.

According to another embodiment, a method of inhibiting TYK2, or a mutant thereof, activity in a biological sample can include the step of contacting said biological sample with Compound 1 described herein, or a composition comprising said compound. In certain embodiments, a method of irreversibly inhibiting TYK2, or a mutant thereof, activity in a biological sample can include the step of contacting said biological sample with a formulation comprising Compound 1 described herein.

In another embodiment, a method can include selectively inhibiting TYK2 over one or more of JAK1, JAK2, and JAK3. In some embodiments, a formulation comprising Compound 1 described herein is more than 2-fold selective over JAK1/2/3. In some embodiments, a compound described herein is more than 5-fold selective over JAK1/2/3. In some embodiments, the compound described herein is more than 10-fold selective over JAK1/2/3. In some embodiments, the compound described herein is more than 50-fold selective over JAK1/2/3. In some embodiments, the compound described herein is more than 100-fold selective over JAK1/2/3.

The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of TYK2 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment relates to a method of inhibiting protein kinase activity in a patient comprising the step of administering to said patient a formulation comprising Compound 1 described herein.

According to another embodiment, a method of inhibiting activity of TYK2, or a mutant thereof, in a patient can include the step of administering to said patient a formulation comprising Compound 1 described herein. According to certain embodiments, a method of reversibly or irreversibly inhibiting one or more of TYK2, or a mutant thereof, activity in a patient can include the step of administering to said patient a formulation comprising Compound 1 described herein. In other embodiments, a method for treating a disorder mediated by TYK2, or a mutant thereof, in a patient in need thereof, can include the step of administering to said patient a formulation comprising Compound 1 described herein. Such disorders are described in detail herein.

Depending upon the particular condition, or disease, to be treated, additional therapeutic agents that are normally administered to treat that condition, may also be present in the compositions described herein. As used herein, additional therapeutic agents that are normally administered to treat a particular disease, or condition, are known as “appropriate for the disease, or condition, being treated.”

A formulation comprising Compound 1 described herein may also be used in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase TT inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination described herein comprising a chemotherapeutic agent which is an aromatase inhibitor is particularly useful for the treatment of hormone receptor positive tumours, such as breast tumours.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination described herein comprising a chemotherapeutic agent which is an antiestrogen is particularly useful for the treatment of estrogen receptor positive tumours, such as breast tumours.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g., under the trademark Carboplat™. Oxaliplatin can be administered, e.g., in the form as it is marketed, e.g., under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; isis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds described herein can be found in WO2008039218 and WO2011090760, the entirety of which are incorporated herein by reference.

Further examples of SYK inhibitory compounds, and conditions treatable by such compounds in combination with compounds described herein can be found in WO2003063794, WO2005007623, and WO2006078846, the entirety of which are incorporated herein by reference.

Further examples of PI3K inhibitory compounds, and conditions treatable by such compounds in combination with compounds described herein can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds described herein can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (Thalomid™) and TNP-470.

Examples of proteasome inhibitors useful for use in combination with a formulation comprising Compound 1 described herein include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g., inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR), and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms,” such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds described herein can be used in combination with standard leukaemia therapies, especially in combination with therapies used for the treatment of AML. In particular, a formulation comprising Compound 1 described herein can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, a method of treating AML associated with an ITD and/or D835Y mutation, can include administering a formulation comprising Compound 1 described herein together with a one or more FLT3 inhibitors. In some embodiments, the FLT3 inhibitors are selected from quizartinib (AC220), a staurosporine derivative (e.g., midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumour cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The formulation comprising Compound 1 described herein is also useful as a co-therapeutic compound for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A formulation comprising Compound 1 described herein may be mixed with the other drug substance in a fixed pharmaceutical composition or it may be administered separately, before, simultaneously with or after the other drug substance. Accordingly, a combination of a formulation comprising Compound 1 described herein with an anti-inflammatory, bronchodilatory, antihistamine or anti-tussive drug substance, said compound described herein and said drug substance being in the same or different pharmaceutical composition can be prepared.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds described herein with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g., Patents International (e.g., IMS World Publications).

A formulation comprising Compound 1 described herein may also be used in combination with known therapeutic processes, for example, the administration of hormones or radiation. In certain embodiments, a provided compound is used as a radiosensitizer, especially for the treatment of tumours which exhibit poor sensitivity to radiotherapy.

A formulation comprising Compound 1 described herein can be administered alone or in combination with one or more other therapeutic compounds, possible combination therapy taking the form of fixed combinations or the administration of Compound 1 as a formulation and one or more other therapeutic compounds being staggered or given independently of one another, or the combined administration of fixed combinations and one or more other therapeutic compounds. Compound 1 can besides or in addition be administered especially for tumour therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumour regression, or even chemopreventive therapy, for example in patients at risk.

Those additional agents may be administered separately from an inventive compound-containing composition, as part of a multiple dosage regimen. Alternatively, those agents may be part of a single dosage form, mixed together with Compound 1 in a single formulation or composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, Compound 1 may be administered with another therapeutic agent simultaneously or sequentially in separate unit dosage forms or together in a single unit dosage form. Accordingly, the present invention provides a single unit dosage form comprising Compound 1, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions should be formulated so that a dosage of between 0.01-10 mg/kg body weight/day of a Compound 1 can be administered.

In those compositions which comprise an additional therapeutic agent, that additional therapeutic agent and Compound 1 may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions comprising Compound 1 will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compound 1 and pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound described herein are another embodiment. In some embodiments, a medicament can include at least Compound 1 formulated as described herein.

### Pharmaceutically Acceptable Compositions

The compositions described herein are administered using any amount and any route of administration effective for treating or lessening the severity of a disease described above. The exact amount required will vary from subject to subject, depending on the species, age, and general condition of the subject, the severity of the infection, the particular agent, its mode of administration, and the like. Compound 1 is preferably formulated in unit dosage form for ease of administration and uniformity of dosage, for example, as Form C. The expression “unit dosage form” as used herein refers to a physically discrete unit of agent appropriate for the patient to be treated. It will be understood, however, that the total daily usage of the compounds and compositions described herein will be decided by the attending physician within the scope of sound medical judgment. The specific effective dose level for any particular patient or organism (e.g., and feline, canine, bovine, equine, porcine, or aves) will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions described herein can be administered to humans and other animals orally, rectally, parenterally, intracisternally, intrathecally, transdermally, transmucosally, ophthalmically, via inhalation, intravaginally, intraperitoneally, topically (as by powders, ointments, or drops), buccally, intranasally, as an oral or nasal spray, or the like, depending on the severity of the disease being treated. In certain embodiments, the compounds described herein are administered orally or parenterally at dosage levels of about 0.01 mg/kg to about 50 mg/kg and for example from about 1 mg/kg to about 25 mg/kg of subject body weight per day, one or more times a day, to obtain the desired therapeutic effect.

A unit dosage form described herein can be formulated for oral administration. Pharmaceutical compositions/formulations that are suitable for oral administration can be provided as discrete dosage forms, such as, but not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In some embodiments, such dosage forms contain predetermined amounts of active ingredients, and may be prepared by methods of pharmacy known to those skilled in the art. See generally, Remington's Pharmaceutical Sciences, 18^{th} ed., Mack Publishing, Easton Pa. (1990). As used herein, oral administration also includes buccal, lingual, and sublingual administration.

In some embodiments, the formulation further comprises one or more pharmaceutically acceptable excipients or carriers.

A person of ordinary skill would recognize that pharmaceutical formulation ingredients may serve multiple purposes within a formulation. Accordingly, a person of ordinary skill would recognize that certain formulation components may be classified according to multiple functions (e.g., a component may be both a filler and a binder).

In some embodiments, a unit dosage form provided herein are prepared by combining the active ingredients in an intimate admixture with one or more pharmaceutically acceptable excipients or carriers, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide, according to conventional pharmaceutical compounding techniques. Excipients or carriers can take a wide variety of forms depending on the form of preparation desired for administration. For example, excipients or carriers suitable for use in oral liquid or aerosol dosage forms include, but are not limited to, water, glycols, oils, alcohols, flavoring agents, preservatives, and coloring agents. Examples of excipients or carriers suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In some embodiments, the active ingredient, such as a solid form of Compound 1 or a pharmaceutically acceptable salt thereof, is incorporated into the pharmaceutical composition as spray-dried powder or granules. The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See commonly owned European Patent Applications Nos. 0 901 786, 1 027 886, 1 027 887, 1 027 888, and commonly owned PCT Applications Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

The spray-dried powder or granules generally include the active compound in combination with a polymer such as a concentration-enhancing polymer. One class of polymers suitable for use herein comprises non-ionizable (neutral) non-cellulosic polymers. Exemplary polymers include vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers; and polyoxyethylene-polyoxypropylene copolymers.

Exemplary neutral non-cellulosic polymers are comprised of vinyl copolymers of at least one hydrophilic, hydroxyl-containing repeat unit and at least one hydrophobic, alkyl- or aryl-containing repeat unit. Such neutral vinyl copolymers are termed “amphiphilic hydroxyl-functional vinyl copolymers.” Amphiphilic hydroxyl-functional vinyl copolymers are believed to provide high concentration enhancements due to the amphiphilicity of these copolymers which provide both sufficient hydrophobic groups to interact with the hydrophobic, low-solubility drugs and also sufficient hydrophilic groups to have sufficient aqueous solubility for good dissolution. The copolymeric structure of the amphiphilic hydroxyl-functional vinyl copolymers also allows their hydrophilicity and hydrophobicity to be adjusted to maximize performance with a specific low-solubility drug.

Another class of polymers suitable for use herein comprises ionizable non-cellulosic polymers. Exemplary polymers include carboxylic acid-functionalized vinyl polymers, such as the carboxylic acid functionalized polymethacrylates and carboxylic acid functionalized polyacrylates such as the EUDRAGIT™ series manufactured by Rohm Tech Inc., of Malden, Mass.; amine-functionalized polyacrylates and polymethacrylates; proteins such as gelatin and albumin; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGIT™ series, which are copolymers of methacrylates and acrylates.

An additional class of polymers comprises ionizable and neutral (or non-ionizable) cellulosic polymers with at least one ester- and/or ether-linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate-substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous-insoluble. Examples of hydrophobic substituent include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary nonionizable cellulosic polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

An exemplary class of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A particular class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester-linked ionizable substituents include carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Exemplary cellulosic polymers that are at least partially-ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A further subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose. Of these cellulosic polymers that are at least partially ionized at physiologically relevant pHs, for example, hydroxypropyl methyl cellulose acetate succinate, hydroxypropyl methyl cellulose phthalate, cellulose acetate phthalate, cellulose acetate trimellitate and carboxymethyl ethyl cellulose. In some embodiments, the polymer is hydroxypropyl methyl cellulose acetate succinate (HPMCAS).

Another class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “neutralized acidic cellulosic polymers” is meant any cellulosic “acidic polymer” in which a significant fraction of the “acidic moieties” or “acidic substituents” have been “neutralized.” By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKₐ of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.”

The amount of concentration-enhancing polymer relative to the amount of drug (Compound 1) present in the spray-dried dispersions depends on the drug and concentration-enhancing polymer and may vary widely from a drug-to-polymer weight ratio of 0.01 to 5. However, in most cases, except when the drug dose is quite low, e.g., 25 mg or less, it is preferred that the drug-to-polymer ratio is greater than 0.05 and less than 2.5 and often the enhancement in drug concentration or relative bioavailability is observed at drug-to-polymer ratios of 1 or less or for some drugs even 0.2 or less. In cases where the drug dose is about 25 mg or less, the drug-to-polymer weight ratio may be significantly less than 0.05. In general, regardless of the dose, enhancements in drug concentration or relative bioavailability increase with decreasing drug-to-polymer weight ratio. However, due to the practical limits of keeping the total mass of a tablet, capsule or suspension low, it is often desirable to use a relatively high drug-to-polymer ratio as long as satisfactory results are obtained. The maximum drug:polymer ratio that yields satisfactory results varies from drug to drug and is best determined in the in dissolution tests described below.

A spray-dried solid as described herein can be a solid dispersion that contains a compound described herein and a pharmaceutically acceptable polymer. Certain compounds described herein generally have low aqueous solubility, and their absorption in vivo is dissolution-rate limited. A solid dispersion containing a compound can increase the compound solubility/dissolution, thereby improving the bioavailability of the compound.

The term “solid dispersion” herein refers to the dispersion of a pharmaceutically active ingredient, e.g., the compound described herein, in an inert polymer matrix at solid state. A solid dispersion can be prepared by methods well known in the art, e.g., spray-drying or hot-melt extrusion. The matrix can be either crystalline or amorphous. A solid dispersion contains a co-precipitate of a pharmaceutically active ingredient and one or more water-soluble polymers, in which the pharmaceutically active ingredient is dispersed uniformly within a polymer matrix formed from the polymers. The pharmaceutically active ingredient can be present in an amorphous state, a crystalline dispersed form, or a combination thereof. It can also be finely dispersed or dissolved as single molecules in the polymer matrix. The solid dispersion is typically prepared by a spray-drying method or a hot-melt extrusion method.

The method for preparing the solid dispersion includes steps of (i) mixing a compound described herein and a polymer in an organic solvent to provide a feeder solution and (ii) spray-drying the feeder solution through a nozzle as a fine spray into a chamber where the solvent is evaporated quickly to generate particles containing the compound and polymer. Following formation of a solid dispersion, the resulting spray-dried particle can undergo a secondary drying step to remove residual solvents. The secondary drying step can take place in a static dryer or an agitated dryer. Gas, humidified gas, vacuum can be applied to the secondary drying step and such application is useful in more rapidly removing residual solvents that remain in the spray-dried particle.

Any organic solvent that can easily dissolve or disperse the compound and the polymer described above can be used. Examples of the organic solvent include lower carbon-number alcohols, e.g., methanol, ethanol, propanol, and isopropanol; ketones, e.g., methylethyl ketone and butanone; and a combination thereof.

In some embodiments, the pharmaceutically acceptable excipients and carriers are selected from fillers, binders, diluents, disintegrants, glidants, and lubricants.

In some embodiments, a capsule or tablet can include a provided pharmaceutical composition in the form of a solid dosage form. In some embodiments, the composition can be in a capsule. In some embodiments, the composition can be in a tablet.

In certain embodiments, the dosage form is a tablet, wherein the tablet is manufactured using standard, art-recognized tablet processing procedures and equipment. In certain embodiments, the method for forming the tablets is direct compression of a powdered, crystalline and/or granular composition comprising a solid form provided herein, alone or in combination with one or more excipients or carriers, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the dosage form is a capsule, wherein the capsules may be manufactured using standard, art-recognized capsule processing procedures and equipment. In certain embodiments, soft gelatin capsules may be prepared in which the capsules contain a mixture comprising a solid form provided herein and vegetable oil or non-aqueous, water miscible materials, such as, for example, polyethylene glycol and the like. In certain embodiments, hard gelatin capsules may be prepared containing granules of solid forms provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In some embodiments, the pharmaceutical composition comprises one or more fillers. In certain embodiments, the filler is selected from ammonium aliginate, calcium carbonate, calcium lactate, calcium phosphate, calcium silicate, calcium sulfate, cellulose acetate, compressible sugar (e.g., lactose, glucose, and sucrose), corn starch, dextrates, erythritol, ethyl cellulose, glyceryl palmitostearate, isomalt, kaolin, magnesium carbonate, magnesium oxide, maltodextrin, medium-chain triglycerides, microcrystalline cellulose, pre-gelatinized starch, polydextrose, polymethacrylates, silicic acid, simethicone, sodium alginate, sodium chloride, sorbitol, starch, sugar spheres, sulfobutylether β-cyclodextrin, talc, tragacanth, trehalose, and xylitol, or a combination thereof.

In some embodiments, the filler is selected from talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some embodiments, the filler is microcrystalline cellulose. In some embodiments, the filler is lactose. In some embodiments, the filler is starch. In some embodiments, the filler is a combination of starch and lactose. In some embodiments, the filler is a combination of lactose and microcrystalline cellulose. In some embodiments, the filler is a combination of two or three components recited above. In some embodiments, the filler comprises at least microcrystalline cellulose, lactose, and mannitol.

In certain embodiments, dosage forms provided herein comprise one or more diluents. Diluents may be used, e.g., to increase bulk so that a practical size tablet or capsule is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-β-cyclodextrin, talc, tragacanth, trehalose, and xylitol.

In some embodiments, the pharmaceutical composition comprises one or more binders. Binders may be used, e.g., to impart cohesive qualities to a tablet or a capsule, and thus ensure that the formulation remains intact after compression. In some embodiments, the binder is selected from acacia gum, agar, alginic acid, calcium carbonate, calcium lactate, carbomers (e.g., acrylic acid polymer, carboxy polymethylene, polyacrylic acid, carboxyvinyl polymer), carboxymethylcellulose sodium, carrageenan, cellulose acetate phthalate, ceratonia, chitosan, copovidone, corn starch, cottonseed oil, dextrates, dextrin, dextrose, ethylcellulose, gelatin, glyceryl behenate, guar gum, hydrogenated vegetable oil type I, hydroxyethylcellulose, hydroxyethylmethyl cellulose, hydroxypropyl cellulose, hydroxypropyl methylcellulose, hypromellose, inulin, lactose, magnesium aluminum silicate, maltodextrin, maltose, methylcellulose, microcrystalline cellulose, pectin, poloxamer, polycarbohil, polydextrose, polyethylene oxide, polymetharylates, polyvinylpyrrolidone, pre-gelatinized starch, povidone, sodium alginate, starch, stearic acid, sucrose, tricaprylin, vitamin E polyethylene glycol succinate, and zein.

Suitable binders include, but are not limited to, starch (including potato starch, corn starch, and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone (PVP), cellulosic polymers (including hydroxypropyl cellulose (HPC), hydroxypropylmethylcellulose (HPMC), methyl cellulose, ethyl cellulose, hydroxyethyl cellulose (HEC), carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein.

Suitable forms of microcrystalline cellulose include, but are not limited to, the materials sold as AVICEL-PH-101, AVICEL-PH-103 AVICEL RC-581, AVICEL-PH-105 (FMC Corporation, Marcus Hook, Pa.), and mixtures thereof. In some embodiment, a specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

In some embodiments, the pharmaceutical composition comprises one or more disintegrants. In certain embodiments, the disintegrant is selected from alginic acid, calcium alginate, carboxymethylcellulose calcium, carboxymethylcellulose sodium, cellulose, chitosan, colloidal silicon dioxide, corn starch, croscarmellose sodium, crospovidone, docusate sodium, glycine, guar gum, hydroxypropyl cellulose, magnesium aluminum silicate, methylcellulose, microcrystalline cellulose, pre-gelatinized starch, polacrilin potassium, povidone, silicates, sodium aliginate, sodium carbonate, and sodium starch glycolate.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

In some embodiments, the pharmaceutical composition comprises one or more surfactants. In some embodiments, the surfactant is selected from polyoxyethylene (20) sorbitan monolaurate (e.g., Tween-20), polyoxyethylene (20) sorbitan monooleate (e.g., Tween-80), sodium lauryl sulfate, and sodium dodecyl sulfate.

In some embodiments, the pharmaceutical composition comprises one or more pore formers. In some embodiments, the pore former is selected from hydroxypropylcellulose, hydroxypropylmethylcellulose, polyethyleneglycol, poloaxamer 188, povidone (e.g., Kollidon K25/K30), or sugar (e.g., glucose, mannose, fructose, and sucrose).

In some embodiments, the pharmaceutical composition comprises one or more glidants. In some embodiments, the glidant is selected from calcium phosphate, cellulose, colloidal silicon dioxide, fumed silica, magnesium oxide, magnesium silicate, magnesium stearate, magnesium trisilicate, and talc. Suitable glidants include, but are not limited to, colloidal silicon dioxide, CAB-O-SIL™ (Cabot Co. of Boston, MA), and asbestos-free talc.

In some embodiments, the pharmaceutical composition comprises one or more lubricants. In some embodiments, the lubricant is selected from calcium stearate, glycerin monosterate, glyceryl behenate, glyceryl palmitostearate, hydrogenated castor oil, hydrogenated vegetable oil, light mineral oil, myristic acid, poloxamer, polyethylene glycol, sodium benzoate, sodium chloride, sodium lauryl sulfate, sodium stearyl fumarate, solid polyethylene glycols, stearic acid, and talc.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof.

In some embodiments, the pharmaceutical composition comprises one or more film coating agents. In some embodiments, the film coating comprises a poly(vinyl alcohol) base. In some embodiments, the film coating includes a coloring agent or pigment. In some embodiments, the film coating is Opadry II® such as Opadry II® yellow.

Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Suitable flavoring agents include, but are not limited to, natural flavors extracted from plants, such as fruits, and synthetic blends of compounds which produce a pleasant taste sensation, such as peppermint and methyl salicylate.

Suitable sweetening agents include, but are not limited to, sucrose, lactose, mannitol, syrups, glycerin, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (Tween-20), polyoxyethylene sorbitan monooleate 80 (Tween-80), and triethanolamine oleate.

Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic acid, sodium benzoate, and alcohol.

Suitable wetting agents include, but are not limited to, propylene glycol monostearate, sorbitan monooleate, diethylene glycol monolaurate, and polyoxyethylene lauryl ether.

Suitable solvents include, but are not limited to, glycerin, sorbitol, ethyl alcohol, and syrup.

Suitable non-aqueous liquids utilized in emulsions include, but are not limited to, mineral oil and cottonseed oil.

Suitable organic acids include, but are not limited to, citric and tartaric acid.

Suitable sources of carbon dioxide include, but are not limited to, sodium bicarbonate and sodium carbonate.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

A tablet dosage form can be prepared from the active ingredient in powdered, crystalline, or granular forms, alone or in combination with one or more carriers or excipients described herein, including binders, disintegrants, controlled-release polymers, lubricants, diluents, and/or colorants.

A tablet of the present disclosure can be formulated for rapid, sustained, extended, or modified release.

In some embodiments, a unit dosage form comprises one or more pharmaceutically acceptable excipients selected from microcrystalline cellulose, lactose monohydrate (modified), croscarmellose sodium, hydroxypropyl cellulose, and magnesium stearate.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

The active compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops can be prepared. Additionally, transdermal patches can be used, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a composition, as described herein, can include a prodrug of Compound 1. The term “prodrug,” as used herein, means a compound that is convertible in vivo by metabolic means (e.g., by hydrolysis) to a compound. Various general forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, 9 9=., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrants include, without limitation, starch, methyl cellulose, agar, bentonite, xanthan gum starches, agar, alginic acid or its sodium salt, or effervescent mixtures, croscarmellose or its sodium salt, and the like. Diluents include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid: binding agents, for example starch, gelatin or acacia, and lubricating agents, for example magnesium stearate, stearic acid or talc. The tablets may be uncoated, or they may be coated by known techniques to delay disintegration and absorption in the gastrointestinal tract and thereby provide a sustained action over a longer period.

A therapeutically effective dose, of a compound described herein in an oral formulation, may vary from 0.15 mg/kg to 20 mg/kg patient body weight per day, more particularly 0.015 to 1.0 mg/kg, which can be administered in single or multiple doses per day. For oral administration, the drug can be delivered in the form of tablets or capsules containing 1 mg to 100 mg of the active ingredient specifically, 1 mg, 5 mg, 10 mg, 20 mg, 50 mg, or 100 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

For administration by inhalation, the compounds can be delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Penetration enhancers promote the penetration of drugs through the corneal barrier and change the integrity of the epithelial cell layer. Frequently used penetration enhancers in ocular formulations include cyclodextrin, dimethylsulphoxide (DMSO), ethylenediaminetetraacetic acid (EDTA), sodium glycocholate and related cholates, Tween 20 (a non-ionic polysorbate surfactant), Brij 35 (polyoxyethylene lauryl ether), saponins and bile salts. Generally, penetration enhancers such as EDTA and cholates transiently loosen the tight junctions between adjacent cells of the corneal epithelium. Thus, penetration enhancers, when applied topically to the eye, have been successfully applied to the delivery of protein and peptides through the corneal epithelium. In some embodiments, a formulation described herein includes a penetration enhancer such as polyoxyethylene-9-lauryl ether, sodium deoxycholate, sodium glycocholate, or sodium taurocholate.

Parenteral formulations comprising the compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as by injection, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

In some embodiments, the compound or pharmaceutically acceptable salt thereof is administered systemically. In some embodiments, the compound or pharmaceutically acceptable salt thereof is administered orally as part of a solid pharmaceutical composition. In some embodiments, the pharmaceutical composition is a liquid. In some embodiments, the pharmaceutical composition is administered as a liquid via nasogastric tube.

In some embodiments, the compound or pharmaceutically acceptable salt thereof is administered once, twice, thrice, or four times per day. In some embodiments, the compound or pharmaceutically acceptable salt thereof is administered twice per day. In some embodiments, the dose of the compound or pharmaceutically acceptable salt thereof is between about 1 mg BID (i.e., twice per day) to about 20 mg BID.

In some embodiments, the pharmaceutical composition is administered daily in one or more divided doses. In some embodiments, the composition is administered once per day (qua diem; QD). In some embodiments, the composition is administered twice per day (bis in die; BID). In some embodiments, the composition is administered thrice per day (ter in die; TID). In some embodiments, the composition is administered four times per day (quater in die; QID). In some embodiments, the composition is administered every four (4) hours (quaque four hours; q4h).

In some embodiments, the solid form of Compound 1 is substantially amorphous or crystalline or is a mixture thereof. In some embodiments, the solid form is substantially free of impurities.

In certain embodiments, Compound 1 is a crystalline solid. In some embodiments, Compound 1 is a crystalline solid substantially free of amorphous Compound 1. As used herein, the term “substantially free of amorphous Compound 1” means that the compound contains no significant amount of amorphous Compound 1. In some embodiments, at least about 95% by weight of crystalline Compound 1 is present. In still other embodiments, at least about 99% by weight of crystalline Compound 1 is present.

The compound may be formulated as an SDD formulation. “SDD” as used herein, refers to a pharmaceutical formulation (e.g., of Compound 1 or a pharmaceutically acceptable salt thereof) which is a spray dried formulation. In some embodiments, the formulation comprises a compound of the disclosure (e.g., Compound 1 or a pharmaceutically acceptable salt thereof) and hypromellose acetate succinate (HPMCAS). In one embodiment, the HMPCAS is HPMCAS-M, wherein the “M” indicates (acetyl content 7.0% to 11.0%, succinoyl content 10% to 14%). The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See European Patent Applications Nos. 0 901 786, 1 027 886, 1 027 887, 1 027 888, and PCT Applications Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

“TPGS” or “Vitamin E TPGS” as a descriptor for a pharmaceutical formulation for a compound of the disclosure, as used herein, refers to a pharmaceutical formulation (e.g., of Compound 1 or a pharmaceutically acceptable salt thereof) which includes the components of (a) the active compound (e.g., Compound 1 or a pharmaceutically acceptable salt thereof); (b) one or more diluents (e.g., microcrystalline cellulose); (c) one or more solubilizers (e.g., D-α-tocopherol polyethylene glycol succinate [Vitamin E TPGS]); and (d) one or more binders (e.g., povidone). The formulation may be prepared using granulation processes (e.g., wet granuation). “Granulation,” as used herein, refers to a process to produce larger or smaller granules or particles of a substance or mixture of substances. The process also may remove fine granules and improve flowability within the formulation. Both wet granulation and/or dry granulation may be employed. Dry granulation is achieved using only a combination of granules without the need for any liquid thereon. Slugging uses a tablet press to form large tablets that vary in weight due to the poor flowability of the formulation. The slugs created are then put through a granulator to be broken down into granules and then compressed once again for a final granulated product.

All publications, patents, patent applications and other documents cited in this application are hereby incorporated by reference in their entireties for all purposes to the same extent as if each individual publication, patent, patent application or other document were individually indicated to be incorporated by reference for all purposes.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds described herein, the following general methods, and other methods known to one of ordinary skill in the art, can be applied to all compounds and subclasses and species of each of these compounds, as described herein.

### Example 1: Polymorph Screen

The as-received material that were used to begin the polymorph screen are summarized in Table 1.

TABLE 1
Materials to begin polymorph screen

| Compound | Description |
|---|---|
| 1 | Form A + minor |
| | Material B |

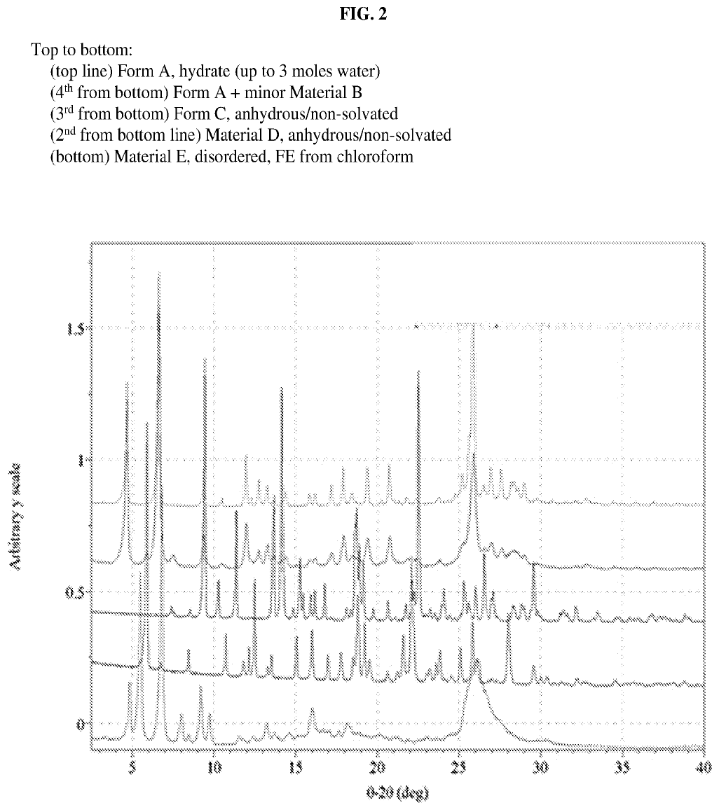

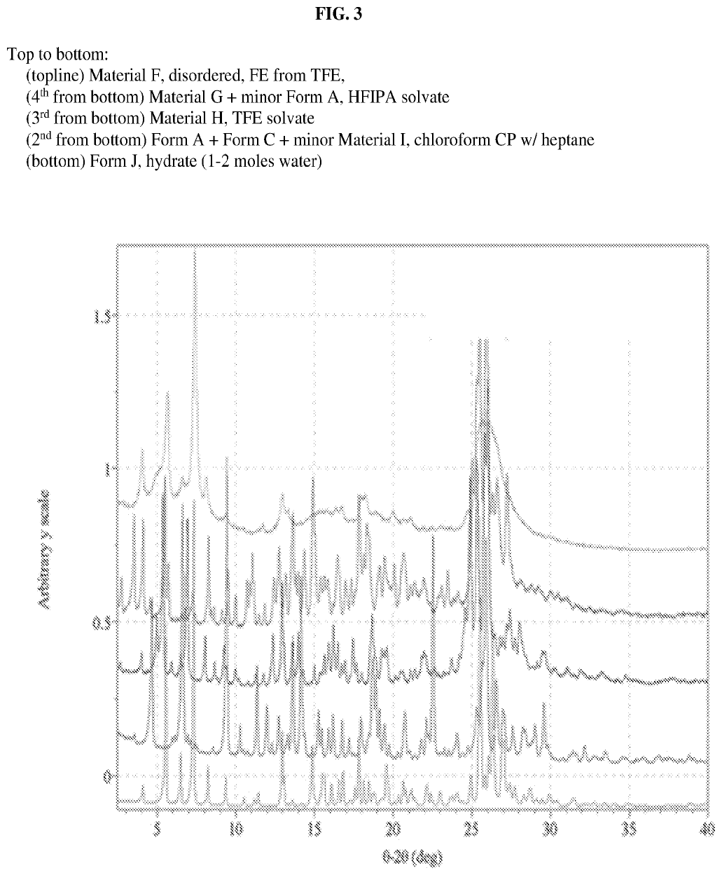

Eight named materials were discovered during the screen, designated as Materials/Forms C through J (FIGS. 2 and 3). Key information about each material is summarized in Table 13 and described in detail supra.

Two of the forms discovered during the screen, Form C and Material D, were confirmed to be anhydrous/non-solvated. Form A and Form J are hydrated. All other named materials are solvated, disordered, or were observed only in mixtures.

Early in the study, slurries were set up for the stable form screen at RT (Table 5, infra). In these experiments, solids of the as-received mixture of Form A+minor Material B were combined with given solvent systems at a solid loading that allowed for undissolved solids to remain, and the mixtures were stirred at ambient conditions. After 2 days, color changes to green, blue, or gray hues were noted for the slurries, a possible indicator of light sensitivity of either the API or the impurity present in the as-received material. Two slurries were selected for isolation at that time to confirm the solid form and chemical composition, while the remaining slurries were covered with foil as a precaution. The slurry in THF, which was observed as a green suspension (darker than many of the other slurries), caused conversion to a new crystalline form, designated as Form C. The other isolated slurry, from acetone/water 80:20, produced Form A as a single phase. Proton NMR spectra for both materials were consistent with the chemical structure of Compound 1, indicating that the color change did not correspond with significant degradation of the API solids (Table 2).

TABLE 2
Characterization Data for Compound 1 Materials and Forms

| Material/Form | Technique¹ | Results |
|---|---|---|
| Form A + | XRPD | Form A + minor Material B; |
| minor | | pattern could not be indexed |
| Material B, | OM | fines and aggregates, B/E |
| as-received | ¹H NMR | consistent with chemical structure, |
| | | minor impurity at 1.2 ppm, no |
| | | organic solvent detected |
| | DSC | small endo 108° C., overlapping exo 124° C., sharp |
| | | endo 244° C. (onset 242° C., 88 J/g) |
| | TGA | 6.3% weight loss 38-141° C. (eq. to 1.7 moles water) |
| | DVS | 0.00% weight change at 5% RH |
| | | 4.58% weight gain 5-95% RH (eq. to 1.2 moles) |
| | | 3.89% weight loss 95-5% RH |
| | post-DVS | Form A + minor Material B |
| | XRPD | |
| | KF | 5.88% water (eq. to 1.6 moles) |
| Form A | XRPD | Form A; |
| | | pattern successfully index, unit cell volume could |
| | | accommodate up to 3 moles water |
| | ¹H NMR | consistent with chemical structure, minor impurity at |
| | | 1.2 ppm, residual acetone present |
| | DSC | endo ~0° C., broad endos 91° C. and 103° C., sharp |
| | | endo 245° C. (onset 244° C., 72 J/g) |
| | TGA | 25.8% weight loss 37-136° C. |
| | KF² | 11.87% water (eq. to 3.4 moles) |
| Form A | XRPD | Form A |
| (from ~90% | KF | 7.29% water (eq. to 2.0 moles) |
| RH stress) | | |
| Form C | XRPD | Form C |
| | SCXRD | consistent with anhydrous/non-solvated Compound 1 |
| | | (density 1.324 g/cm³) |

| Material/Form | Technique³ | Results |
|---|---|---|
| Form C | XRPD | Form C; |
| (continued) | | pattern successfully indexed, unit cell volume |
| | | consistent with anhydrous/non-solvated Compound 1 |
| | ¹H NMR | consistent with chemical structure, no |
| | | organic solvent detected |
| | DSC | sharp endo 245° C. (onset 245° C., 100 J/g) |
| | TGA | 0.4% weight loss 39-248° C. |
| | DVS | 0.00% weight change at 5% RH |
| | | 1.75% weight gain 5-95% RH (eq. to ~0.5 mole) |
| | | 1.80% weight loss 95-5% RH |
| | post-DVS | Form C |
| | XRPD | |
| Material D | XRPD | Material D; |
| | | pattern successfully indexed, unit cell volume |
| | | consistent with anhydrous/non-solvated Compound 1 |
| | | (density 1.390 g/cm³) |
| Material E | XRPD | Material E, disordered |
| | ¹H NMR | consistent with chemical structure, no organic solvent |
| | | detected, minor impurities at low ppm |
| Material F | XRPD | Material F, disordered |
| | ¹H NMR | consistent with chemical structure, negligible TFE |
| | | present, minor impurities at low ppm |
| Material G + | XRPD | Material G + minor Form A; |
| minor | | pattern could not be indexed |
| Form A | ¹H NMR | consistent with chemical structure, 1.9 moles HFIPA |
| | | present, minor impurities at low ppm |
| | DSC | broad endos 83° C., 94° C., 138° C., overlapping endos |
| | | 241° C. and 245° C. (onset 230° C., total 46 J/g) |
| | TGA | 49.3% weight loss 39-159° C. |
| Material H | XRPD | Material H; |
| | | pattern could not be indexed |
| | ¹H NMR | consistent with chemical structure, 0.5 mole TFE |
| | | present, minor impurities at low ppm |
| | DSC | broad endos ~0° C. and 117° C., sharp endo 246° C. |
| | | (onset 244° C., 61 J/g) |
| | TGA | 32.3% weight loss 38-136° C. (eq. to 2.2 mole TFE) |
| Material/Form | Technique⁴ | Results |
| Form J | XRPD | Form J; |
| | | pattern could not be indexed |
| | ¹H NMR | consistent with chemical structure, 0.1 mole MeOH |
| | | present, minor impurities at low ppm |
| | DSC | broad endo 46° C., endo 135° C. with overlapping exo |
| | | 146° C., sharp endo 245° C. (onset 243° C., 99 J/g) |
| | TGA | insufficient sample |
| | XRPD | Form J; |
| | | pattern successfully indexed, unit cell volume could |
| | | accommodate up to 2 moles water |
| | ¹H NMR | consistent with chemical structure, 0.16 mole MeOH |
| | | present, minor impurities at low ppm |
| | DSC | broad enod 93° C., endo 135° C. with overlapping exo |
| | | 149° C., small endo 240° C., sharp endo 245° C. (onset |
| | | 244° C., 92/g) |
| | TGA | 5.8% weight loss 40-143° C. (eq to ~1.26 moles |
| | | water + 0.16 mole MeOH) |
| | KF | 5.064% (or 1.4 moles) water |
| amorphous | cycling | Step 1: −25 to 270° C. - sharp endo 245° C. (onset |
| attempt | DSC | 245° C., 67 J/g) |
| | | Step 2: cool to −25° C. - no observations |
| | | Step 3: −25 to 350° C. - T_{g} 110° C. (0.2 J/g*K), exo |
| | | 186° C., sharp endo 239° C. (onset 238° C., |
| | | 65 J/g) |

¹Temperatures (° C.) reported for DSC data are transition maxima unless otherwise stated.
²KF analysis was done ~1 month after TGA analysis.
³Temperatures (° C.) reported for DSC data are transition maxima unless otherwise stated.
⁴Temperatures (° C.) reported for DSC data are transition maxima unless otherwise stated.

Based on these results, the remaining RT slurries were allowed to stir for ˜2 weeks (Table 5). Additional slurries were set up at elevated temperatures (˜60° C. and ˜80° C.) and sub-ambient temperatures (2 to 8° C.) as well. Elevated-temperature slurries were stirred for much shorter durations in an effort to avoid any decomposition that may occur upon prolonged heating. No significant color changes were noted for those slurries, likely because they were shielded from light for the entire duration. All slurries conducted in organic solvents at all temperatures caused full conversion to Form C, indicating it is likely the most stable anhydrous/non-solvated form between 2° C. and 80° C. Slurries in aqueous/organic mixtures at or above water activity (aw) 0.82 resulted in Form A (hydrate) at RT and 2 to 8° C., while those at or below aw 0.69 caused full or partial conversion to Form C, suggesting the critical water activity between those forms lies between 0.69 and 0.82 at RT.

Polymorph screening using more kinetically-driven techniques produced a variety of new materials (Table 6, infra). Specific conditions that resulted in a given form are discussed in and tabulated below. Overall, the results indicate a propensity of Compound 1 to exist in a variety of solid forms, particularly solvates or hydrates.

Attempts to prepare amorphous material by melt/quench or rotary evaporation from chloroform were unsuccessful (Table 7).

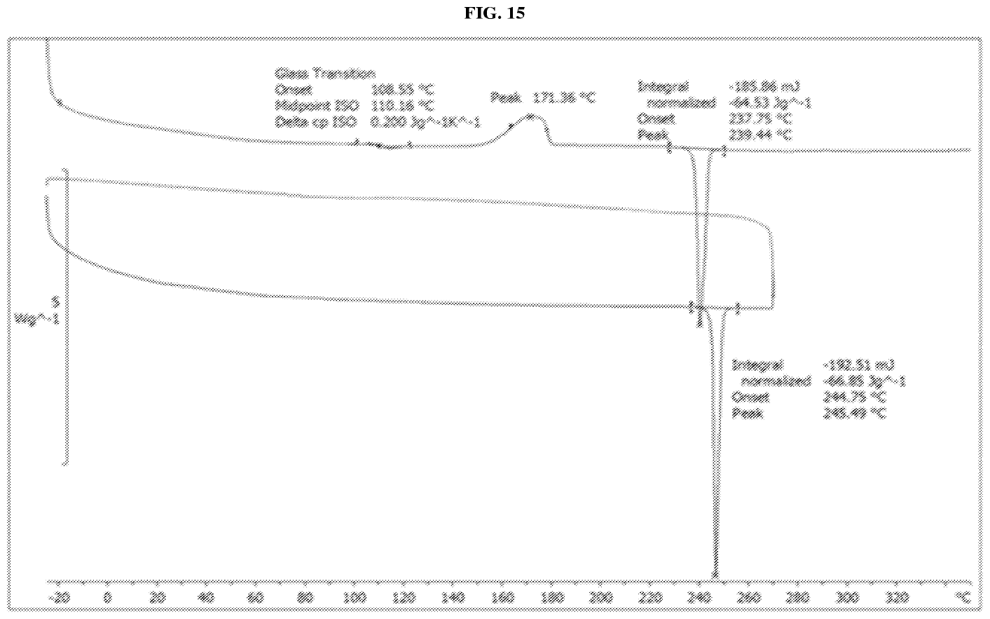

Therefore, amorphous material was not made in sufficient quantity for use as an alternative starting material for the polymorph screen. A cycling DSC experiment, though, was successful in melting and quenching solids without crystallization, allowing for observation of a glass transition event at 110° C., discussed in more detail infra (Table 2). This experiment also enabled the discovery of a new metastable anhydrous/non-solvated form, Material D, which crystallized upon heating the amorphous material above 160° C. (FIG. 15).

Select materials were stressed at ˜90% RH, slurried in relevant solvent systems, heated for desolvation, reproduced for further study, or competitively slurried at conditions of interest (Tables 8 to 12). These results are discussed in detail infra.

### Description of Named Materials and Known Forms of Compound 1:

Form A: Form A is a hydrate (up to 3 moles of water) of Compound 1 that resulted from numerous form screen experiments, particularly those at water activity conditions at or above 0.82. Three samples containing Form A were utilized for various characterization techniques: as-received Form A+minor Material B, Form A from a slurry in acetone/water 80:20, and Form A stressed at ˜90% RH (Table 2).

Characterization was by XRPD, OM, proton NMR, DSC, TGA, DVS, and KF (Table 2). The material was observed to consist of birefringent fines and aggregates by optical microscopy.

The XRPD pattern for lot 5 is shown in FIG. 4 (top). The pattern could not be indexed and was determined to consist of a mixture of primarily Form A with a minor Material B component upon the observation of Form A as a single phase from a slurry experiment. A comparison of the XRPD patterns, shown in FIG. 4, illustrates the additional peaks attributed to Material B with blue asterisks.

The XRPD pattern for Form A as a single phase, obtained by slurrying the as-received mixture in acetone/water 80:20 (aw 0.82) for 2 days, was successfully indexed (FIG. 5). The unit cell volume obtained from the indexing solution could accommodate Compound 1 with up to 3 moles of water, based on molecular size.

Proton NMR spectra were obtained for the as-received mixture and Form A as a single phase in deuterated DMSO. Both spectra were consistent with the chemical structure of Compound 1 with a minor unknown impurity exhibited at 1.2 ppm (likely not attributed to Material B since it was observed in both spectra). Minor residual acetone was present in the Form A spectrum.

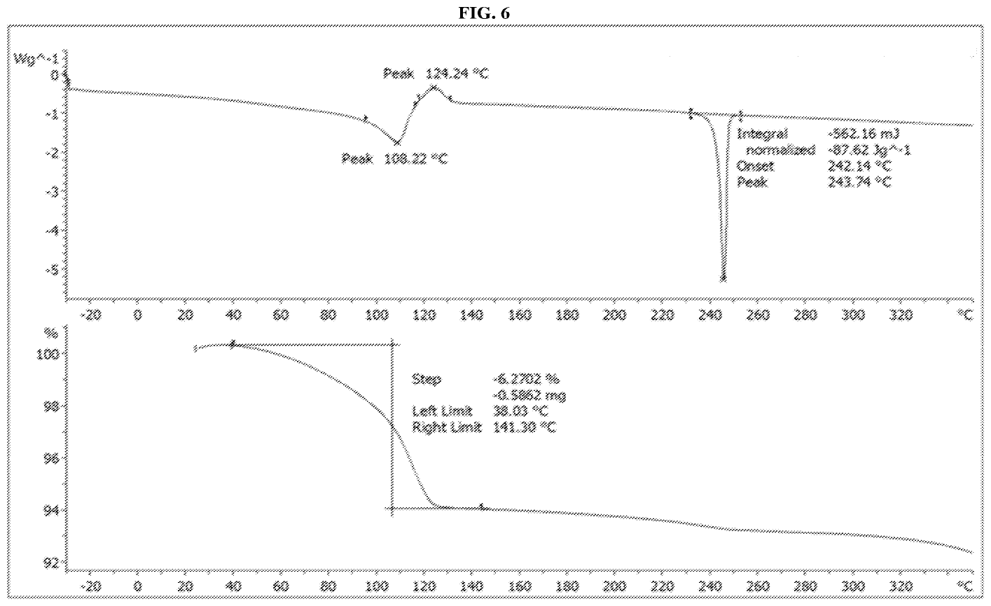

DSC and TGA thermograms for the as-received mixture and Form A as a single phase are shown in FIGS. 6 and 7, respectively. Both sets of data indicate a large weight loss step by TGA corresponding with broad endotherms by DSC up to ˜140° C., consistent with dehydration. The magnitude of the weight loss step exhibits a large variation between the samples, likely due to the presence of residual solvent in the generated Form A material. The small melting endotherm at 0° C. in the DSC thermogram for Form A confirms the presence of residual (un-bound) water in the sample. The 6.3% weight loss observed for the Form A+minor Material B mixture is equivalent to 1.7 moles of water, assuming water is the only volatile. An exotherm is evident immediately following the dehydration endotherm for the Form A+minor Material B mixture, likely indicating crystallization to a dehydrated form. A similar crystallization event also likely occurred for the Form A sample, but the exothermic component is likely buried by the larger endothermic event corresponding with the loss of a larger amount of water. The dehydrated crystalline materials in both thermograms exhibit a melting endotherm at 242 to 244° C. (onset).

Based on the thermal data, a sample of Form A was heated to 150° C. by TGA for dehydration, and the solids were recovered and analyzed by XRPD (Table 10). Full conversion to Form C (anhydrous/non-solvated stable form) was observed.

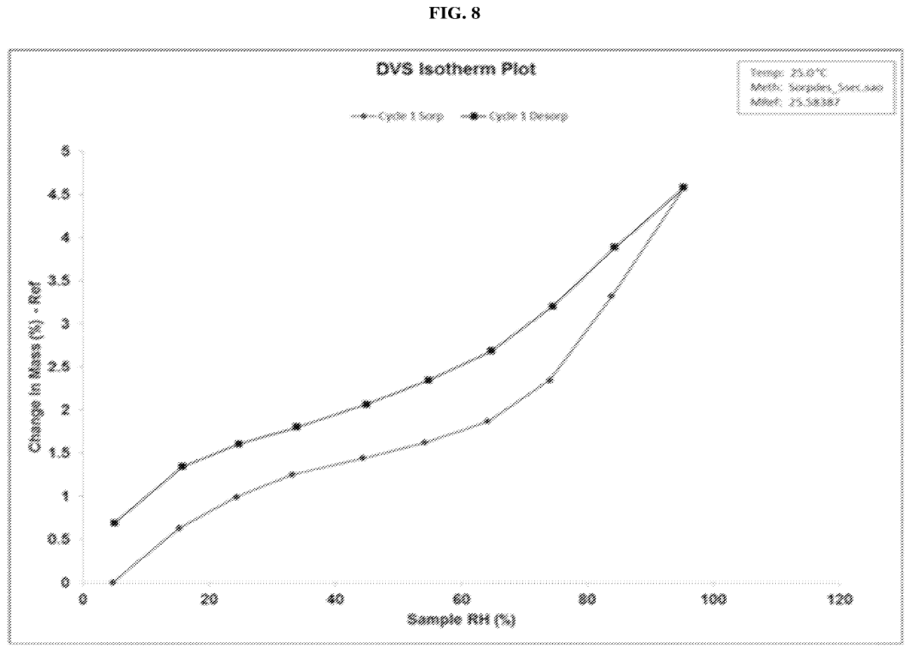

A DVS isotherm for the as-received mixture is shown in FIG. 8. The material exhibited 4.6 wt % water uptake between 5% and 95% RH (equivalent to 1.2 moles of water), indicating significant hygroscopicity. Nearly all of the sorbed water was lost upon desorption between 95% and 5% RH, retaining less than 0.7 wt %. XRPD of the post-DVS solids was consistent with a mixture of Form A with minor Material B.

Three samples containing Form A were analyzed by Karl Fisher for comparison of water content (Table 2). The as-received mixture of Form A+minor Material B contained 1.6 moles of water. The sample of Form A from the slurry experiment (previously found to contain significant residual solvent by DSC and TGA but analyzed approximately 1 month later by KF) contained 3.4 moles of water. A sample of Form A that had been equilibrated at ˜90% RH for 6 days (still consisting of Form A by XRPD, Table 8 infra)) contained 2.0 moles of water. The latter value is likely the most representative of the true, bound water content, and indicates a di-hydrate at ˜90% RH. As the DVS isotherm shows, the water content is variable and depends upon the ambient RH. Form A would likely contain no more than 3 moles of water, based upon the unit cell volume, as well as the sum of the weight loss by TGA (1.7 moles) and the sorption by DVS (1.2 moles).

The critical water activity between Form A and Form C (anhydrous/non-solvated) likely falls between 0.69 and 0.82 at RT and is discussed in more detail in infra.

Material B: Material B is a minor crystalline impurity of unknown composition observed in as-received lot 5 as a mixture with Form A. The material was not observed in the XRPD patterns for any of the stable form or polymorph screen experiments. Since it was not produced as a single phase, the material could not be further characterized. The XRPD overlay in FIG. 4 illustrates the peaks attributed to Material B. An insoluble impurity was observed while obtaining solubility estimations using as-received lot and may correspond with Material B, although additional testing would be needed to confirm this (Table 4, below).

Form C: Form C consists of anhydrous/non-solvated Compound 1 and is the likely stable non-solvated form between 2° C. and 80° C. Form C resulted from all stable form screen slurries in organic solvent systems between 2° C. and 80° C., slurries in aqueous mixtures at or below aw 0.69 at RT, and numerous polymorph screen and desolvation/dehydration experiments (Tables 5, 6, 7, and 10). Select detailed slurry procedures to convert Form A+minor Material B to Form C are disclosed therein. A single crystal structure for Form C was obtained, and the form was additionally characterized by XRPD indexing, proton NMR, DSC, TGA, and DVS (Table 2).

A single crystal of Form C was obtained from a slurry experiment in MEK at ˜60° C. (Table 5). The crystal system is monoclinic, and the space group is P21. The cell parameters and calculated volume are: a=12.46390(16) Å, b=15.55789(14) Å, c=12.69650(15) Å, α=90°, β=110.2065(14)°, γ=90°, V=2310.47(5) Å3. The molecular weight is 460.50 g mol-1 with Z=4, resulting in a calculated density of 1.324 g cm⁻³. Further details of the crystal data and crystallographic data collection parameters are summarized in Table 3.

TABLE 3
Crystal Data and Data Collection
Parameters for Compound 1 Form C.

| | |
|---|---|
| Empirical formula | C₂₃H₂₄N₈O₃ |
| Formula weight (g mol⁻¹) | 460.50 |
| Temperature (K) | 299.88(10) |
| Wavelength (Å) | 1.54184 |
| Crystal system | monoclinic |
| Space group | P2₁ |
| Unit cell parameters | |
| a = 12.46390(16) Å | α = 90° |
| b = 15.55789(14) Å | β = 110.2065(14)° |
| c = 12.69650(15) Å | γ = 90° |
| Unit cell volume (Å³) | 2310.47(5) |
| Cell formula units, Z | 4 |
| Calculated density (g cm⁻³) | 1.324 |
| Absorption coefficient (mm⁻¹) | 0.758 |
| F(000) | 968 |
| Crystal size (mm³) | 0.6 × 0.26 × 0.07 |
| Reflections used for | 16314 |
| cell measurement | |
| θ range for cell measurement | 3.6920°-77.3870° |
| Total reflections collected | 25050 |
| Index ranges | −15 ≤ h ≤ 14; −19 ≤ |
| | k ≤ 19; −14 ≤ l ≤ 16 |
| θ range for data collection | θₘᵢₙ = 3.710°, θₘₐₓ = 77.617° |
| Completeness to θₘₐₓ | 98.7% |
| Completeness to θ_{full} = 67.684° | 100% |
| Absorption correction | multi-scan |
| Transmission coefficient range | 0.863-1.000 |
| Refinement method | full matrix least-squares on F² |
| Independent reflections | 9477 [Rᵢₙₜ = 0.0237, R_{σ} = 0.0254] |
| Reflections [I > 2σ(I)] | 8578 |
| Reflections/restraints/parameters | 9477/1/642 |
| Goodness-of-fit on F² | S = 1.08 |
| Final residuals [I > 2σ(I)] | R = 0.0445, R_{w} = 0.1238 |
| Final residuals [all reflections] | R = 0.0484, R_{w} = 0.1277 |
| Largest diff. peak and hole (e Å⁻³) | 0.505, −0.325 |
| Max/mean shift/standard uncertainty | 0.000/0.000 |
| Absolute structure determination | Flack parameter: 0.11(10) |

TABLE 4
Approximate Solubility of Compound 1 in Various Solvents at
Ambient Temperature using substance Form A + minor Material B
Table 4: Approximate Solubility of Compound 1 in Various
Solvents at Ambient Temperature

| | Solubility | |
|---|---|---|
| Solvent | (mg/mL) | Observations |
| acetone | ~1 | clear with few floating particles |
| ACN | <1 | solids present |
| chloroform | 31 | T₀: clear solution |
| | | 3 d at RT: clear light green soln. |
| p-dioxane | 2 | clear with few floating particles |
| EtOAc | <1 | solids present |
| EtOH | <1 | solids present |
| HFIPA | ~>100 | T₀: clear with few floating particles |
| | | 3 d at RT: clear yellow soln. w/ |
| | | few floating particles |
| MeOH | ~2 | clear with few floating particles |
| | | (Note: adding additional MeOH to 1 |
| | | mg/mL achieved clear solution) |
| MTBE | <1 | solids present |
| TFE | ~>78 | T₀: clear with few floating particles |
| | | 3 d at RT: clear light green soln. w/ |
| | | few floating particles |
| THF | ~3 | clear with few floating particles |
| toluene | <1 | solids present |
| water | <1 | solids present |
| acetone/water 50:50 | <1 | solids present |
| MeOH/water 75:25 | ~2 | clear with few floating particles |
| THF/water 90:10 | 10 | clear soln. |

Solubilities are calculated based on the total solvent used to give a solution; actual solubilities may be greater because of the volume of the solvent portions used or a slow rate of dissolution. Values are rounded to the nearest whole number. If dissolution did not occur as determined by visual assessment, the value is reported as “<”. If dissolution occurred as determined by the visual assessment after the addition of the first aliquot, the value is reported as “>”.

TABLE 5
Stable Form Screen Slurries of Compound 1

| | | | XRPD |
|---|---|---|---|
| Solvent | Conditions | Observations | Result |
| acetone | RT, 14 d | 1) light green color noted | C |
| | | after 2 d | (analyzed |
| | | 2) opaque blue-green | damp) |
| | | suspension, fines; limited | |
| | | aciculars/blades, B | |
| ACN | RT, 14 d | 1) light green color noted | C |
| | | after 2 d | (analyzed |
| | | 2) opaque pale yellow- | damp) |
| | | green suspension; limited | |
| | | aciculars/blades, fines, B | |
| | ~60° C., 3 d | opaque off-white | C |
| | | suspension, few light | |
| | | green and brown solids | |
| | | on upper walls; fines and | |
| | | few thin needles, B/E | |
| p-dioxane | RT, 14 d | 1) light green color noted | C |
| | | after 2 d | (analyzed |
| | | 2) opaque pale yellow- | damp) |
| | | green suspension; | |
| | | fines, B | |
| EtOAc | ~60° C., 3 d | opaque off-white | C |
| | | suspension; fines, B/E | |
| EtOH | RT, 14 d | 1) light green color noted | C |
| | | after 2 d | (analyzed |
| | | 2) opaque pale blue- | damp) |
| | | green suspension; | |
| | | fines, B | |
| | ~60° C., 3 d | opaque off-white | C |
| | | suspension, few amber- | |
| | | colored solids on upper | |
| | | walls; fines and few thin | |
| | | needles, B/E | |
| HFIPA/EtOAc | RT, 14 d | 1) darker green color | C |
| 5:95 | | noted after 2 d | (analyzed |
| | | 2) opaque blue-green | damp) |
| | | suspension; fines, B | |
| IPA | RT, 14 d | 1) light gray color noted | C |
| | | after 2 d | (analyzed |
| | | 2) opaque pale green- | damp) |
| | | yellow suspension; fines, | |
| | | B | |
| MEK | ~60° C., 3 d | opaque off-white | C |
| | | suspension, few amber | |
| | | and light green solids on | |
| | | upper walls; fines and | |
| | | few rectangular plates on | |
| | | walls, B/E (likely singles) | |
| MeOH | RT, 14 d | 1) light green color noted | C |
| | | after 2 d | (analyzed |
| | | 2) opaque pale yellow- | damp) |
| | | green suspension; | |
| | | fines, B | |
| | 2-8° C., 20 d | opaque off-white | C |
| | | suspension; fines, B/E | |
| | ~60-61° C., 3 d | opaque off-white | C |
| | | suspension; fines, B/E | |
| MIBK | ~80° C., 1 d | opaque light yellow | C |
| | | suspension; fines and few | |
| | | needles on walls, B/E | |
| THF | 1) RT, 2 d | 1) opaque darker green | C |
| | 2) filter | suspension; fines, B/E | |
| | | 2) slightly green solids | |
| | ~60-61° C., 3 d | cloudy light yellow | C |
| | | suspension; fines, B/E | |
| acetone/water | 1) RT, 2 d | 1) opaque blue-green | A |
| 80:20 (a_{w} 0.82) | 2) filter | suspension; fines, B/E | |
| | | 2) slightly green solids | |
| | 2-8° C., 20 d | opaque slightly green | A |
| | | suspension; fines, B/E | |
| acetone/water | RT, 10 d | opaque off-white | C + |
| 90:10 (a_{w} 0.69) | | suspension; fines, B/E | minor A |
| MeOH/water | RT, 14 d | 1) light green color | C |
| 75:25 (a_{w} 0.54) | | noted after 2 d | (analyzed |
| | | 2) opaque green-yellow | damp) |
| | | suspension; fines, B | |
| THF/water | RT, 14 d | 1) blue-gray color | |
| 90:10 (a_{w} ~1) | | noted after 2 d | A |
| | | 2) opaque red-purple | (analyzed |
| | | suspension, fines, B | damp) |
| | 2-8° C., 20 d | opaque off-white | A |
| | | suspension; fines, B/E | |

TABLE 6
polymorph Screen of Compound 1

| Solvent | Conditions | Observations | XRPD Result |
|---|---|---|---|
| chloroform | FE | light greenish-gray | E, |
| | | solids; small needles, | disordered |
| | | fines, and aggregates, | |
| | | B/E | |
| | CP w/MTBE | ppt. w/2^{nd} aliquot, | C + |
| | | cloudy off-white | minor D |
| | | suspension; fines, | |
| | | B/E | |
| | 1) CP w/heptane | 1) cloudy w/1^{st} | A + C + |
| | 2) stir at RT, 7 d | aliquot, translucent | minor I |
| | | aggregates, B/E | |
| | | at edges | |
| | | 2) opaque white | |
| | | suspension; | |
| | | fines, B/E | |
| | VD w/diethyl | clear liquid and off- | C, low |
| | ether, RT, 1 d | white solids; | signal |
| | | spherulites of fine | (analyzed |
| | | needles, B/E | damp) |
| | VD w/toluene, | damp light | A + |
| | RT, 3 d | yellow/green solids; | minor C |
| | | needles and | (analyzed |
| | | agglomerates, B/E | damp) |
| HFIPA | FE | off-white film; | disordered |
| | | fines, B/E | with several |
| | | | broad features |
| | 1) CP w/water | 1) ppt. w/2^{nd} aliquot, | G + |
| | 2) ref, 16 d | cloudy liquid, yellow | minor A |
| | | oil on bottom | |
| | | 2) clear liquid and | |
| | | off-white solids; | |
| | | aggregates of fine | |
| | | needles, B/E | |
| MeOH | CC, ~60-61° C. | opaque white | J, PO |
| | to frz, stand | suspension; fine | |
| | in frz 4 d | needles, B/E | |
| TFE | FE | off-white solids/film; | F, |
| | | dendritic formations | disordered |
| | | and fines, B/E | |
| | 1) add EtOAc | 1) clear soln. | C |
| | (TFE/EtOAc 1:6) | 2) opaque white | |
| | 2) stir in frz, | suspension; | |
| | 4 d | fines, B/E | |
| TFE | 1) CP w/diethyl | 1) ppt w/2^{nd} aliquot, | C |
| (continued) | ether | opaque off-white | |
| | 2) stir at RT, | suspension; | |
| | 1 d | aggregates, some B/E | |
| | | 2) opaque off-white | |
| | | suspension; tiny | |
| | | needles, B/E | |
| | VD w/diethyl | clear liquid and off- | C |
| | ether, RT, 1 d | white solids; | |
| | | aggregates of fine | |
| | | needles, B/E | |
| | VD w/water, | off-white solids/film | H |
| | RT, 3 d | on walls, no liquid; | |
| | | dendritic formations | |
| | | and tiny needles, B/E | |
| THF | 1) CC, ~60-61° C. | 1) clear soln. | C |
| | to frz, stand | 2) clear liquid and | |
| | in frz 4 d | light yellow solids | |
| | 2) VD w/diethyl | coating bottom; | |
| | ether, RT, 3 d | spherulites of needles | |
| | | and large rectangular | |
| | | plates, B/E (indexing | |
| | | of a single crystal | |
| | | indicated Form C; | |
| | | structure not | |
| | | collected) | |
| THF/water | SE | off-white solids; thin | A |
| 90:10 | | dendritic needles, | (analyzed |
| | | B/E (damp) | damp) |

TABLE 7
Attempts to Prepare Amorphous Compound 1

| | | XRPD |
|---|---|---|
| Conditions | Observations | Result |
| 1) melt on hot | 1) dark brown pool of liquid, | C |
| plate at ~280° C. | small amt. of powder on | |
| 2) quench to frz, ~1.5 h | walls (partial B/E) | |
| 3) store in frz | 2) cracked brown glass | |
| | (no B/E) on bottom, few | |
| | B/E fines on walls | |
| | 3) — | |
| 1) RE from chloroform at RT | 1) light yellow bubbly, | C + |
| 2) store in frz | glassy solids; no B/E | D + E |
| | 2) — | |

TABLE 8
Stressing Experiments for Select Compound 1 Forms

| Sample Source | Conditions | Observations | XRPD Result |
|---|---|---|---|
| Form A | ~90% RH, | free-flowing light | A |
| | RT, 6 d | green solids; | |
| | | aggregates, no B/E | |
| Form C | ~90% RH, | free-flowing off- | C |
| | RT, 11 d | white solids; fines | |
| | | and aggregates, B/E | |

TABLE 9
Slurry Experiments with Compound 1 Form C

| Sample Source | Conditions | Observations | XRPD Result |
|---|---|---|---|
| Form C, | slurry in water | opaque off-white | C |
| | (a_{w} 1), 1 d | suspension; | |
| | | fines, B/E | |
| Form C, | slurry in | opaque off-white | A |
| | THF/water 50:50 | suspension; tiny | |
| | (a_{w} ~1), 1 d | needles, B/E | |

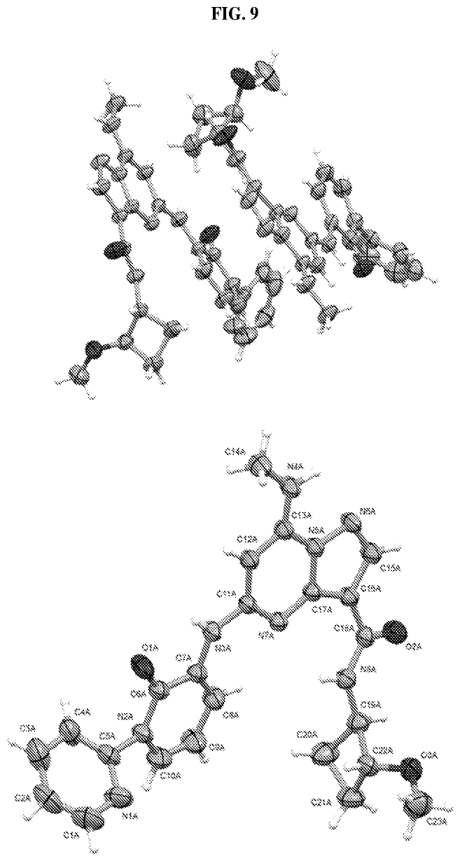

The quality of the structure obtained is high, as indicated by the fit residual, R, of 0.0445 (4.45%). R-factors in the range 2%-6% are quoted to be the most reliably determined structures. An atomic displacement ellipsoid drawing of Form C is shown in FIG. 9. The asymmetric unit shown in FIG. 9 contains two Compound 1 molecules. An XRPD pattern was calculated from the SCXRD data and is in good agreement with a pattern measured experimentally (FIG. 10).

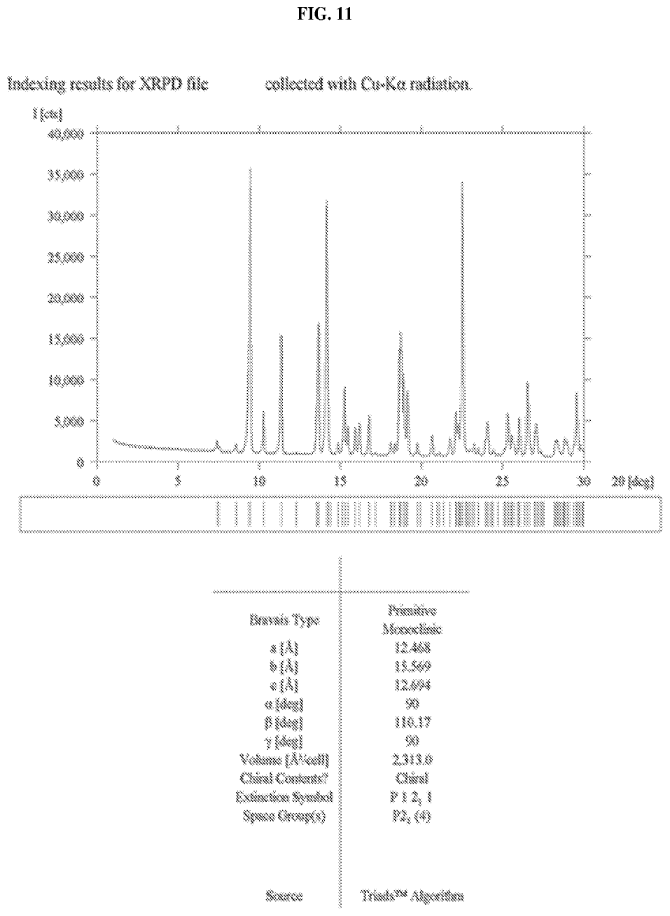

An XRPD pattern for Form C was successfully indexed (FIG. 11). The unit cell parameters are in good agreement with those obtained by SCXRD and are consistent with anhydrous/non-solvated Compound 1.

A proton NMR spectrum for Form C was consistent with the chemical structure of Compound 1 with no organic solvent detected (Table 2).

DSC and TGA thermograms are shown in FIG. 12. Negligible weight loss is observed through 300° C., consistent with an anhydrous/non-solvated material. A sharp endotherm at 245° C. (onset) likely corresponds to the melt.

A DVS isotherm for Form C is shown in FIG. 13 (Table 2). Limited hygroscopicity was observed, with the material picking up 1.75% water (equivalent to ˜0.5 mole) between 5% and 95% RH. All of this weight was lost on desorption with no hysteresis. XRPD of the post-DVS material was consistent with Form C.

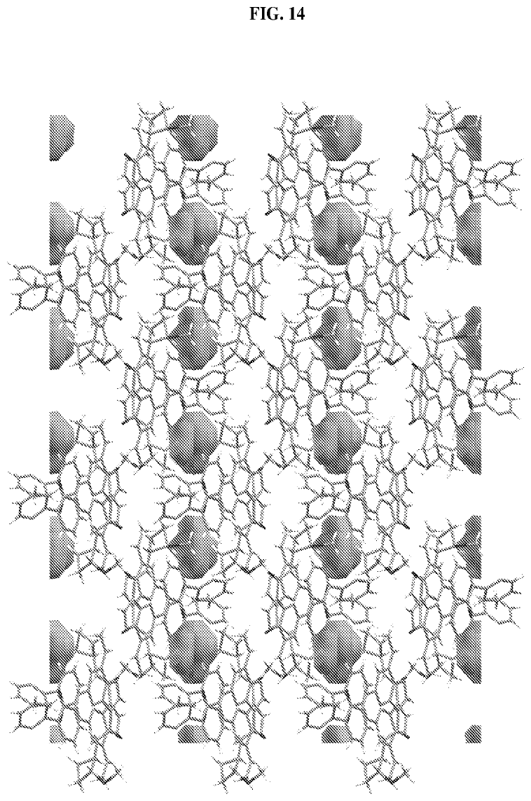

The single crystal structure of Form C indicated an anhydrous form that contains void spaces throughout the structure (illustrated as yellow spaces in FIG. 14). These void spaces are large enough to accommodate up to 0.5 mole of water, with the amount of sorbed water dependent upon the surrounding relative humidity. The lack of hysteresis in the DVS isotherm suggests that the material equilibrates quickly and that the water can enter/leave easily.

To further study the behavior at high RH, a sample of Form C was stressed at ˜90% RH for 11 days (Table 8). XRPD of the resulting solids was consistent with Form C, confirming that a form change did not occur upon sorption of water over that duration. However, knowing that a crystalline hydrate exists at water activity conditions at or above 0.82 (i.e. at or above 82% RH), Form C would be expected to convert to hydrated Form A upon longer exposure at water activity conditions above the critical aw.

Slurries of Form C at high water activity conditions and interconversion slurries containing Form C are discussed with respect to relative thermodynamic stability of forms in infra.

Material D: Material D consists of an anhydrous/non-solvated form of Compound 1 that is metastable at RT and 2 to 8° C. The material resulted from a cycling DSC experiment starting with Form C (FIG. 15, Table 2). The experiment was initially performed to target amorphous material for observation of a glass transition event. In the experiment, the solids were heated past the melt (observed as a sharp endotherm at onset 245° C. in the first leg), then cooled back to −25° C. in the second leg and reheated in the third leg. The final heating step showed a glass transition at 110° C., followed by a crystallization exotherm at 171° C. (presumably to Material D) and a likely melt at 238° C. (onset).

Based on this data, a sample of Form C was heated by TGA past the melt (to 270° C.), quenched to 40° C., and then reheated to 200° C. (Table 10).

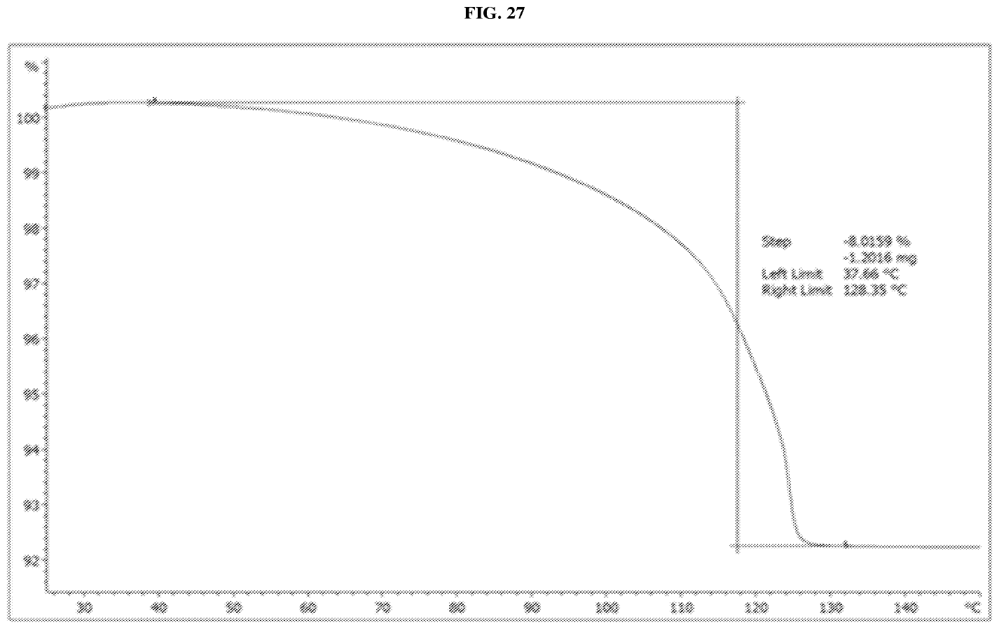

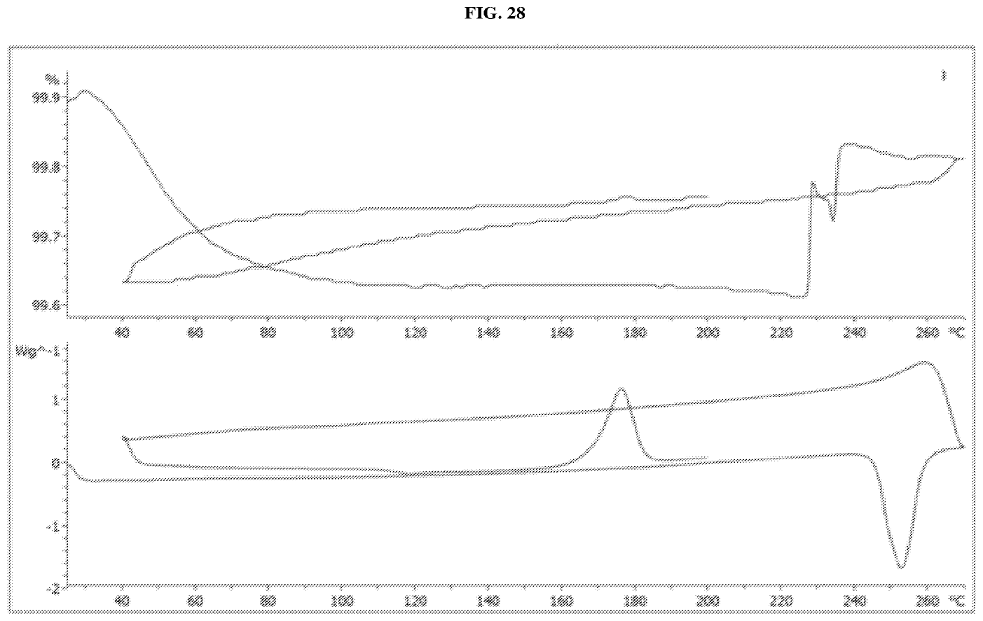

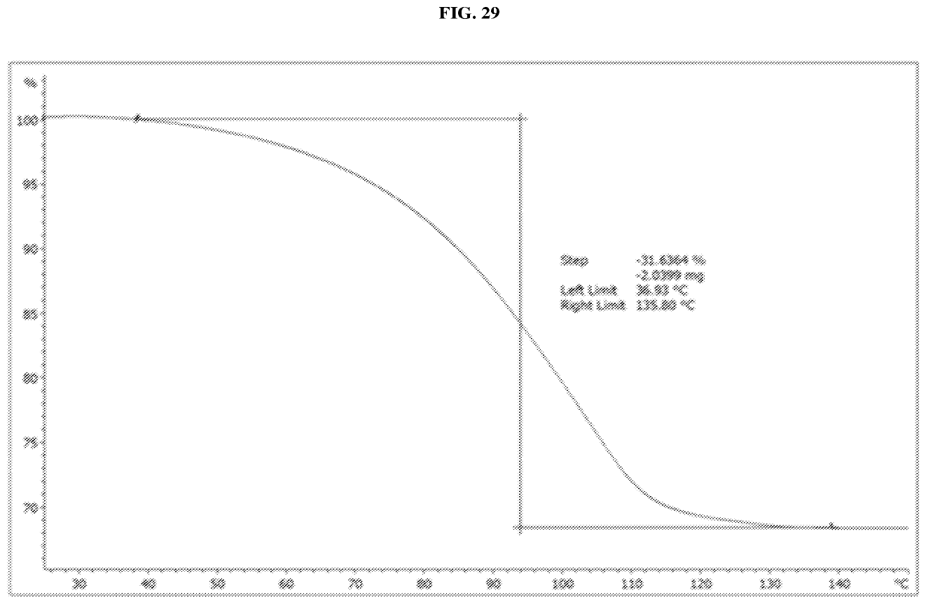

TABLE 10
Heating Experiments for Select Compound 1 forms.

| Sample Source | Conditions | Observations | XRPD Result |
|---|---|---|---|
| Form A, | heat to 150° C. (TGA) | — | C |
| | FIG. 27 | | |
| Form C, | heat to 270° C., cool to | — | D |
| | 40° C., heat to 200° C. | | |
| | (TGA) | | |
| | FIG. 28 | | |
| Material H, | heat to 150° C. (TGA) | — | C |
| | FIG. 29 | | |

The solids were recovered and were found to consist of Material D by XRPD (FIG. 16). This experiment was repeated to obtain additional material but was not characterized by XRPD to conserve solids (Table 11). To be noted, Material D was previously observed as a minor component in mixtures with Form C and Material E from polymorph screen experiments in chloroform (Tables 6 and 7, above).

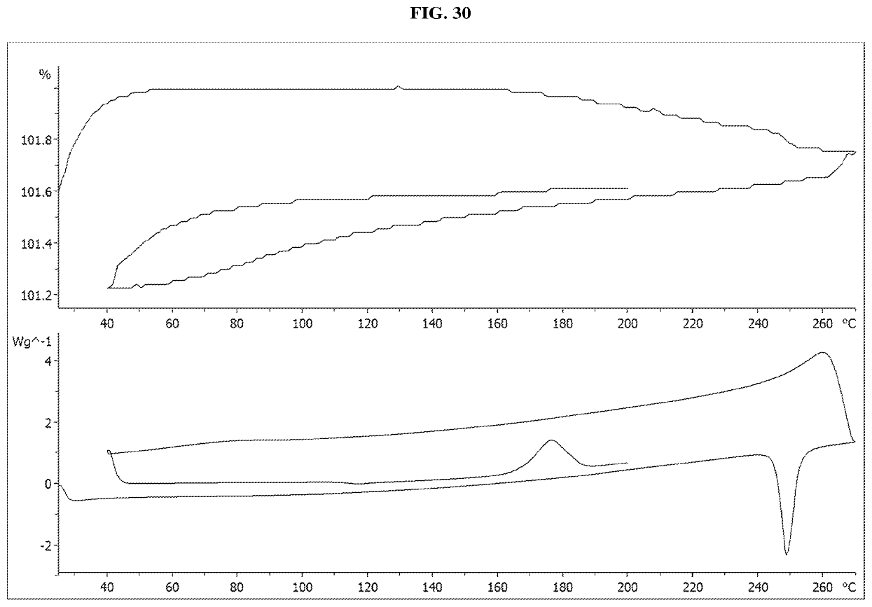

TABLE 11
Attempts to Reproduce Named Materials and Forms of Compound 1

| Intended | | | XRPD |
|---|---|---|---|
| Material/Form | Conditions | Observations | Result |
| Material D | heat Form C to 270° C., | TGA/DSC exhibits | insufficient |
| | cool to 40° C., heat to | likely recryst. | solids |
| | 200° C. (TGA/DSC) | event, presumably | |
| | FIG. 30 | to Material D | |
| Form J | 1) add MeOH to Form | 1) cloudy | J |
| | A + minor Material B | suspension | |
| | w/stirring at ~61° C. | 2) clear soln. | |
| | 2) hot filter | 3) seeds remained | |
| | 3) SC ~ 10 m (to | 4) clear soln. w/ | |
| | ~55° C.), add seeds | seeds on bottom | |
| | of Form J | 5) opaque white | |
| | 4) continue SC to RT | suspension; | |
| | 5) frz, 1 d | aggregates of very | |
| | 6) filter while cool | fine needles, B/E | |
| | 7) scrape and break | 6) damp, off-white | |
| | apart with spatula | solids | |
| | few min | 7) free-flowing | |
| | | powder | |

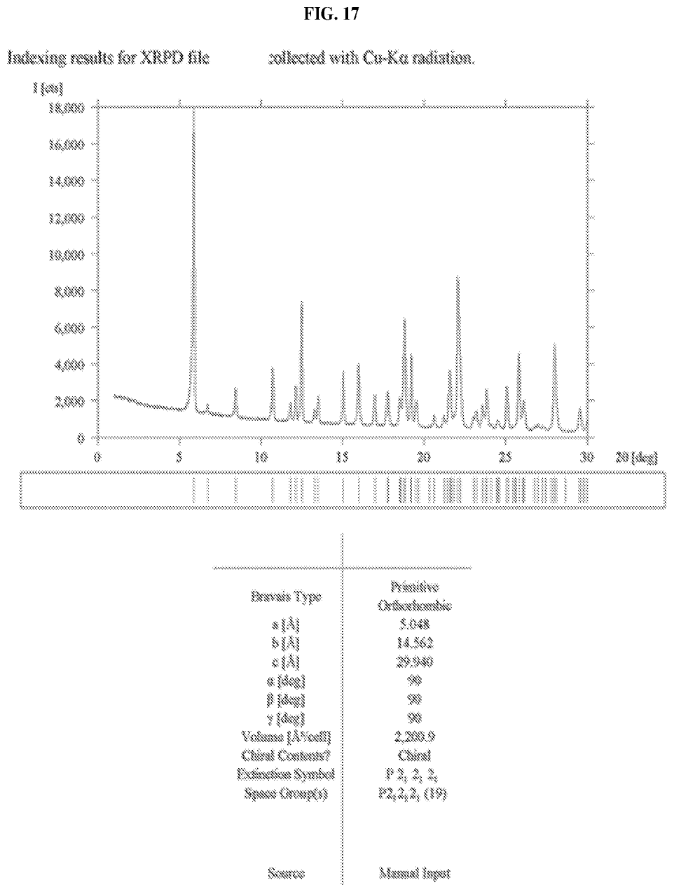

The XRPD pattern for Material D was successfully indexed (FIG. 17). The unit cell volume is consistent with anhydrous/non-solvated Compound 1 with higher density than Form C (1.390 g/cm³ versus 1.324 g/cm³). Discussion of how this information pertains to the relative thermodynamic stability of these forms is provided in infra below.

Material limitations prevented any additional characterization of Material D. By the technique described above, the material was only made in small quantities, and all solids were utilized for interconversion slurries.

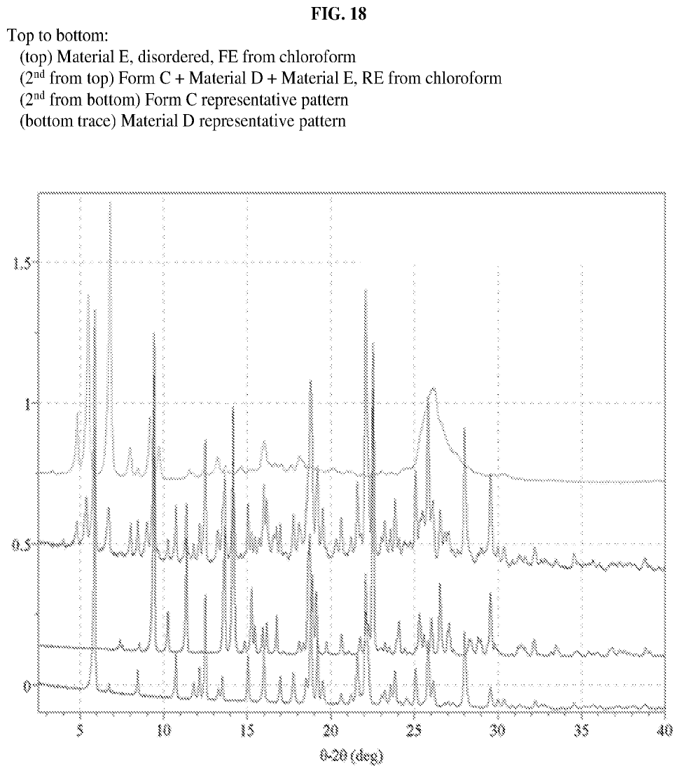

Material E: Material E was observed only as a disordered material from a fast evaporation experiment in chloroform (FIG. 18, top; Table 6). The material was also produced in a mixture with Form C and Material D by rotary evaporation from chloroform (FIG. 18, second from the top; Table 7). The XRPD pattern could not be indexed due to the disorder. A proton NMR spectrum for the material indicated no organic solvent was present at the time of analysis, although desolvation prior to analysis would have been possible, particularly for a material lacking highly crystalline structure (Table 2).

Material F: Material F was observed only as a disordered material from a fast evaporation experiment in TFE (FIG. 3, top; Table 6). The XRPD pattern could not be indexed due to the disorder. A proton NMR spectrum indicated negligible TFE was present at the time of analysis, although, as for Material E, desolvation prior to analysis would have been possible (Table 2). Due to the disorder, the material was not further characterized.

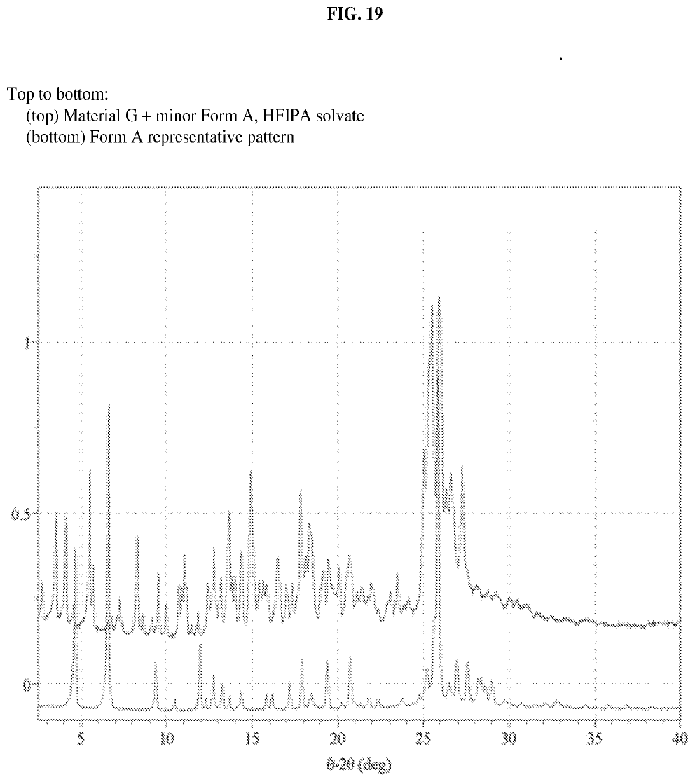

Material G: Material G is an HFIPA solvate (1.9 moles) or mixed solvate/hydrate of Compound 1 that was observed only as a mixture with minor Form A (hydrate, FIG. 19). The mixture resulted from an attempted crash precipitation experiment in HFIPA/water that initially afforded a cloudy liquid with yellow oil, but crystallized to off-white solids after 16 days in the refrigerator. Due to the nature of the mixture, the XRPD pattern could not be indexed.

Proton NMR of the Material G+minor Form A mixture indicated 1.9 moles of HFIPA per mole of API, suggesting a solvate (Table 2).

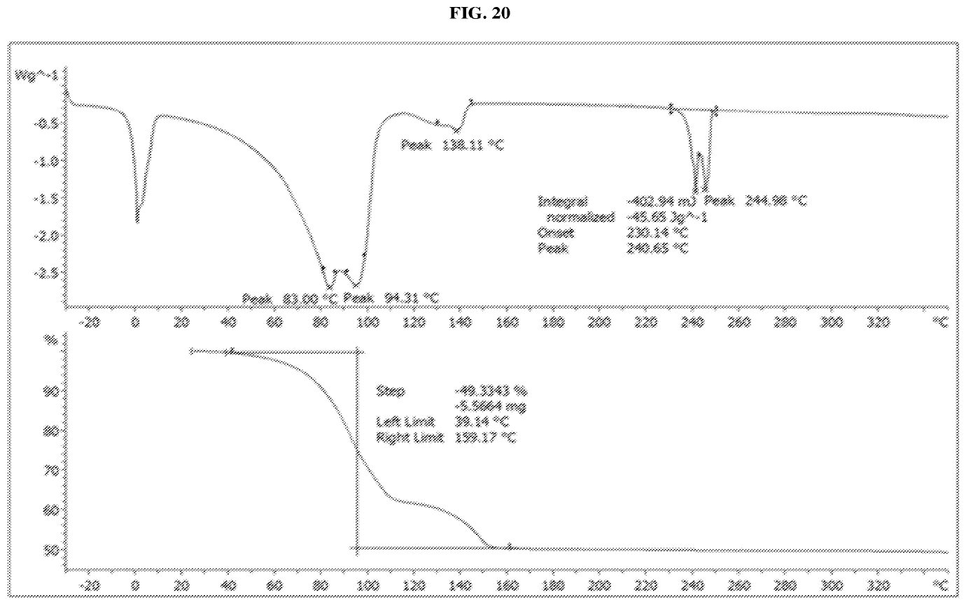

DSC and TGA thermograms for the mixture are shown in FIG. 20. The endotherm at ˜0° C. likely corresponds with melting of residual water. Large weight loss of 49% up to 159° C. corresponds with a series of broad endotherms, consistent with loss of solvent. Overlapping endothermic events are observed at 241° C. and 245° C. (maxima), likely corresponding with melting of a mixture of crystalline forms. The higher melting form is likely Form C, and the lower melting form could be Material D based on the peak maxima.

Material H: Material H is a TFE solvate (0.5 mole TFE) or mixed solvate/hydrate that resulted from a vapor diffusion experiment in TFE/water (FIG. 3, third pattern; Table 6). The XRPD pattern could not be indexed, possibly suggesting a mixture of materials.

Proton NMR of Material H indicated 0.5 mole TFE present, consistent with a likely solvated form (Table 2).

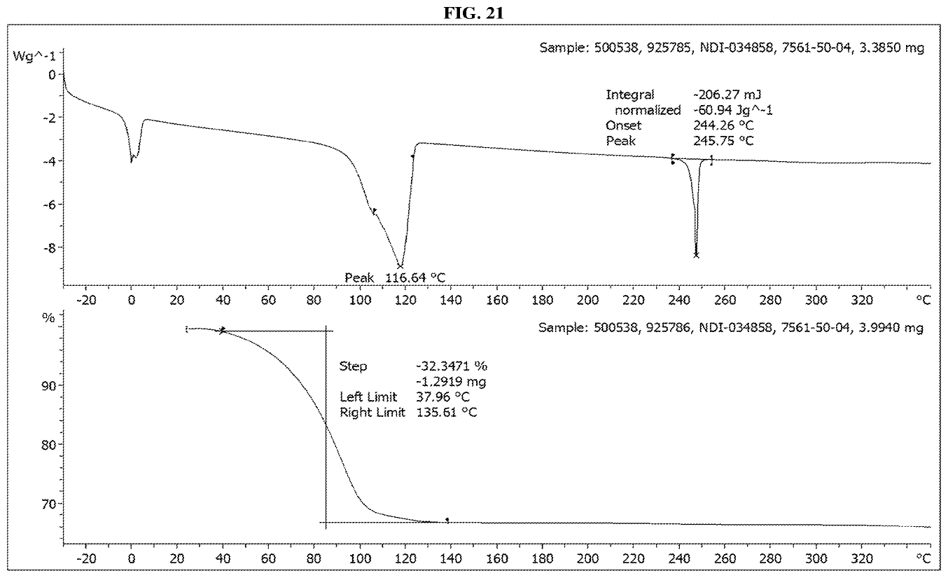

DSC and TGA thermograms are shown in FIG. 21. Stepwise weight loss of 32% up to 136° C. by TGA corresponds with a broad endotherm by DSC, consistent with the loss of solvent. The amount of weight loss is equivalent to over 2 moles of TFE. Since much less TFE was present by proton NMR (0.5 mole), the weight loss is likely due to the loss of both TFE and water. It is unknown whether any water present is bound in the crystal lattice, but at least a portion of the water is residual based on the endotherm at 0° C., indicating melting of the un-bound water. The sharp endotherm at 244° C. (onset) likely corresponds with the melt of the desolvated material.

To study the desolvation behavior, Material H was heated to 150° C. by TGA, and the solids were recovered and analyzed by XRPD (Table 10). Full conversion to Form C was observed.

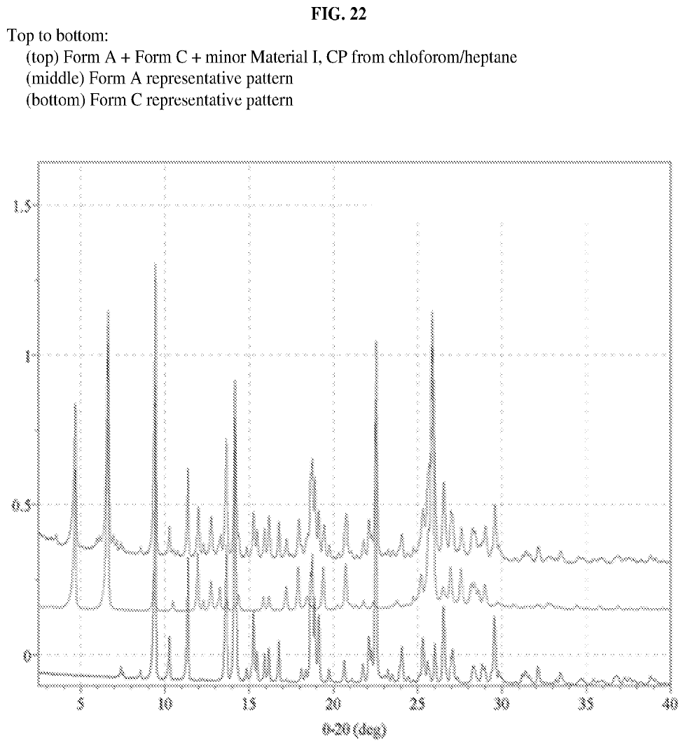

Material I: Material I was observed only as a minor component of a mixture with Forms A and C from a crash precipitation experiment in chloroform/heptane (FIG. 22, Table 6). Due to the nature of the mixture, the material was not further characterized, and the composition remains unknown.

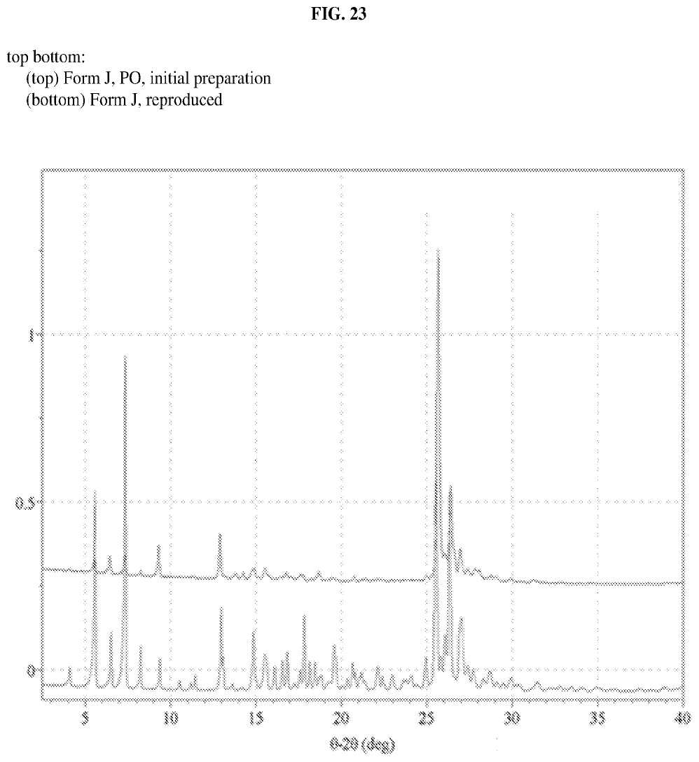

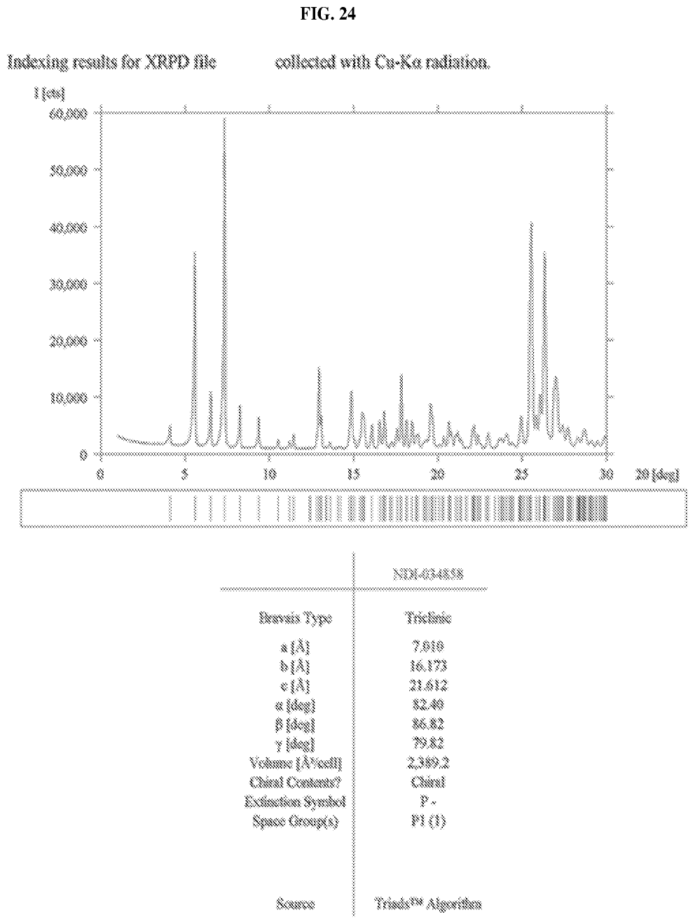

Form J: Form J is a hydrate (1-2 moles water) of Compound 1 that was initially produced in a small quantity from a crash cooling experiment in MeOH (Table 6). The cooling procedure was repeated at a lower cooling rate, with the addition of seeding, to successfully reproduce Form J (Table 11). The XRPD pattern for the initial preparation exhibited preferred orientation effects, which occurs when particles of an anisotropic morphology (such as needles) align in the sample holder causing an amplified signal at some angles and subdued signal at others (FIG. 23, top). Due to the lack of pronounced peaks, this pattern could not be indexed. However, the re-prepared sample exhibited suitable peaks for indexing (FIG. 23, bottom). The unit cell volume could accommodate up to 2 moles of water (FIG. 24).

Proton NMR spectra for both samples indicated negligible MeOH present (Table 2).

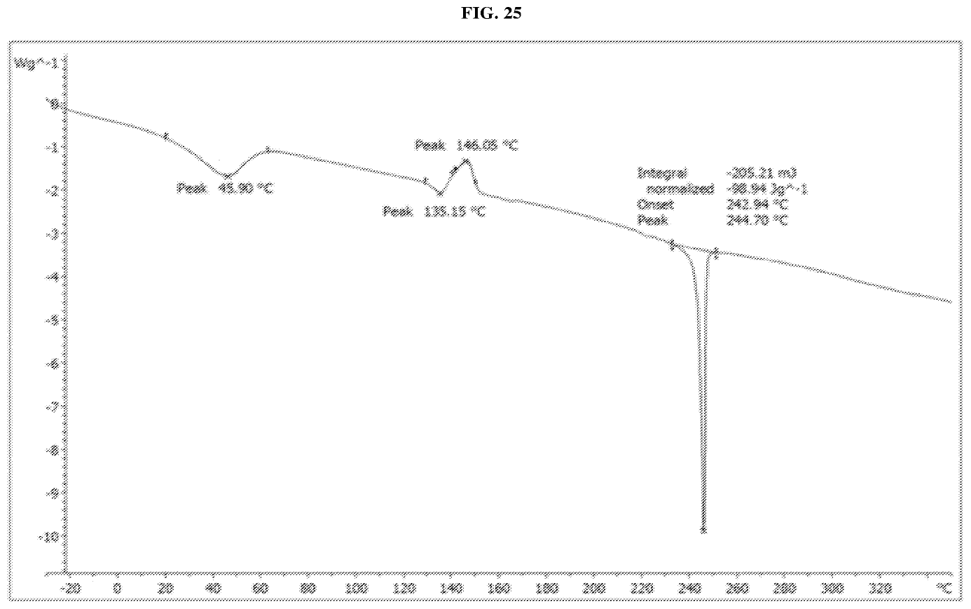

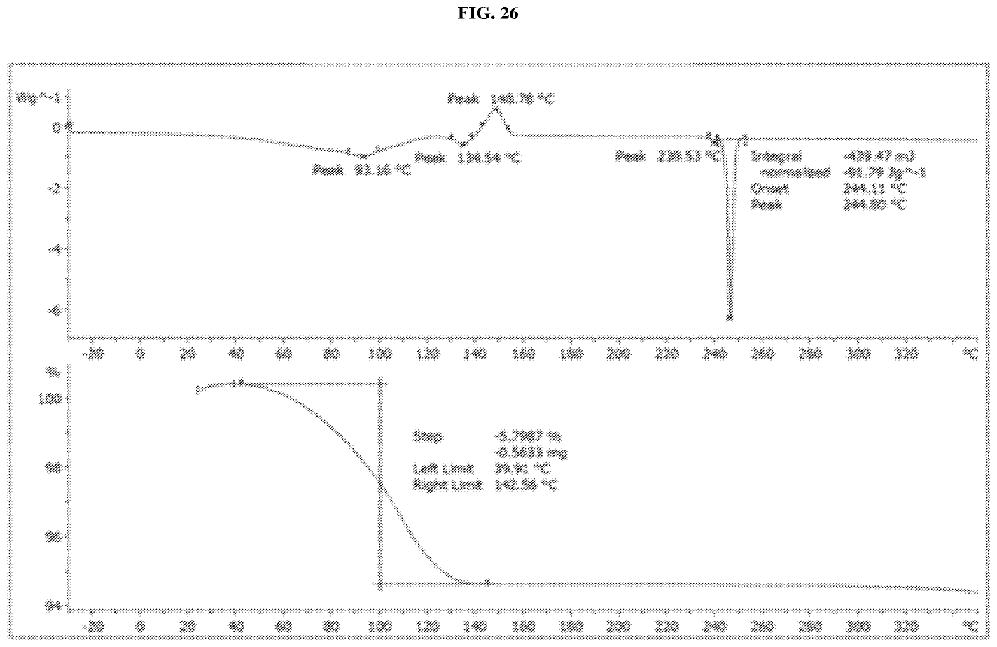

A DSC thermogram for the initial preparation exhibits a broad endotherm at 46° C., possibly indicating loss of water (FIG. 25). Overlapping endothermic and exothermic events at ˜135 to 146° C. likely correspond with recrystallization. The sharp endotherm at 243° C. (onset) is consistent with melting of the recrystallized material, likely Form C based on the onset temperature. Insufficient solids of the initial preparation remained for TGA analysis, so the re-prepared material was analyzed by DSC and TGA (FIG. 26). The DSC thermogram is quite similar to that of the initial preparation. Weight loss of 5.8% by TGA up to 143° C. equates to the loss of the 0.16 mole MeOH detected by proton NMR plus 1.3 moles of water.

Karl Fisher analysis of the material indicated 1.4 moles of water present, consistent with the TGA data (Table 2). Based on the KF, TGA, and indexing results, Form J is reported as a hydrate with 1 to 2 moles of water.

Amorphous: All attempts to prepare amorphous Compound 1 at bench scale, by melt/quench and rotary evaporation, were unsuccessful and resulted in crystalline materials (Table 7). Therefore, a cycling DSC experiment, described supra, was performed in an attempt to perform the melt/quench/reheating in situ to observe a glass transition event (FIG. 15, Table 2). The observation of a glass transition (Tg) can be characteristic of the non-crystalline nature of the material. Form C solids were selected as the starting material due to the anhydrous/non-solvated nature, which would prevent any interference from the loss of solvent or water upon heating. In the second heating leg of the experiment in FIG. 15 (in blue), the material exhibits a Tg at approximately 110° C. (ΔCp: 0.2 J/(g*K)). This Tg is relatively high, a possible indicator of good physical stability of the amorphous solids at ambient temperature. To be noted, differences in solvent and/or water content within the sample can shift the temperature at which the glass transition occurs.

### Relative Thermodynamic Stability of Select Forms/Anhydrous/Non-Solvated Forms

Two anhydrous/non-solvated forms, Form C and Material D, were discovered during the form screen. To evaluate their relative thermodynamic stability, interconversion slurries were conducted at RT and 2 to 8° C. Density values, melt onset temperatures, and heats of fusion are also considered in the discussion of whether the forms transform reversibly at a specific transition temperature (enantiotropic relationship) or if the forms are not interconvertible (monotropic relationship).

To set up the interconversion slurries, THF was pre-saturated with Form C at the stated temperature, and a portion of the liquid phase was filtered into a mixture of solids containing seeds both Form C and Material D (Table 12). A pre-saturated liquid phase is utilized to minimize any kinetic dissolution effects, allowing the less stable (and more soluble) form to dissolve and the most stable (and least soluble) form to precipitate. The slurries were allowed to stir for 7 days at RT and 2 to 8° C., respectively. Both slurries resulted in Form C as a single phase, indicating it is more stable than Material D between 2° C. and RT.

TABLE 12
Slurry Interconversion of Named
Materials and Forms of Compound 1

| Starting | | | | XRPD |
|---|---|---|---|---|
| Materials/Forms | Solvent⁵ | Conditions | Observations | Result |
| Form C, + | THF | RT, 7 d | opaque off- | C |
| Material D, | | | white | |
| | | | suspension; | |
| | | | fines, B/E | |
| Form C + | THF | 2-8° C., | opaque off- | C |
| Material D | | 7 d | white | |
| (presumed), | | | suspension; | |
| | | | fines, B/E | |

TABLE 13
Description of Named Materials and Forms of Compound 1

| SSCI | Previous | Description/ | | |
|---|---|---|---|---|
| Designation | Designation | Composition | Conditions | Characterization |
| Form A | Material A | hydrate (up to 3 moles water) | as-received lot 5 (mixture with | XRPD (indexed), |
| | | dehydrated by ~140° C. | minor Material B) | OM, ¹H NMR, |
| | | converted to Form C upon | slurries at a_{w} ≥ 0.82 | DSC, TGA, DVS, |
| | | dehydration | various polymorph screen | KF |
| | | critical a_{w} between Form A and | experiments, particularly in | |
| | | Form C likely falls between | aqueous solvent systems | |
| | | 0.69-0.82 | | |
| | | significant hygroscopicity (4.58% | | |
| | | sorption 5-95% RH) | | |
| Material B | — | unknown composition | as-received lot 5 (minor | XRPD |
| | | observed only as minor component | component of mixture with | |
| | | of mixture with Form A | Form A) | |
| | | | not observed during form | |
| | | | screen | |
| Form C | Material C | anhydrous/non-solvated | all slurries in organic solvents | SCXRD, XRPD |
| | | likely stable non-solvated form | between 2-80° C. | (indexed), |
| | | between 2-80° C. | slurries in aqueous/organic | ¹H NMR, DSC, |
| | | melt onset 245° C. | mixtures at or below a_{w} 0.69 | TGA, DVS |
| | | limited hygroscopicity (1.75% | numerous polymorph screen | |
| | | sorption 5-95% RH) | experiments | |
| | | | heating of Form A or Material | |
| | | | H to 150° C. | |
| Material D | — | anhydrous/non-solvated | melt/quench/reheat to 200° C. | XRPD (indexed), |
| | | metastable between 2-8° C. and RT | (starting with Form C) | DSC (from cycling |
| | | likely melt onset 238° C. | chloroform, CP w/MTBE (C + | experiment) |
| | | higher density than Form C | D) | |
| | | (1.390 g/cm³ vs 1.324 g/cm³) | RE from chloroform (C + D + E) | |
| Material E | — | observed only as disordered | FE from chloroform | XRPD, ¹H NMR |
| | | material | RE from chloroform (mixture | |
| | | no organic solvent present | with C and D) | |
| Material F | — | observed only as disordered | FE from TFE | XRPD, ¹H NMR |
| | | material | | |
| | | negligible organic solvent present | | |
| Material G | — | HFIPA solvate (1.9 moles) or | HFIPA, CP w/water (yellow | XRPD, ¹H NMR, |
| | | mixed solvate/hydrate | oil), stand in ref 16 d | DSC, TGA |
| | | observed only as mixture w/minor | | |
| | | Form A | | |
| | | desolvated by 159° C. | | |
| | | likely converted to mixture of | | |
| | | Form C and Material D upon | | |
| | | desolvation | | |
| Material H | — | TFE solvate (0.5 mole) or mixed | TFE, VD w/water | XRPD, ¹H NMR, |
| | | solvate/hydrate | | DSC, TGA |
| | | desolvated by 136° C. | | |
| | | converted to Form C upon | | |
| | | desolvation | | |
| Material I | — | unknown composition | chloroform, CP w/MTBE | XRPD |
| | | observed only as minor component | | |
| | | of mixture with Forms A and C | | |
| Form J | Material J | hydrate (1-2 moles water) | cooling from MeOH in frz | XRPD (indexed), |
| | | dehydrated by ~143° C. | | ¹H NMR, DSC, |
| | | likely converted to Form C upon | | TGA, KF |
| | | dehydration | | |

⁵ Liquid phase for each slurry was pre-saturated with Compound 1 using various Form C samples. Saturated solution (THF at RT) and (THF at 2-8° C.).

The density rule, which is based on Kitaǐgorodskiǐ's principle of closest packing for molecular crystals, states that, for a non-hydrogen-bonded system at absolute zero, the most stable polymorph will have the highest density because of stronger intermolecular van der Waals interactions. As discussed supra, Material D exhibited higher density (1.390 g/cm³, from XRPD indexing) than Form C (1.324 g/cm³, from SCXRD). If the density rule can be applied to this system, these values would indicate that Material D is more thermodynamically stable than Form C between absolute zero and an undetermined transition temperature below 2° C. (enantiotropic relationship).

In addition to density values, melting onset temperatures and heats of fusion, measured by DSC, can be compared between anhydrous/non-solvated materials to further understand their thermodynamic relationship (monotropic versus enantiotropic). From the heat of fusion rule, two forms are enantiotropic if the higher melting form has the lower heat of fusion; otherwise, they are monotropic. The cycling DSC thermogram shown in FIG. 15 exhibits melting endotherms for both Form C (first leg of experiment, in black) and presumed Material D (third leg of experiment, in blue). In this data, the melt onset and heat of fusion for Form C (245° C., 67 J/g) are both higher than those of Material D (238° C., 65 J/g), which would indicate a monotropic relationship in which Form C is more thermodynamically stable than Material D at all temperatures. However, one must consider the unknown degree of crystallization for Material D in this experiment and how that could impact the heat of fusion value. Overall, Form C was shown to be more stable than Material D between 2° C. and RT.

### Anhydrous Versus Hydrated Forms

Slurry experiments at various water activities were conducted to study the relationship between anhydrous/non-solvated Form C and hydrate Form A at RT. From the stable form slurries in select acetone/water, MeOH/water, and THF/water mixtures, Form A was found to fully or partially convert to Form C at or below aw 0.69 (Table 5). Form A remained unchanged at or above aw 0.82, likely indicating the critical water activity between Form A and Form C falls between 0.69 and 0.82 at RT. To be noted, the RT slurry at aw 0.82 was stirred for only 2 days prior to isolation (due to investigation of visible color changes). Since this slurry started with solids consisting primarily of Form A and resulted in Form A, it is possible that insufficient time was allowed for form conversion. All other slurries of Form A at various water activities were stirred for at least 10 days, likely allowing sufficient time for form conversion.

In addition to the slurries starting with Form A, two slurries of Form C were set up at high water activity conditions to study possible conversion to a hydrate (Table 9, supra). THF/water 50:50 (aw ˜1) and neat water (aw 1) were used. Full conversion to Form A was observed after 1 day in THF/water 50:50, while no form conversion was noted after 1 day in neat water, likely due to insufficient solubility.

Overall, the data indicate that Form C is likely the thermodynamically stable form at or below aw 0.69 and Form A is likely favored at or above aw 0.82. Form C can convert to Form A relatively quickly upon slurrying above the critical water activity (providing suitable solubility for form conversion is achieved), although Form C did not readily convert to a hydrate in the solid state at ˜90% RH (stressed for 11 days, discussed supra, Table 8).

Due to material limitations, hydrate Form J was not included in the relative thermodynamic stability studies at various water activities.

### CONCLUSIONS AND SUMMARY

Compound 1 mixture (designated as a mixture, Form A+minor Material B) was received for use in a polymorph screen. Approximately 50 screening experiments were set up, employing a variety of crystallization techniques and solvent systems. The compound exhibited a relatively high propensity to exist in various solid forms, with eight new materials discovered during the screen (Materials/Forms C through J). Forms of interest include anhydrous/non-solvated Form C and Material D and hydrated Forms A and J. All other named materials are solvated, disordered, or were observed only in mixtures.

Form C is the likely thermodynamically stable anhydrous/non-solvated form between 2° C. and 80° C., while Material D is metastable within that temperature range.

The critical water activity between hydrate Form A and anhydrous Form C likely falls between 0.69 and 0.82. Due to material limitations, hydrate Form J was not included in any water activity studies.

Based on the body of work conducted, Form C is recommended for further pursuit. While Form A can readily convert to Form C by slurrying below the critical water activity, care should be taken to provide suitable conditions (solubility, temperature, water activity, seeding, etc.) for complete conversion to the desired form. A crystallization process development study would help to optimize the experimental parameters for a robust procedure to reproducibly crystallize Form C.

### Experimental Procedures

Procedures for Compound 1 Form C: THF slurry, solids from Compound 1 (Form A+minor Material B, 62.2 mg) were combined with THF (1 mL). The resulting slurry was allowed to stir at RT for 2 days, resulting in an opaque green suspension (experiment was done prior to the discovery of potential light sensitivity of the compound). Solids were collected on a 0.2-μm nylon filter by positive-pressure filtration, flushed with ˜20 mL of air 10 times, and transferred to a clean vial. EtOAc slurry at ˜60° C. Solids from Compound 1 (Form A+minor Material B, 67.5 mg) were combined with EtOAc (2 mL). The resulting slurry was allowed to stir in a metal heater block on a hot plate at 460° C. for 3 days, resulting in an opaque off-white suspension. While warm, solids were collected on a 0.2-μm nylon filter by positive-pressure filtration, flushed with ˜20 mL of air 10 times, and transferred to a clean vial.

### Experimental Techniques

Observations of color changes, typically to green, among the initial set of slurry experiments within a few days lead to the treatment of all later samples as light sensitive. This includes handling samples primarily in a fume hood with the lights turned off or otherwise shielding experiments from long-term light exposure, such as by covering with foil.

### Isolation Techniques

In general, isolation of solids was done quickly after removing non-ambient samples from their respective temperature control devices to minimize equilibration to ambient temperature.

### Decanting Liquid Phase

For some non-homogeneous slurries, solids were isolated by centrifuging the suspension (if needed) and discarding the liquid phase, leaving behind damp solids. Solids were dried briefly (e.g., air dried or under nitrogen) unless specified as “analyzed damp” in data table infra.

### Positive-Pressure Filtration

Solids were collected on 0.2-μm nylon or PTFE filters by pressing a slurry through a syringe and Swinnex filter holder assembly. In general, solids were dried briefly by blowing a 20-mL syringe of air over the filter several times. If designated as “analyzed damp” infra, solids were left damp with mother liquor. Some samples were additionally dried briefly under a gentle stream of nitrogen gas prior to analysis.

### Vacuum Filtration

Solids were collected on paper or nylon filters by vacuum filtration and air dried on the filters under reduced pressure briefly before transferring to a vial.

### Crash Cool (CC)

Concentrated solutions were prepared in various solvents at an elevated temperature and, typically, filtered warm through a 0.2-μm nylon or PTFE filter into a warm vial. Each solution was capped and then immediately cooled to sub-ambient temperature, such as by placing in a freezer or plunging into a bath of dry ice and isopropanol. Solutions were allowed to remain at the sub-ambient temperature for a stated amount of time, and any solids present were isolated as described above. If no solids were observed or if oily materials resulted, additional techniques were employed if specified.

### Crash Precipitation (CP)

Solutions were prepared in various solvents and, typically, filtered through a 0.2-μm nylon or PTFE filter. Aliquots of various antisolvents were dispensed with stirring until precipitation occurred. If necessary, samples were placed at sub-ambient temperatures to facilitate precipitation or crystallization. Solids were isolated as described above. If no solids were observed or if oily materials resulted, additional techniques were employed if specified.

### Fast Evaporation (FE)

Solutions were prepared in various solvents and, typically, filtered through a 0.2-μm nylon or PTFE filter. Each solution was allowed to evaporate from an open vial at ambient conditions, unless otherwise stated. Solutions were allowed to evaporate to dryness unless designated as partial evaporations (solid present with a small amount of solvent remaining), in which case solids were isolated as described above.

### Interconversion Slurries

A saturated solution was prepared by adding enough solids of a given form to a given solvent system at stated conditions so that undissolved solids were present. The mixture was then agitated or allowed to stand at a stated temperature for an extended period of time to ensure saturation. Seeds of the forms of interest were added to aliquots of the saturated solution (filtered through a 0.2-μm nylon or PTFE filter) so that undissolved solids were present. The mixture was then agitated in a sealed vial at the stated temperature for an extended period of time. The solids were isolated as described above.

### Relative Humidity Stressing

Select materials were transferred to a vial, which was then uncapped and placed inside ajar containing a saturated aqueous barium chloride solution for ˜90% RH in the headspace (See, Nyqvist, H. E., Saturated Salt Solutions for Maintaining Specified Relative Humidities, Int. J. Pharm. Technol. Prod. Manuf 1983, 4, 47-48, which is incorporated by reference in its entirety). Jars were stored at the stated temperatures.

### Rotary Evaporation (RE)

Solutions were prepared in stated solvents and, typically, filtered through a 0.2 μm nylon or PTFE filter. The solution was evaporated to dryness using a rotary evaporator at the stated temperature. Resulting solids were stored at stated conditions.

### Slow Cool (SC)

Concentrated solutions were prepared in various solvents at an elevated temperature and, typically, filtered warm through a 0.2-μm nylon or PTFE filter into a warm vial. Each solution was capped and left on the hot plate, and the hot plate was turned off to allow the sample to slowly cool to ambient temperature. If no solids were present after cooling to ambient temperature, the sample was further cooled at sub-ambient temperatures. Any solids present after cooling were isolated as described above.

### Slow Evaporation (SE)

Solutions were prepared in various solvents and, typically, filtered through a 0.2-μm nylon or PTFE filter. Each solution was allowed to evaporate from a covered vial (such as loosely capped or covered with perforated aluminum foil) at ambient conditions. Solutions were allowed to evaporate to dryness unless designated as partial evaporations (solid present with a small amount of solvent remaining), in which case solids were isolated as described above.

### Slurry Experiments

Suspensions were prepared by adding enough solids to a given solvent at the stated conditions so that undissolved solids were present. The mixture was then agitated (typically by stirring or oscillation) in a sealed vial at a given temperature for an extended period of time. The solids were isolated as described above.

### Solubility Approximation

Aliquots of various solvents were added to measured amounts of Compound 1 with agitation (typically sonication) at stated temperatures until complete dissolution was achieved, as judged by visual observation. If dissolution occurred after the addition of the first aliquot, values are reported as “>”. If dissolution did not occur, values are reported as “<”. Solubility values are reported as approximate (“˜”) if the majority of solids dissolved while a few undissolved particles persisted (likely due to a minor impurity in the as-received lot).

### Vapor Diffusion (VD)

Concentrated solutions were prepared in various solvents and, typically, filtered through a 0.2 μm nylon or PTFE filter. The filtered solution was dispensed into a small vial, which was then placed inside a larger vial containing antisolvent. The small vial was left uncapped, and the larger vial was capped to allow vapor diffusion to occur. Any solids present were isolated as described above.

### Computational Techniques

### XRPD Indexing

Indexing and structure refinement are computational studies. Within the figure referenced for a given indexed XRPD pattern, agreement between the allowed peak positions, marked with red bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below the figure. To confirm the tentative indexing solution, the molecular packing motifs within the crystallographic unit cells must be determined. No attempts at molecular packing were performed.

The following patterns were indexed using proprietary SSCI software (TRIADS™ is covered by U.S. Pat. No. 8,576,985, which is incorporated by reference in its entirety): Form A, Form C, Form D and Form J.

### Instrumental Techniques

### Differential Scanning Calorimetry (DSC)

DSC was performed using a Mettler-Toledo DSC3+ or DSC 822e differential scanning calorimeter. Temperature calibration was performed using octane, phenyl salicylate, indium, tin, and zinc. The sample was placed into a hermetically sealed aluminum DSC pan, the weight was accurately recorded, the lid was pierced, and the sample was inserted into the DSC cell. A weighed aluminum pan configured as the sample pan was placed on the reference side of the cell. The samples were analyzed from −30° C. to 350° C. at 10° C./min.

### Dynamic Vapor Sorption (DVS)

Moisture sorption/desorption data were collected on a Surface Measurement System DVS Intrinsic instrument. The samples were not dried prior to analysis. Sorption and desorption data were collected over a range from 5% to 95% RH in 10% RH increments under a nitrogen purge. The equilibrium criteria used for the analyses were 0.001 dm/dt weight change in 5 minutes with a minimum step time of 30 minutes and maximum equilibration time of 180 minutes with a 3-minute data logging interval. Data were not corrected for the initial moisture content of the samples.

### Karl Fischer Titration (KF)

Coulometric Karl Fischer analysis for water determination was performed using a Mettler Toledo DL39 Karl Fischer titrator with a Stromboli oven attachment. A NIST-traceable water standard (Hydranal Water Standard 1.0) was analyzed to check the operation of the coulometer. Additionally, a qualified standard (Apura Water Standard Oven 1%) was analyzed to check the operation of the coulometer/oven system. Approximately 5-40 mg of sample was weighed in a pre-dried Stromboli vial and sealed. Two samples were weighed and placed into the drying oven set at ˜160° C. (only one replicate was performed for LIMS 502283 due to sample limitations). The drying oven was purged into the titrator vessel with dry nitrogen. The samples were then titrated by means of a generator electrode, which produces iodine by electrochemical oxidation: 2I−→I2+2c−.

### Optical Microscopy

Samples were observed under a Motic or Wolfe optical microscope with crossed polarizers or under a Leica stereomicroscope with a first order red compensator with crossed polarizers.

### Solution ¹H NMR Spectroscopy

The solution ¹H NMR spectra were acquired by Spectral Data Services of Champaign, IL. The samples were dissolved in DMSO-d6. The data acquisition parameters are displayed on the first page of each spectrum in the Data section of this report.

### Thermogravimetry (TGA)

TG analysis was performed using a Mettler-Toledo TGA/DSC3+ analyzer. Temperature calibration was performed using calcium oxalate, indium, tin, and zinc. The sample was placed in an aluminum pan. The pan was hermetically sealed, the lid pierced, then inserted into the TG furnace. A weighed aluminum pan configured as the sample pan was placed on the reference platform. The furnace was heated under nitrogen. Samples were analyzed from 25° C. to 350° C. at 10° C./min. The TGA/DSC3+ instrument was also used for select heating/desolvation experiments, in which solids were heated as described to a given temperature, then recovered and analyzed by XRPD.

### X-Ray Powder Diffraction (XRPD)

XRPD patterns were collected with a PANalytical X'Pert PRO MPD or a PANalytical Empyrean diffractometer using an incident beam of Cu radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 2.2b or 5.5. The data acquisition parameters are listed in the Figures.

### Single Crystal X-ray Diffraction (SCXRD)

Standard uncertainty in this report is written in crystallographic parenthesis notation, e.g., 0.123(4) is equivalent to 0.123±0.004.

Preparation of the Single Crystal Sample: Solids from as-received Compound 1 (64.6 mg) were combined with MEK (2 mL), and the resulting slurry was allowed to stir in a metal heater block on a hot plate at ˜60° C. for 3 days. The sample consisted primarily of an opaque off-white suspension with a few amber and light green solids on the upper walls. Solids on the walls were observed to be rectangular plates by optical microscopy. A suitable single crystal was culled and analyzed.

### Data Collection

A light-yellow block having approximate dimensions of 0.6×0.26×0.07 mm³, was mounted on a polymer loop in random orientation. Preliminary examination and data collection were performed on a Rigaku SuperNova diffractometer, equipped with a copper anode microfocus sealed X-ray tube (Cu θ=1.54184 Å) and a Dectris Pilatus3 R 200K hybrid pixel array detector.

Cell constants and an orientation matrix for data collection were obtained from least-squares refinement using the setting angles of 16314 reflections in the range 3.69200<<77.3870°. The space group was determined by the program CRYSALISPRO (CrysAlisPro 1.171.38.41r (Rigaku Oxford Diffraction, 2015)) to be P21 (international tables no. 4). The data were collected to a maximum diffraction angle (2θ) of 155.2340 at room temperature.

### Data Reduction

Frames were integrated with CRYSALISPRO. A total of 25050 reflections were collected, of which 9477 were unique. Lorentz and polarization corrections were applied to the data. The linear absorption coefficient is 0.758 mm⁻¹ for Cu Kα radiation. An empirical absorption correction using CRYSALISPRO was applied. Transmission coefficients ranged from 0.863 to 1.000. A secondary extinction correction was applied. The final coefficient, refined in least-squares, was 0.0016(3) (in absolute units). Intensities of equivalent reflections were averaged. The agreement factor for the averaging was 2.37% based on intensity.

### Structure Solution and Refinement

The structure was solved by charge flipping using OLEX2 (Bourhis, L. J., Dolomanov, O. V., Gildca, R. J., Howard, J. A. K., Puschmann, H. Acta Cryst., 2015, A71, 59-75, which is incorporated by reference in its entirety). The remaining atoms were located in succeeding difference Fourier syntheses. The structure was refined using SHELXL-2014 (Sheldrick, G. M. Acta Cryst., 2008, A64, 112-122 and Sheldrick, G. M. Acta Cryst. 2015, A71, 3-8, each of which is incorporated by reference in its entirety). Hydrogen atoms residing on nitrogen were refined independently. Hydrogen atoms residing on carbon were included in the refinement but restrained to ride on the atom to which they are bonded. The structure was refined in full-matrix least-squares by minimizing the function:

$$\sum {w ( {\vert {F}_{o} \vert}^{2} - {\vert {F}_{c} \vert}^{2} )}^{2}$$

where the weight, w, is defined as 1/[σ²(Fₒ²)+(0.0716P)²+(0.2864P)], where P=(Fₒ²+2Fₑ²)/3.

Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4, which is incorporated by reference in its entirety). Of the 9477 reflections used in the refinements, only the reflections with intensities larger than twice their uncertainty [I>2σ(I)], 8578, were used in calculating the fit residual, R. The final cycle of refinement included 642 variable parameters, 1 restraint, and converged with respective unweighted and weighted agreement factors of:

$$R = \sum \vert {F}_{o} - {F}_{c} \vert / \sum {F}_{o} = 0 \text{.0445}$$

$${R}_{w} = \sqrt{( \sum {w ( {F}_{o}^{2} - {F}_{c}^{2} )}^{2} / \sum {w ( {F}_{o}^{2} )}^{2} )} = 0.1238$$

The standard deviation of an observation of unit weight (goodness of fit) was 1.08. The highest peak in the final difference Fourier had an electron density of 0.505 e/Å3. The minimum negative peak had a value of −0.325 e/Å3.

### Calculated X-Ray Powder Diffraction (XRPD) Pattern

A calculated XRPD pattern was generated for Cu radiation using MERCURY (Macrae, C. F. Edgington, P. R. McCabe, P. Pidcock, E. Shields, G. P. Taylor, R. Towler M. and van de Streek, J. J. Appl. Cryst., 2006, 39, 453-457, which is incorporated by reference in its entirety) and the atomic coordinates, space group, and unit cell parameters from the single crystal structure.

### Atomic Displacement Ellipsoid and Packing Diagrams

The atomic displacement ellipsoid diagram was prepared using MERCURY. Atoms are represented by 50% probability anisotropic thermal ellipsoids.

While we have described a number of embodiments, it is apparent that our basic examples may be altered to provide other embodiments that utilize the compounds and methods described herein. Therefore, it will be appreciated that the scope of this invention is to be defined by the appended claims rather than by the specific embodiments that have been represented by way of example.

### REFERENCES (EACH OF WHICH IS INCORPORATED BY REFERENCE IN ITS ENTIRETY)

1. Hofmann, D. W. M. Acta Crystallographica Section B: Structural Science. 2002, B57, 489-493.
2. Glusker, Jenny Pickworth; Trueblood, Kenneth N. Crystal Structure Analysis: A Primer, 2^{nd} ed.; Oxford University press: New York, 1985; p. 87.
3. Kitaigorodskii, A. I. Molecular Crystals and Molecules; Academic Press: New York, 1973.
4. Burger, A.; Ramberger, R. On the polymorphism of pharmaceuticals and other organic molecular crystals. I: Theory of thermodynamic rules. Mikrochim. Acta [Wein]1979 II, 259-271.
5. SSCI internal report, Water Activity Calculations using UNIFAC Calculator, SR-20150515.01, dated Jul. 23, 2015.
6. Nyqvist, H. E., Saturated Salt Solutions for Maintaining Specified Relative Humidities, Int. J. Pharm. Technol. Prod. Manuf. 1983, 4, 47-48.
7. Goldberg, R. N.; Nuttall, R. L., Evaluated Activity and Osmotic Coefficients for Aqueous Solutions: The Alkaline Earth Metal Halides, J. Phys. Chem. Ref. Data, 1978, 7 (1), 263-310.
8. TRIADS™ is covered by U.S. Pat. No. 8,576,985.
9. CrysAlisPro 1.171.38.41r (Rigaku Oxford Diffraction, 2015).
10. Bourhis, L. J., Dolomanov, O. V., Gildea, R. J., Howard, J. A. K., Puschmann, H. Acta Cryst., 2015, A71, 59-75.
11. Sheldrick, G. M. Acta Cryst., 2008, A64, 112-122.
12. Sheldrick, G. M. Acta Cryst. 2015, A71, 3-8.
13. International Tables for Crystallography, Vol. C, Kluwer Academic Publishers: Dordrecht, The Netherlands, 1992, Tables 4.2.6.8 and 6.1.1.4.
14. Macrae, C. F. Edgington, P. R. McCabe, P. Pidcock, E. Shields, G. P. Taylor, R. Towler M. and van de Streek, J. J. Appl. Cryst., 2006, 39, 453-457.
15. Hygroscopicity terms and definitions developed by SSCI are based in part on concepts presented in the following: Newman, A. W.; Reutzel-Edens, S. M.; Zografi, G. Characterization of the “Hygroscopic” Properties of Active Pharmaceutical Ingredients, J. Pharm. Sci. 2008, 97, 1047-1059.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

We claim:

1. A solid form of Compound 1: 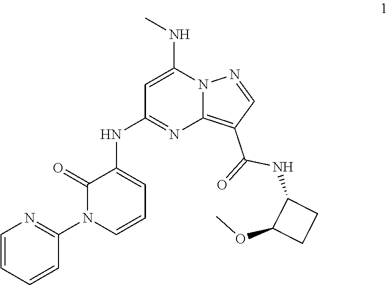 [SMILES: CNc1cc(Nc2cccn(-c3ccccn3)c2=O)nc2c(C(=O)N[C@@H]3CC[C@H]3OC)cnn12]
wherein the solid form is of Form C.

2. The solid form of claim 1, wherein the solid form is substantially free of impurities.

3. The solid form of claim 1 or 2, wherein the solid form is a crystalline solid substantially free of amorphous Compound 1.

4. The solid form of any one of claims 1-3, further characterized by having at least 3, 4, 5, or 6 peaks of an X-ray powder diffraction pattern (XRPD) of FIG. 16 (bottom trace).

5. The solid form of claim 4, further characterized by having at least 3 of the highest amplitude peaks of the XRPD of FIG. 16 (bottom trace).

6. The solid form of claim 4, further characterized by having at least 4 of the highest amplitude peaks of the XRPD of FIG. 16 (bottom trace).

7. The solid form of claim 4, further characterized by having at least 5 of the highest amplitude peaks of the XRPD of FIG. 16 (bottom trace).

8. The solid form of any one of claims 1-3, further characterized by having at least 3, 4, 5, or 6 peaks of an X-ray powder diffraction pattern (XRPD) of FIG. 10.

9. The solid form of claim 8, further characterized by having at least 3 of the highest amplitude peaks of the XRPD of FIG. 10 (top trace).

10. The solid form of claim 8, further characterized by having at least 4 of the highest amplitude peaks of the XRPD of FIG. 10 (top trace).

11. The solid form of claim 8, further characterized by having at least 5 of the highest amplitude peaks of the XRPD of FIG. 10 (top trace).

12. The solid form of any one of claims 1-3, wherein the solid form exhibits an X-ray powder diffraction pattern (XRPD) that is substantially similar to that of FIG. 10 (top trace) or FIG. 16 (bottom trace).

13. The solid form of any one of claims 1-12, further characterized by a TGA analysis of mass loss between 165° C. and 175° C.

14. The solid form of any one of claims 1-13, further characterized by a DSC comprising a peak onset at about 247° C.

15. The solid form of any one of claims 1-13, further characterized by a DSC substantially as shown in FIG. 12.

16. The solid form of any one of claims 1-13, further characterized by a DVS isotherm substantially as shown in FIG. 13.

17. A solid form of Compound 1: 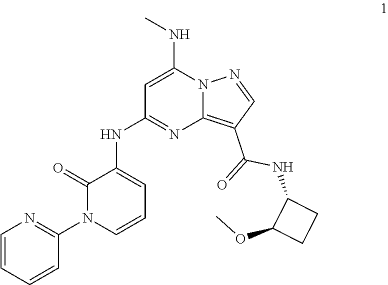 [SMILES: CNc1cc(Nc2cccn(-c3ccccn3)c2=O)nc2c(C(=O)N[C@@H]3CC[C@H]3OC)cnn12]
wherein the solid form is of Form A.

18. The solid form of claim 17, wherein the solid form is substantially free of impurities.

19. The solid form of claim 17 or 18, wherein the solid form is a crystalline solid substantially free of amorphous Compound 1.

20. The solid form of any one of claims 17-19, wherein the solid form is a hydrate.

21. The solid form of claim 20, wherein the solid form is a monohydrate.

22. The solid form of claim 20, wherein the solid form is a dihydrate.

23. The solid form of claim 20, wherein the solid form is a trihydrate.

24. The solid form of any one of claims 17-23, further characterized by having at least 3, 4, 5, or 6 peaks of an X-ray powder diffraction pattern (XRPD) of FIG. 4 (bottom trace) or FIG. 5.

25. The solid form of claim 24, further characterized by having at least 3 of the highest amplitude peaks of the XRPD of FIG. 4 (bottom trace) or FIG. 5.

26. The solid form of claim 24, further characterized by having at least 4 of the highest amplitude peaks of the XRPD of FIG. 4 (bottom trace) or FIG. 5.

27. The solid form of claim 24, further characterized by having at least 5 of the highest amplitude peaks of the XRPD of FIG. 4 (bottom trace) or FIG. 5.

28. The solid form of any one of claims 17-23, characterized by having an X-ray powder diffraction pattern (XRPD) that is substantially similar to that of FIG. 4 (bottom trace) or FIG. 5.

29. The solid form of claim 24, further characterized by having at least 3 of the highest amplitude peaks of the XRPD of FIG. 5.

30. The solid form of claim 24, further characterized by having at least 4 of the highest amplitude peaks of the XRPD of FIG. 5.

31. The solid form of claim 24, further characterized by having at least 5 of the highest amplitude peaks of the XRPD of FIG. 5.

32. The solid form of any one of claims 17-31, further characterized by a TGA analysis of mass loss between 65° C. and 130° C.

33. The solid form of claim 32, wherein the TGA is substantially as shown in FIG. 7.

34. The solid form of any one of claims 17-33, characterized by a DSC comprising peak onsets at about 91° C., 103° C., and 245° C.

35. The solid form of claim 34, wherein the peak onset consists essentially of 245° C.

36. The solid form of claim 34, characterized by a DSC substantially as shown in FIG. 7.

37. A solid form of Compound 1: 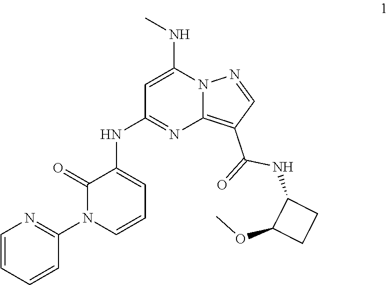 [SMILES: CNc1cc(Nc2cccn(-c3ccccn3)c2=O)nc2c(C(=O)N[C@@H]3CC[C@H]3OC)cnn12]
characterized in that the solid form is of Form A, Form B, Form C, Form D, Form E, Form F, Form G, Form H, Form I, or Form J.

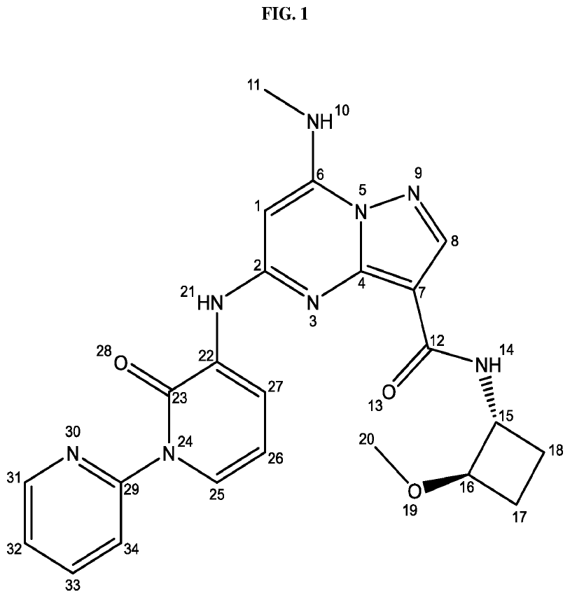

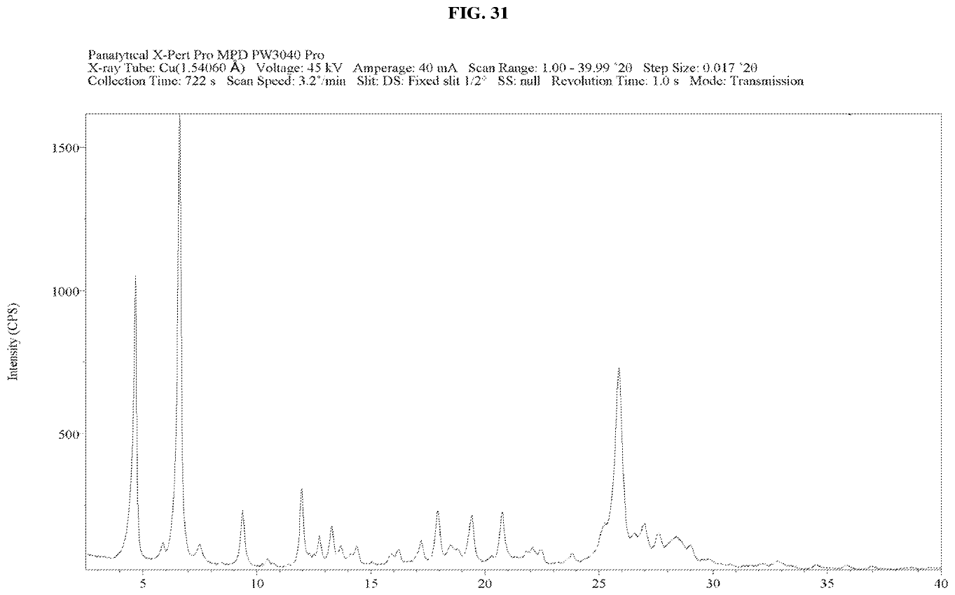

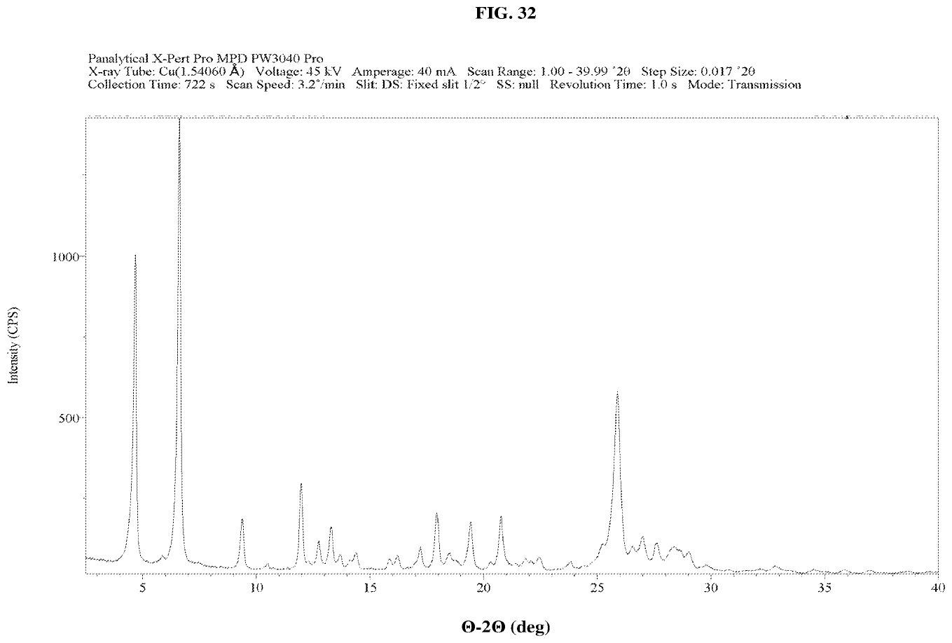

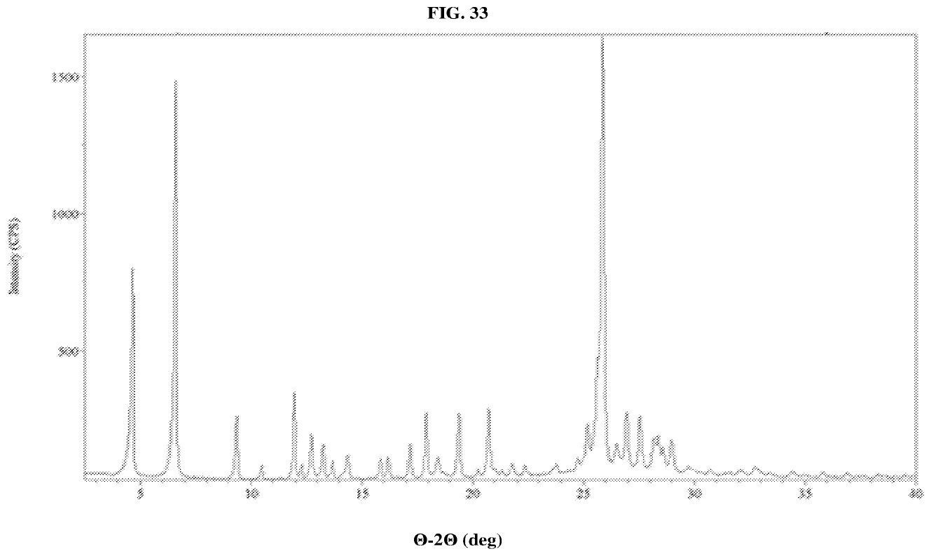

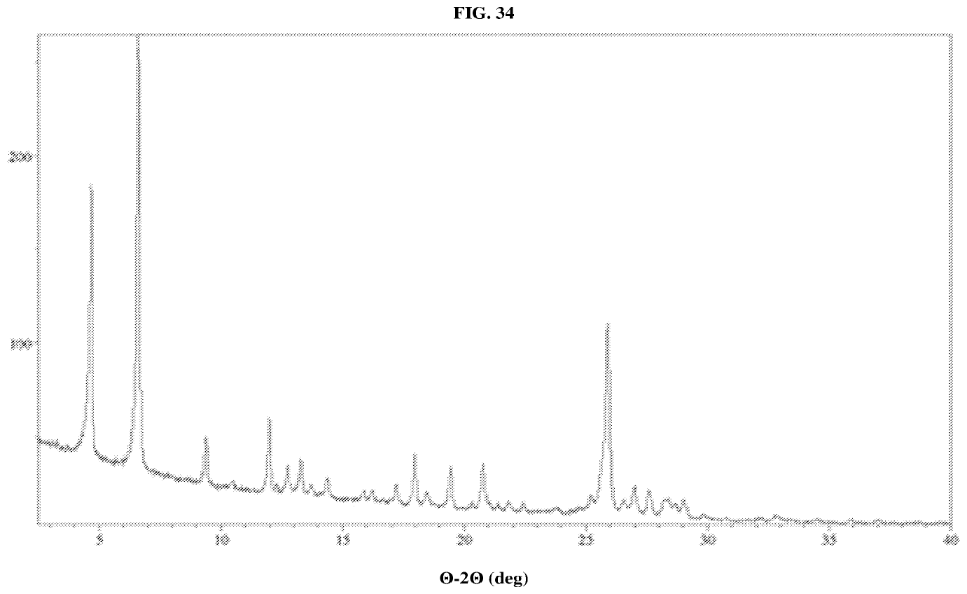

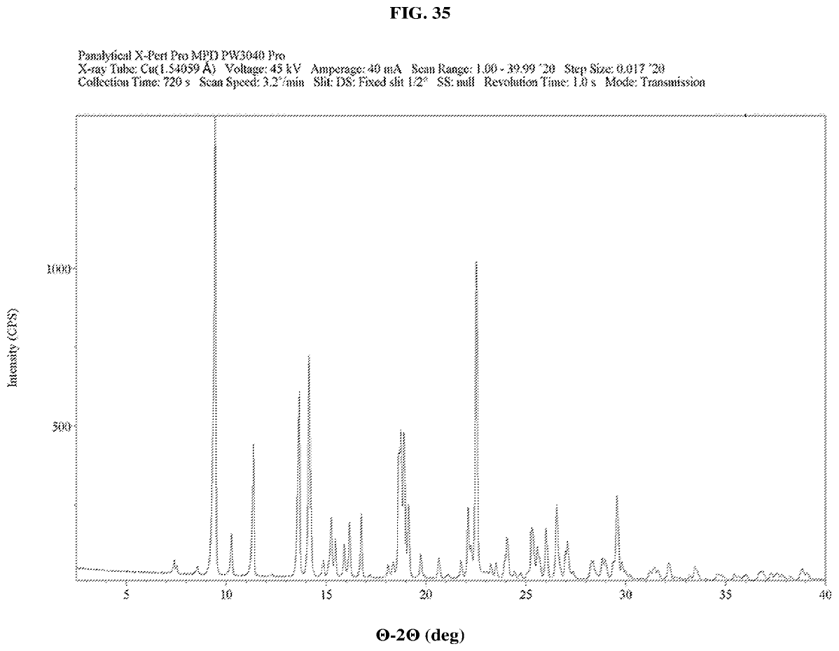

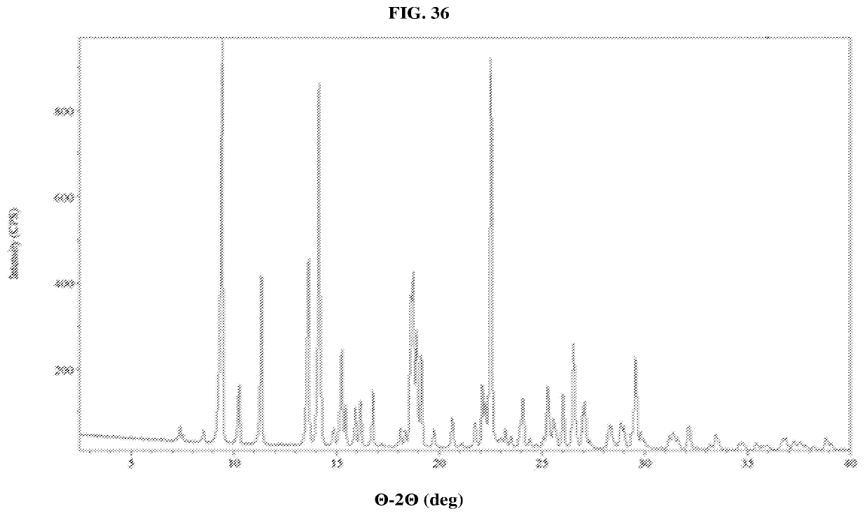

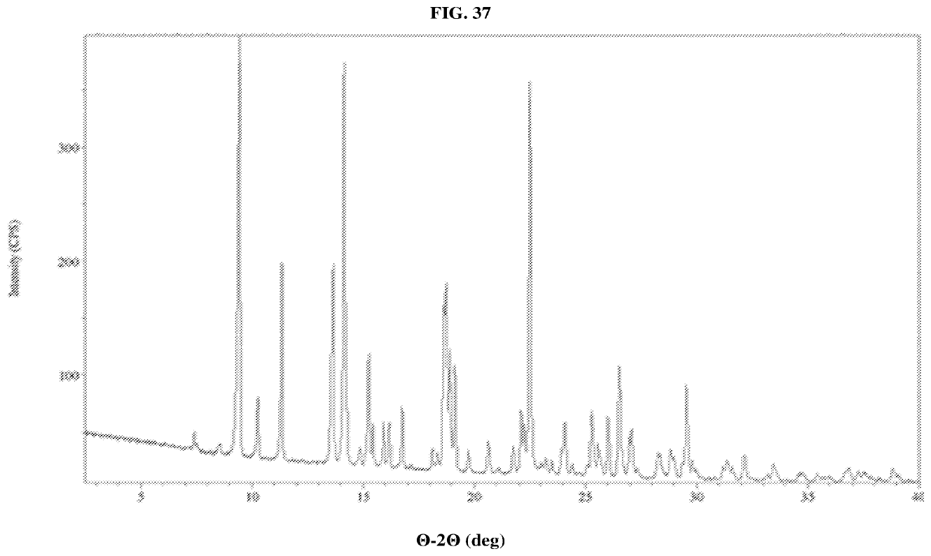

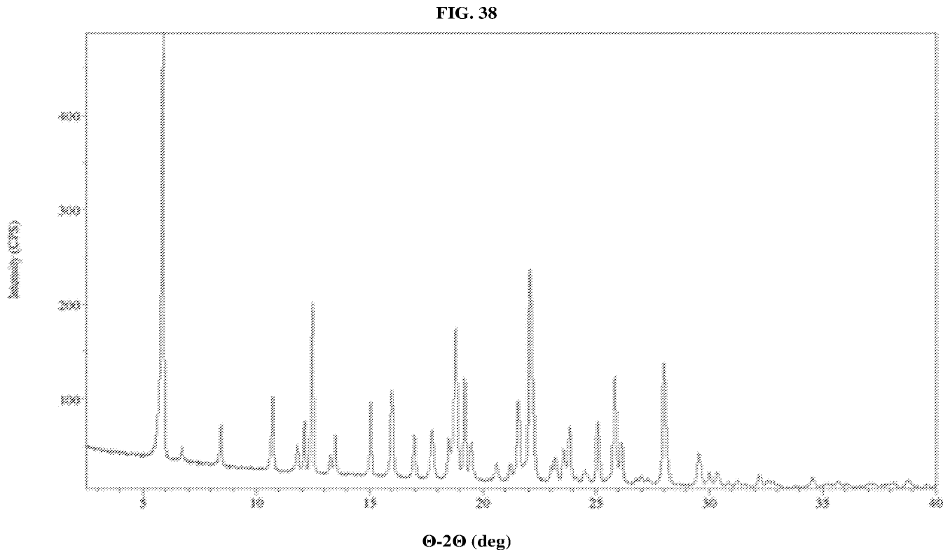

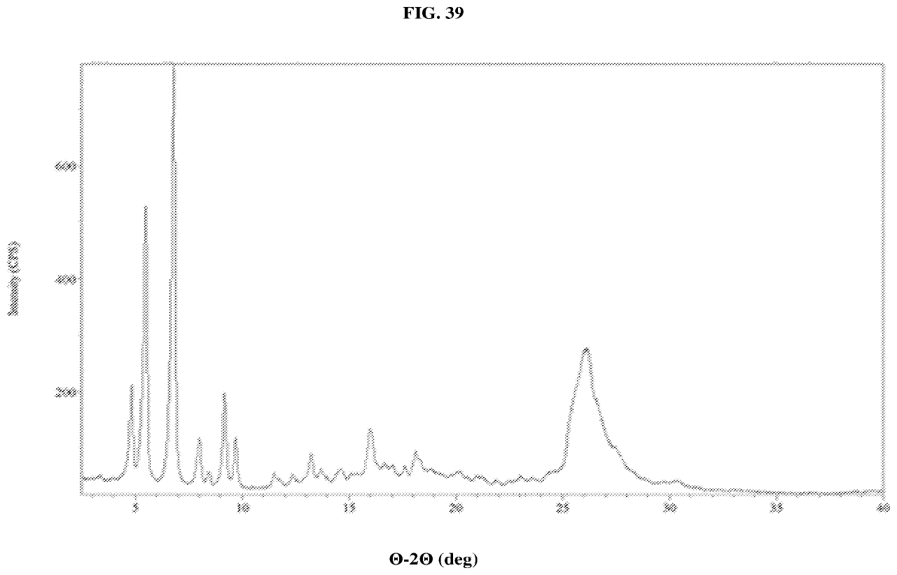

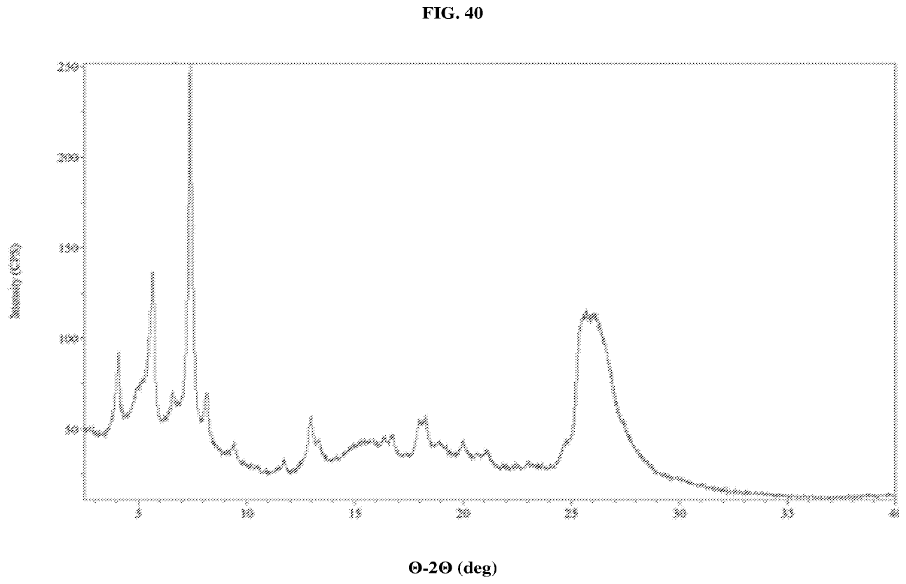

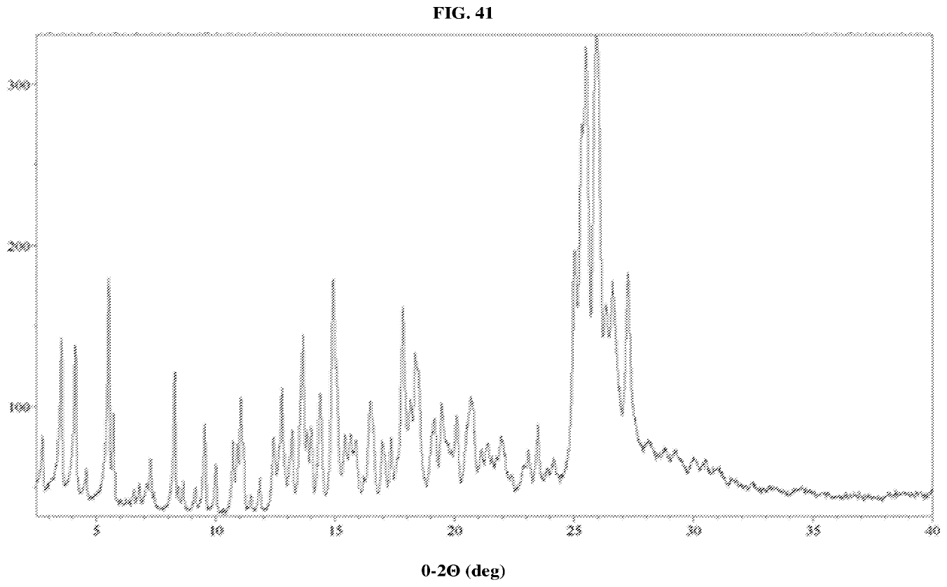

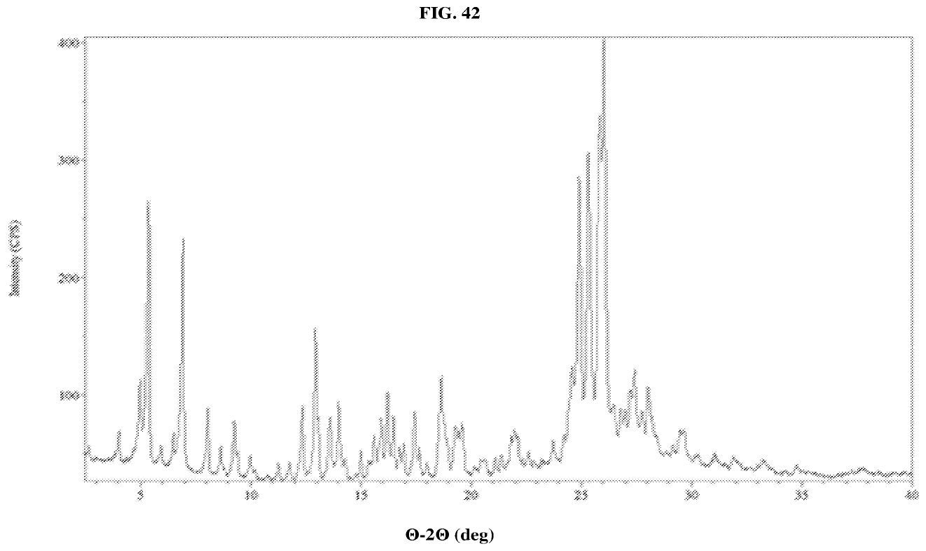

38. The solid form of claim 37, wherein the solid form exhibits peaks or other physical properties as shown in any one of FIG. 1 to FIG. 42.

39. A pharmaceutical composition comprising the solid form of any one of claims 1-38 and a pharmaceutically acceptable carrier, excipient, or adjuvant.
